(19) United States
(12) Patent Application Publication
(10) Pub. No.: US 2026/0185152 A1
(43) Pub. Date: Jul. 2, 2026

(54) Methods For Selective Amplification For Efficient Rearrangement Detection

(71) Applicant: SeQure Dx, Inc., Waltham, MA (US)

(72) Inventors: Douglas SMITH, Waltham, MA (US); Xiaofeng XIN, Waltham, MA (US); Hongyao YU, Waltham, MA (US)

(21) Appl. No.: 19/130,856

(22) PCT Filed: Nov. 17, 2023
(86) PCT No.: PCT/US2023/080328

§ 371 Date: May 16, 2025

Related U.S. Application Data
(60) Provisional application No. 63/384,299, filed on Nov. 18, 2022.

(51) Int. Cl. C12Q 1/6869; C12N 15/10; C12N 15/11; C12Q 1/34; C12Q 1/6806; C12Q 1/6853

(52) U.S. Cl. CPC C12Q 1/6869; C12N 15/1096; C12N 15/11; C12Q 1/34; C12Q 1/6806; C12Q 1/6853; C12N 2310/20; G01N 2333/922

(57) ABSTRACT

The present disclosure relates to development of methods for quantitative detection of genomic rearrangement. In particular, as described in greater detail below, some embodiments of the disclosure provide methods which involve tagmentation in combination with amplification techniques to reduce sample processing times, reduce background, and increase sensitivity for quantitative rearrangement detection.

### CROSS-REFERENCE TO RELATED APPLICATIONS

This application claims the benefit of U.S. Provisional Patent Application No. 63/384,299, filed Nov. 18, 2022, which is incorporated herein by reference in its entirety, including any drawings.

### FIELD

The present disclosure relates to methods for quantitative detection of genomic rearrangements.

### BACKGROUND

For gene editing therapy involving nucleases such as CRISPR/Cas9, cleavage of genomic DNA in live cells results in double strand breaks (DSB), which have the potential of recombining during DSB repair to generate genomic rearrangements. Typically, such rearrangements occur between the target site and frequently edited off-target sites, but they may also occur between two off-target sites, or between the target site or off-target site and a fragile site or any other genomic locus where chromosomal breakage or nicking may occur.

Several molecular methods, including AMP, HTGTS, UDiTaS, CAST-seq, and combinatorial multiplex PCR using rhAmpseq have been applied to enable detection of rearrangements in cells or mammalian tissues following gene editing. However, most of these methods use a low-efficiency DNA shearing and adaptor ligation approach and require significant amounts of genomic DNA as input. One of the methods, UDiTaS, uses a more efficient tagmentation approach, but this method has no enrichment procedures for rearrangements, and thus suffers from low sensitivity for rearrangement detection due to a high background of non-rearranged molecules.

The disclosure provided herein provides methods that employ a combination of tagmentation and negative selection to reduce DNA input requirements, eliminate time-consuming sample processing steps, reduce background resulting from spurious products, and improve sensitivity for quantitative rearrangement detection.

### BRIEF SUMMARY

The present disclosure relates generally to the development of methods for quantitative detection of genomic rearrangements. In particular, as described in greater detail below, some embodiments of the disclosure provide methods which involve tagmentation in combination with amplification techniques to reduce sample processing times, reduce background, and increase sensitivity for quantitative rearrangement detection. The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

Provided herein, among others, includes a method for detecting genome-wide rearrangements in a nucleic acid genome. The method involves (a) contacting a genomic DNA sample obtained from a cell or tissue contacted with a site specific nuclease with a plurality of transposons comprising a first sequence tag at the 5′ end of the transposon, under conditions whereby the plurality of transposons is inserted into the genomic DNA sample and the genomic DNA sample is tagmented into a plurality of nucleic acid fragments each comprising the sequence tag at the 5′ ends of a partially double-stranded nucleic acid fragment. In (b), a first amplification reaction is performed to amplify the re-arranged tagmented nucleic acid fragments using (i) a first target-specific primer comprising a nucleotide sequence complementary to a region adjacent to a target site, (ii) a second primer comprising a nucleotide sequence identical to a portion of the sequence tag located at the 5′ ends of the fragment and (iii) a blocking oligonucleotide comprising a nucleotide sequence complementary to a region on the distal side of the target site relative to the first target-specific primer to produce primary amplification products comprising a nucleotide sequence comprising a re-arranged target sequence. In (c), the primary amplification products are then sequenced.

In some embodiments, the sequence tag comprises a sequence orthogonal to the genome, a unique molecular identifier (UMI), a transposase recognition site, an index sequence, or a combination thereof. In some embodiments, the sequence tag comprises a sequence orthogonal to the genome, a UMI, and a transposase recognition site. In some embodiments, the sequence tag comprises a sequence orthogonal to the genome, a UMI, a transposase recognition site and an index sequence.

In one embodiment, the blocking oligonucleotide comprises an absent or blocked 3′ OH, spacers, inverted nucleotides, or other modifications to block extension of the 3′ end. In one embodiment, the sequence tag comprises uracil.

In one embodiment, the blocking nucleotide comprises one or more phosphothorothioate bonds, spacers, or other modifications at the 3′ and 5′ ends, to block exonuclease digestion at the 3′ and 5′ ends, LNA, BNA, PNA, RNA, DNA, modified nucleic acids, or a combination thereof.

In one embodiment, the first and second primers comprise second and third sequence tags.

In one embodiment, the method disclosed herein further includes prior to (c) performing a second amplification reaction using third and fourth nested primers comprising nucleotide sequences complementary to sequences proximal to the inside ends of the amplified products of (b) and fourth and fifth sequence tags at the 5′ ends of the nested primers to produce secondary amplification products comprising a re-arranged target sequence and additional sequence tags. In one embodiment, the method further includes performing a third amplification reaction using a fifth and sixth primer comprising nucleotide sequences complementary to the amplified products of the second amplification reaction to produce further enriched tertiary amplification products comprising a re-arranged target sequence.

In one embodiment, the third and/or the fourth primers comprise barcode sequences.

In one embodiment, the fifth and sixth primers comprise a sequencing tag and/or an index sequence.

In one embodiment, the method disclosed herein further includes prior to (c) performing a second, hemi-nested amplification reaction using the second primer and a third nested primer wherein the third primer comprises a nucleotide sequence complementary to a sequence proximal to target-specific primer end of the amplified products of (b) and optionally a fourth and fifth sequence tags at the 5′ ends of the second and/or third primers to produce secondary amplification products comprising a re-arranged target sequence and one or two additional sequence tags. In one embodiment, the method further includes performing a third amplification reaction using a fourth and fifth primer comprising nucleotide sequences complementary to the amplified products of the second amplification reaction to produce further enriched tertiary amplification products comprising a re-arranged target sequence.

In one embodiment, the fourth and fifth primers comprise a sequencing tag and/or an index sequence.

In one embodiment, (b) further includes performing a separate amplification reaction to amplify the re-arranged tagmented nucleic acid fragments using a second target specific primer on the opposite side of the target site.

In one embodiment, the method disclosed herein further includes prior to (b) contacting the plurality of tagmented nucleic acid fragments with ddNTP or other 3′ modified dNTP and a DNA polymerase or terminal deoxynucleotidyl transferase to block all extendable 3′ ends.

The present disclosure also includes a method for detecting genome-wide rearrangements in a nucleic acid genome. The method includes (a) contacting a genomic DNA sample obtained from a cell or tissue contacted with a site-specific nuclease with a plurality of transposons comprising a first sequence tag at the 5′ end of the transposon, under conditions whereby the plurality of transposons is inserted into the genomic DNA sample and the genomic DNA sample is tagmented into a plurality of nucleic acid fragments each comprising the sequence tag at the 5′ ends of a partially double-stranded nucleic acid fragment; (b) contacting the plurality of tagmented nucleic acid fragments with a sequence specific cleavage reagent; (c) performing a first amplification reaction to amplify the re-arranged tagmented nucleic acid fragments using (i) a first target-specific primer comprising a nucleotide sequence complementary to a region adjacent to a target site, (ii) a second primer comprising a nucleotide sequence identical to a portion of the sequence tag located at the 5′ ends of the fragment and; (d) sequencing the primary amplification products.

In one embodiment, the sequence specific cleavage reagent is an enzymatic reagent, or a chemical cleavage agent. In one embodiment, the enzymatic cleavage agent comprises a CRISPR Cas/gRNA complex.

In one embodiment, the sequence tag comprises a sequence orthogonal to the genome, a unique molecular identifier (UMI), a transposase recognition site, an index sequence, or a combination thereof. In some embodiments, the sequence tag comprises a sequence orthogonal to the genome, a transposase recognition site, and a UMI. In some embodiments, the sequence tag comprises a sequence orthogonal to the genome, a transposase recognition site, a UMI, and an index sequence.

In one embodiment, the first and second primers comprise second and third sequence tags.

In one embodiment, the method further includes prior to (d) performing a second amplification reaction using third and fourth nested primers comprising nucleotide sequences complementary to sequences proximal to the inside ends of the amplified products of (c) and additional sequence tags at the 5′ ends of the nested primers, to produce secondary amplification products comprising a re-arranged target sequence and additional sequence tags. In one embodiment, the method further includes performing a third amplification reaction using a fifth and sixth primer comprising nucleotide sequences complementary to the amplified products of the second amplification reaction to produce further enriched tertiary amplification products comprising a re-arranged target sequence.

In one embodiment, the third and/or the fourth primers include barcode sequences.

In one embodiment, the fifth and sixth primers include a sequencing tag and/or an index sequence.

In one embodiment, the method further includes prior to (d) performing a second, hemi-nested amplification reaction using the second primer and a third nested primer wherein the third primer comprises a nucleotide sequence complementary to a sequence proximal to target-specific primer end of the amplified products of (c) and optionally a fourth and fifth sequence tags at the 5′ ends of the second and/or third primers to produce secondary amplification products comprising a re-arranged target sequence and one or two additional sequence tags. In one embodiment, the method further includes performing a third amplification reaction using a fourth and fifth primer comprising nucleotide sequences complementary to the amplified products of the second amplification reaction to produce further enriched tertiary amplification products comprising a re-arranged target sequence.

In one embodiment, the second and/or the third primers comprise barcode sequences.

In one embodiment, the fourth and fifth primers comprise a sequencing tag and/or an index sequence.

In one embodiment, the method further includes performing a separate amplification reaction to amplify the re-arranged tagmented nucleic acid fragments using a second target specific primer on the opposite side of the target site.

The present disclosure is also directed to a method for detecting genome-wide rearrangements in a nucleic acid genome. The method includes (a) contacting a genomic DNA sample obtained from a cell or tissue contacted with a site specific nuclease with a plurality of transposons comprising a first sequence tag at the 5′ end of the transposon, wherein the sequence tag comprises an RNA promoter sequence, under conditions whereby the plurality of transposons is inserted into the genomic DNA sample and the genomic DNA sample is tagmented into a plurality of nucleic acid fragments each comprising the sequence tag at the 5′ ends of a partially double-stranded nucleic acid fragment; (b) transcribing the plurality of tagmented nucleic acid fragments into RNA; (c) contacting the RNA with target sequence specific DNA oligonucleotide probes and RNase H; (d) performing a reverse transcription amplification reaction using a (i) a first primer comprising a nucleotide sequence complementary to a portion of the sequence tag located at the 3′ or 5′ end of the fragment and (ii) a second primer comprising a nucleotide sequence complementary to a region adjacent to a target site to produce primary amplification products; (e) sequencing the primary amplification products.

In one embodiment, the sequence tag comprises a sequence orthogonal to the genome, a unique molecular identifier (UMI), a transposase recognition site, an index sequence, or a combination thereof. In some embodiments, the sequence tag comprises a sequence orthogonal to the genome, a transposase recognition site, and a UMI. In some embodiments, the sequence tag comprises a sequence orthogonal to the genome, a transposase recognition site, a UMI, and an index sequence.

In one embodiment, the first and second primers comprise sequence tags.

In one embodiment, the method further includes prior to (e) performing a second amplification reaction using third and fourth nested primers comprising nucleotide sequences complementary to sequences proximal to the inside ends of the amplified products of (d) and additional sequence tags, to produce secondary amplification products comprising a re-arranged target sequence and additional sequence tags. In one embodiment, the method further includes performing a third amplification reaction using a fifth and sixth primer comprising nucleotide sequences complementary to the amplified products of the second amplification reaction to produce further enriched tertiary amplification products comprising a re-arranged target sequence.

In one embodiment, the third and/or fourth primers include barcode sequences.

In one embodiment, the fifth and sixth primers comprise an adapter sequence and/or an index sequence.

In one embodiment, the method further includes prior to (e) performing a second, hemi-nested amplification reaction using the second primer and a third nested primer wherein the third primer comprises a nucleotide sequence complementary to a sequence proximal to target-specific primer end of the amplified products of (d) and optionally a fourth and fifth sequence tags at the 5′ ends of the second and/or third primers to produce secondary amplification products comprising a re-arranged target sequence and one or two additional sequence tags. In one embodiment, the method further includes performing a third amplification reaction using a fourth and fifth primer comprising nucleotide sequences complementary to the amplified products of the second amplification reaction to produce further enriched tertiary amplification products comprising a re-arranged target sequence.

In one embodiment, the second and/or the third primers comprise barcode sequences.

In one embodiment, fourth and fifth primers comprise an adapter sequence and/or an index sequence.

In one embodiment, the method further includes wherein (d) further comprises

performing a separate amplification reaction to amplify the re-arranged tagmented nucleic acid fragments using a second target specific primer on the opposite side of the target site.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows the workflow of an exemplary method of the present disclosure.

FIG. 2 shows the workflow of an exemplary method of the present disclosure.

FIG. 3 shows the workflow of an exemplary method of the present disclosure.

FIG. 4 shows the workflow of an exemplary method of the present disclosure.

FIG. 5 shows the workflow of an exemplary method of the present disclosure.

FIG. 6 shows the workflow of an exemplary method of the present disclosure.

FIG. 7 shows the workflow of an exemplary method of the present disclosure.

FIG. 8 shows the workflow of an exemplary method of the present disclosure.

FIG. 9 shows the workflow of an exemplary method of the present disclosure.

FIG. 10A and FIG. 10B show the blocking effect of blocker oligo.

FIG. 11A and FIG. 11B show the numbers of split reads identified in example 2 of an exemplary method of the present disclosure.

FIG. 12A and FIG. 12B show the validation of an inter-chromosomal translocation identified in the genomic DNA used for all methods of the present disclosure.

FIG. 13A and FIG. 13B show the numbers of split reads identified in example 4 of an exemplary method of the present disclosure.

FIG. 14 shows the numbers of split reads identified in example 6 of an exemplary method of the present disclosure.

FIG. 15A and FIG. 15B show the numbers of split reads identified in example 8 of an exemplary method of the present disclosure.

### DETAILED DESCRIPTION

The present disclosure relates generally to new approaches for detecting genomic rearrangements that are associated with genome editing at specific target sites. These methods address the problem with current detection technologies, due to the requirement of significant amounts of input genomic DNA, low sensitivity, and high background. Provided herein are, inter alia, improved methods for detection of rearranged nucleic acid molecules which involve tagmentation and negative selection techniques that enable high detection sensitivity and cost-efficiency when compared to currently available techniques for detecting genomic rearrangements. For example, some methods of the disclosure involve the steps of tagmentation and subsequent amplification utilizing sequence tags that allow individual molecular recombination events to be identified. Other methods of the disclosure involve the steps of tagmentation, sequence specific cleavage, and subsequent amplification utilizing sequence tags. The present disclosure also provides methods that involve tagmentation, RNA transcription, and subsequent reverse transcription utilizing sequencing tags.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definition

The singular form “a”, “an”, and “the” include plural references unless the context clearly dictates otherwise. “A and/or B” is used herein to include all of the following alternatives: “A”, “B”, “A or B”, and “A and B.”

“Barcode,” as used herein, refers to one or more known nucleotide sequences that are used to identify a nucleic acid with which the barcode is associated. In some embodiments, the barcode sequence enables multiplexing of products derived from different target sites in separate reactions or different samples.

“UMI” (unique molecular identifier) as used herein, refers to one or more randomized or semi-randomized nucleotide sequences that are used to group and deduplicate all products derived from a single starting molecule after sequencing.

“Cleavage”, as used herein, refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or cohesive ends.

As used herein, the term “detecting” a nucleic acid molecule or fragment thereof refers to determining the presence of the nucleic acid molecule, typically when the nucleic acid molecule or fragment thereof has been fully or partially separated from other components of a sample or composition.

As used herein, the term “nuclease” refers to a polypeptide capable of cleaving the phosphodiester bonds between the nucleotide subunits of nucleic acids.

As used herein, the terms “nucleic acid”, “nucleic acid molecule” or “polynucleotide” are used herein interchangeably. They refer to a polymer of deoxyribonucleotides or ribonucleotides in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products. DNAs and RNAs are both polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases (2′-O,4′-C-methylene bridged/locked nucleic acid), biologically modified bases (e.g., methylated bases), intercalated bases, spacers, modified sugars (e.g., 2′-fluororibose, ribose, 2′-deoxyribose, arabinose, and hexose), or modified phosphate groups (e.g., phosphorothioates and 5′-N-phosphoramidite linkages).

As used herein, the term “oligonucleotide” refers to a string of nucleotides or analogues thereof. Oligonucleotides may be obtained by a number of methods including, for example, chemical synthesis, restriction enzyme digestion or PCR. As will be appreciated by one skilled in the art, the length of an oligonucleotide (i.e., the number of nucleotides) can vary widely, often depending on the intended function or use of the oligonucleotide. Generally, oligonucleotides comprise between about 5 and about 300 nucleotides, for example, between about 15 and about 200 nucleotides, between about 15 and about 100 nucleotides, or between about 15 and about 50 nucleotides. Throughout the specification, whenever an oligonucleotide is represented by a sequence of letters (chosen from the four base letters: A, C, G, and T, which denote adenosine, cytidine, guanosine, and thymidine, respectively), the nucleotides are presented in the 5′ to 3′ order from the left to the right. In certain embodiments, the sequence of an oligonucleotide includes one or more degenerate residues described herein.

As used herein, the terms “amplify”, “amplified”, or “amplifying” as used in reference to a nucleic acid or nucleic acid reactions, refers to in vitro methods of making copies of a particular nucleic acid, such as a target nucleic acid, or a tagged nucleic acid produced, for example, by a method described herein.

A “primer” as used herein means a nucleic acid having a sequence complementary and specific to a known sequence in a target or template nucleic acid, e.g., DNA. This means that they must be sufficiently complementary to hybridize with their respective strands to form the desired hybridized products and then be extendable by a DNA polymerase. In some instances, the primer has exact complementarity to the target or template nucleic acid. However, in many situations, exact complementarity is not possible or likely, and one or more mismatches may exist which do not prevent hybridization or the formation of primer extension products using the DNA polymerase.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Although features of the disclosures may be described in the context of a single embodiment, the features may also be provided separately or in any suitable combination. Conversely, although the disclosures may be described herein in the context of separate embodiments for clarity, the disclosures may also be implemented in a single embodiment. Any published patent applications and any other published references, documents, manuscripts, and scientific literature cited herein are incorporated herein by reference for any purpose. In the case of conflict, the present specification, including definitions, will control. In addition, the materials, methods, and examples are illustrative only and not intended to be limiting.

### METHODS OF THE DISCLOSURE

### Method I

As described in greater detail below, one aspect of the present disclosure provides a method for detecting genome-wide re-arrangements in a nucleic acid genome. The method involves (a) contacting a genomic DNA sample obtained from a cell or tissue contacted with a site specific nuclease with a plurality of transposons comprising a first sequence tag at the 5′ end of the transposon, under conditions whereby the plurality of transposons is inserted into the genomic DNA sample and the genomic DNA sample is tagmented into a plurality of nucleic acid fragments each comprising the sequence tag at the 5′ ends of a partially double-stranded nucleic acid fragment. A first amplification reaction is performed to amplify the re-arranged tagmented nucleic acid fragments using (i) a first target-specific primer comprising a nucleotide sequence complementary to a region adjacent to a target site, (ii) a second primer comprising a nucleotide sequence identical to a portion of the sequence tag located at the 5′ ends of the fragment and (iii) a blocking oligonucleotide comprising a nucleotide sequence complementary to a region on the distal side of the target site relative to the first target-specific primer to produce primary amplification products comprising a nucleotide sequence comprising a re-arranged target sequence. The primary amplification products are then sequenced.

### Genomic DNA Samples

The cell or tissue used in the methods provided herein can be any eukaryotic cell type, including but not limited to human cells, non-human primate cells, mammalian cell types, vertebrate cell types, yeast, plant cells. These cells can include, e.g., primary cells and/or tissues, cells or tissues cultured for at least a period of time, or a combination of primary and cultured cells and/or tissues.

In some embodiments, methods described herein is performed on genomic DNA from a single cell. For example, genomic DNA from a single cell can be amplified before performing the methods described herein. Whole genome amplification methods are known in the art. Any of a variety of protocols and/or commercially available kits may be used. Examples of commercially available kits include, but are not limited to, the REPLI-g Single Cell Kit from QIAGEN, GENOMEPLEX® Single Cell Whole Genome Amplification Kit from Sigma Aldrich, Ampli1™ WGA Kit from Silicon Biosystems, and illustra Single Cell GenomiPhi DNA Amplification Kit from GE Healthcare Life Sciences.

Alternatively or additionally, methods disclosed herein can be used with genomic DNA samples from eukaryotic cells and/or tissues or from prokaryotic cells. For example, methods of the present disclosure can be performed using genomic DNA from microorganisms and/or from isolates from patients (e.g., patients receiving antibiotics). In some embodiments, genomic DNA from microbial communities and/or one or more microbiomes is used, e.g., for metagenomic mining. (See, for example, Delmont et al, “Metagenomic mining for microbiologists,” ISME J. 2011 December; 5 (12): 1837-43.)

Genomic DNA can be prepared using any of a variety of suitable methods, including, for example, certain manipulations to cells and/or tissues described herein. Exemplary, non-limiting manipulations include contacting a cell and/or tissue with a nuclease (e.g., a site-specific nuclease and/or an RNA-guided nuclease) or a genome editing system comprising such a nuclease.

### Site Specific Nucleases

As described above, the genomic DNA sample is obtained from a cell or tissue contacted with a site-specific nuclease. Generally, the nuclease is site-specific in that it is known or expected to cleave only at a specific sequence or set of sequences, referred to herein as the nuclease's “target site”.

In methods presently disclosed herein, contacting step(s) with the nuclease are generally carried under out under conditions favorable for the cleavage by the nuclease. That is, even though a given candidate target site or variant target site might not actually be cleaved by the nuclease, the incubation conditions are such that the nuclease would have cleaved at least a significant portion (e.g., at least 1%, at least 10%, at least 20%, at least 30%, at least 40%, at least 50%, at least 60%, at least 70%, at least 80%, at least 90%, or at least 95%) of templates containing its known target site. For known and generally well-characterized nucleases, such conditions are generally known in the art and/or can easily be discovered or optimized. For newly discovered nucleases, such conditions can generally be approximated using information about related nucleases that are better characterized (e.g., homologs and orthologs).

In some embodiments, the nuclease is an endonuclease. In some embodiments, the nuclease is a site-specific endonuclease (e.g., a restriction endonuclease, a meganuclease, a transcription activator-like effector nucleases (TALEN), a zinc finger nuclease, etc.).

In some embodiments, the site specificity of a site-specific nuclease is conferred by an accessory molecule. For example, the CRISPR-associated (Cas) nucleases are guided to specific sites by “guide RNAs” or gRNAs as described herein. In some embodiments, the nuclease is an RNA-guided nuclease. In some embodiments, the nuclease is a CRISPR-associated nuclease.

In some embodiments, the nuclease is a homolog or an ortholog of a previously known nuclease, for example, a newly discovered homolog or ortholog.

In some embodiments, the nuclease is a base editor.

### RNA-Guided Nucleases

RNA-guided nucleases according to the present disclosure include, but are not limited to, naturally-occurring Class 2 CRISPR nucleases such as Cas9, and Cas12a, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cas12a), species (e.g., S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of the protospacer as visualized relative to the guide RNA targeting domain. Cas12a, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cas12a recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389 Sep. 12, 2013 (“Ran”), incorporated by reference herein), or that that do not cut at all.

### Cas9

Crystal structures have been determined for S. pyogenes Cas9 (Jinek et al., Science 343(6176), 1247997, 2014 (“Jinek 2014”), and for S. aureus Cas9 in complex with a unimolecular guide RNA and a target DNA (Nishimasu 2014; Anders et al., Nature. 2014 Sep. 25; 513(7519):569-73 (“Anders 2014”); and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA: DNA recognition, while the REC domain is thought to interact with the repeat:anti-repeat duplex of the gRNA and to mediate the formation of the Cas9/gRNA complex.

The NUC lobe comprises a RuvC domain, an HINH domain, and a PAM-interacting (PI) domain. The RuvC domain shares structural similarity to retroviral integrase superfamily members and cleaves the non-complementary (i.e., bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e., top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

### Cas12a

The crystal structure of Acidaminococcus sp. Cas12a in complex with crRNA and a double-stranded (ds) DNA target including a TTTN PAM sequence has been solved by Yamano et al. (Cell. 2016 May 5; 165 (4): 949-962 (“Yamano”), incorporated by reference herein). Cas12a, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cas12a REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cas 12a share similarities in structure and function, it should be appreciated that certain Cas12a activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cas12a gRNA (the handle) adopts a pseudoknot structure, rather than a stem loop structure formed by the repeat:antirepeat duplex in Cas9 gRNAs.

### Base Editors

Engineered base editors have been developed that provide base-modifying enzyme domains (e.g., deaminases) or reverse transcriptases along with modified CRISPR associated targeting domains, e.g., Cas9 nickase (Gaudelli et al, Nature, 2017, 24644; Komor et al, Nature, 2016,533:420-424: Yang et al, Nat Commun. 2016; 7:13330, Anzalone, Nature. 2019, .doi: 10.1038/s41586-019-1711-4). Such base editors and prime editors typically introduce a single nick on one strand at the target site and then introduce one or more base modifications that are resolved to base edits after DNA replication. It has generally been thought that such enzymes would not induce double strand breaks and consequent rearrangements. However, it has been shown that single-strand nicks can indeed lead to double strand breaks as the result of replisome disassembly and DNA breakage (Vrtis et al, Mol Cell. 2021; 81:1309-1318.e6.) so homologous recombination and large-scale rearrangements may occur because of DNA breakage at sites where DNA nicking occurs. Therefore, the methods of this invention are also applicable to the detection of rearrangements induced by base editors, prime editors, and other site-specific gene editors that introduce single-stranded nicks.

### Nucleic Acids Encoding RNA-Guided Nucleases

Nucleic acids encoding RNA-guided nucleases, e.g., Cas9, Cas12a or functional fragments thereof, are provided herein. Exemplary nucleic acids encoding RNA-guided nucleases have been described previously (see, e.g., Cong et al., Science. 2013 Feb. 15; 339(6121):819-23 (“Cong 2013”); Wang et al., PLoS One. 2013 Dec. 31; 8(12):e85650 (“Wang 2013”); Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in WO 2016/073990 (“Cotta-Ramusino”).

In addition, or alternatively, a nucleic acid encoding an RNA-guided nuclease may comprise a nuclear localization sequence (NLS). Nuclear localization sequences are known in the art.

Guide RNA (gRNA) Molecules

The terms “guide RNA” and “gRNA” refer to any nucleic acid that promotes the specific association (or “targeting”) of an RNA-guided nuclease such as a Cas9 or a Cas 12a to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (“Briner”), which is incorporated by reference), and in Cotta-Ramusino. In bacteria and archea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali 2013”); Jiang et al. Nat Biotechnol. 2013 March; 31(3): 233-239 (“Jiang”); and Jinek et al., 2012 Science August 17; 337(6096):816-821 (“Jinek 2012”), all of which are incorporated by reference herein.)

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”), incorporated by reference herein), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cas12a gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, Feb. 27, 2014 (“Nishimasu 2014”) and Nishimasu et al., Cell 162, 1113-1126 Aug. 27, 2015 (“Nishimasu 2015”), both incorporated by reference herein). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or may in the future be) discovered or invented which utilize gRNAs that differ in some ways from those described to this point. For instance, Cas12a (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (“Zetsche I”), incorporated by reference herein). A gRNA for use in a Cas12a genome editing system generally includes a targeting domain and a complementarity domain (alternately referred to as a “handle”). It should also be noted that, in gRNAs for use with Cas12a, the targeting domain is usually present at or near the 3′ end, rather than the 5′ end as described above in connection with Cas9 gRNAs (the handle is at or near the 5′ end of a Cas 12a gRNA).

Those of skill in the art will appreciate, however, that although structural differences may exist between gRNAs from different prokaryotic species, or between Cas12a and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cas12a. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

### Transposons and Tagmentation

In the first step, a genomic DNA sample obtained from a cell or tissue contacted with a site-specific nuclease is contacted with a plurality of transposons to introduce known DNA sequences onto the ends of genomic DNA fragments. For example, Tn5 transposase catalyzes strand transfer via nucleophilic attack on the target DNA by activated 3-OH groups at the transposon ends, leaving a 9-bp gap at the target site (Vaezeslami et al, J Bacteriol, 2007; 189:7436-7441). A transposome is a complex of a transposase enzyme and DNA which comprises a transposon end sequence (also known as “transposase recognition sequence” or “mosaic end” (ME)). When a double-stranded oligonucleotides with a transposon recognition sequence at one end are provided, transposition results in the introduction of free DNA ends into the target molecule (e.g., “tagmentation”; Adey et al, Genome Biol. 2010; 11: R119). This system can be adapted using hyperactive transposase enzymes and modified DNA oligonucleotides (sequence tags) comprising MEs to introduce tags on both strands of the tagmented products with functional DNA molecules (e.g., primer binding sites). Any transposase enzyme with tagmentation activity, e.g., any transposase enzyme capable of mediating strand transfer and integration of oligonucleotides (e.g., tags) at the ends of the tagmented DNA, can be used. In some embodiments, the transposase is any transposase capable of conservative transposition. In some embodiments, the transposase is a cut and paste transposase. Other kinds of transposase are known in the art and are within the scope of this disclosure. For example, suitable transposase enzymes include, without limitation, Tn5, Tn5059, Mos-1, HyperMu™, Hermes, Tn7, or any functional variant or derivative of the previously listed transposase enzymes.

For instance, the Tn5 transposase may be produced as purified protein monomers. Tn5 transposase is also commercially available (e.g., manufacturer Illumina, Illumina.com, Catalog No. 15027865, TD Tagment DNA Buffer Catalog No. 15027866). These can be subsequently loaded with the oligonucleotides of interest, e.g., ssDNA oligonucleotides containing MEs for Tn5 recognition and additional functional sequences (e.g., primer binding sites, e.g., UMIs) are annealed to form a dsDNA mosaic end oligonucleotide (MEDS) that is recognized by Tn5 during dimer assembly (e.g., transposome dimerization). In some embodiments, a hyperactive Tn5 transposase can be loaded with tags (e.g., oligonucleotides of interest) which can simultaneously fragment and tag a genome with the desired sequences.

As described above, the transposons include a first sequence tag at the 5′ end of the transposon. As used herein, the first sequence tag refers to non-target nucleic acid component, generally DNA, that provides a means of addressing a nucleic acid fragment to which it is joined. For example, in some embodiments, a sequence tag is or comprises a nucleotide sequence that permits identification, recognition, and/or molecular or biochemical manipulation of the DNA to which the tag is attached (e.g., by providing a site for annealing an oligonucleotide, such as a primer for extension by a DNA polymerase, or an oligonucleotide for capture or for a ligation reaction). In some embodiments, sequencing tags are used for generation of templates for next-generation sequencing for a particular sequencing platform (e.g., sequencing tags for: an Illumina sequencing platform; for a Thermo Fisher Ion Torrent sequencing platform; for a Pacific Biosciences' Sequel sequencing platform; for a MGI sequencing platform; or for any other sequencing platform). In some embodiments, a sequencing tag is a full-length Illumina forward (15) adapter. In some embodiments, a sequencing tag is a full-length Illumina reverse (17) adapter. In some embodiments, the sequence tag includes a unique sequence (e.g. a sequence orthogonal to the genome), a unique molecular identifier (UMI), a transposase recognition site, an index sequence, or a combination thereof. In some embodiments, the sequence tag includes a sequence orthogonal to the genome, a unique molecular identifier (UMI), and a transposase recognition site. In some embodiments, the sequence tag includes a sequence orthogonal to the genome, a unique molecular identifier (UMI), an index sequence, and a transposase recognition site.

In some embodiments, the UMI is a randomly generated sequence. In some embodiments, the UMI is between eight and 20 nucleotides in length, for example, between 10 and 16 nucleotides in length, such as 10, 11, 12, 13, 14, 15, and 16 nucleotides in length. The production and use of UMIs in various contexts are known in the art.

Tagmentation is a common initial step for all methods described herein. In some embodiments, the step of fragmenting the genomic DNA in cells of the biological sample comprises contacting the biological sample containing the genomic DNA with the transposase enzyme (e.g., a transposome, e.g., a reaction mixture (e.g., solution)) including a transposase), under any suitable conditions. In some embodiments, the transposome is assembled by annealing transposon-end containing oligonucleotides for about 1, 2, 3, 4, 5, 6, 7, 8, 9, or 10 minutes at about 95, 96, 97, 98, or 99° C., followed by about 40, 50, 60, 70, 80, 90, 100, 200, 300, 400, or 500 cycles of 1 minutes at temperatures of about 80, 85, 90, 95, or 99° C. while decreasing temperature 0.2, 0.3, 0.4, 0.5, 0.6, 0.7, 0.8, 0.9, 1, or 2° C. every cycle. The transposase and annealed oligonucleotides can then be incubated at 15, 20, 25, 30, 35, 40° C. for 5, 10, 15, 20, 25, 30, 35, 40, 45, 50, 55, 60, 65, 70, 75, 80, 85, 90, 95, 100, 105, 110, 115, 120 minutes. In some embodiments, such suitable conditions result in the fragmentation (e.g., tagmentation) of the genomic DNA of cells present in the biological sample. Typical conditions will depend on the transposase enzyme used and can be determined using routine methods known in the art. Therefore, suitable conditions can be conditions (e.g., buffer, salt, concentration, pH, temperature, time conditions) under which the transposase enzyme is functional, e.g., in which the transposase enzyme displays transposase activity, particularly tagmentation activity, in the biological sample. In some embodiments, the tagmentation reaction involves mixing about 5, 10, 15, 20, 25, 30, 35, 40, 45, 50, 55, 60, 65, 70, 75, 80, 85, 90, 95, 100, 200, 300, 400, or 500 ng genomic DNA with about 0.1, 0.2, 0.3, 0.4, 0.5, 0.6, 0.7, 0.8, 1, 2, 3, 4, 5, 6, 7 or 8 μL of the transposome and incubation at 30, 35, 40, 45, 50, 55, 60, or 65° C. for about 5, 10, 15, 20, 23, 30, 35, 40, 45, 50, 55, 60, 65, 70, 75, 80, 85, 90, 95, 100, 105, 110, 115, or 120 minutes.

In one non-limiting example, the reaction mixture comprises a transposase enzyme in a buffered solution (e.g., Tris-acetate) having a pH of about 6.5 to about 8.5, e.g., about 7.0 to about 8.0 such as about 7.5. Additionally or alternatively, the reaction mixture can be used at any suitable temperature based on the temperature optimum of the transposase enzyme, such as about 10° to about 55° C., e.g., about 10° to about 54°, about 11° to about 53°, about 12° to about 52°, about 13° to about 51°, about 14° to about 50°, about 15° to about 49°, about 16° to about 48°, about 17° to about 47° C., e.g., about 10°, about 12°, about 15°, about 18°, about 20°, about 22°, about 25°, about 28°, about 30°, about 33°, about 35°, about or 37° C., preferably about 30° to about 40° C., e.g., about 37° C. for Tn5. In some embodiments, the transposase enzyme can be contacted with the biological sample for about 10 minutes to about one hour. In some embodiments, the transposase enzyme can be contacted with the biological sample for about 20, about 30, about 40, or about 50 minutes. In some embodiments, the transposase enzyme can be contacted with the biological sample for about 1 hour to about 4 hours.

Briefly, in some embodiments, Tn5 tagmentation uses staggered transposon ends with an optional 3′ extension block (e.g., a 3′ dideoxy nucleotide, inverted nucleotide, or amino modifier) and 5′ sequence tags. As described above, in some embodiments, the sequence tag harbors a UMI that typically comprises a random 12-nucleotide sequences that differs between each transposon end. The UMIs allow the products generated from a single starting genomic fragment to be identified, grouped and analyzed together after multiple amplification steps. This allows individual molecular recombination events to be identified, and their frequency of occurrence in the population to be quantified relative to other unique events. It also allows variants associated with each haplotype in the starting genome to be identified. The use of tagmentation also avoids inefficient and time-consuming DNA shearing, end repair and adaptor ligation steps, reducing input DNA requirements, and speeding up the process.

In the method described herein, once the plurality of transposons is inserted into the genomic DNA sample, the genomic DNA sample is tagmented into a plurality of nucleic acid fragments each comprising the sequence tag at the 5′ ends of a partially double-stranded nucleic acid fragment.

### Amplification

In the method described herein, after tagmentation, a first amplification reaction is performed to amplify the re-arranged tagmented nucleic acid fragments using (i) a first target-specific primer comprising a nucleotide sequence complementary to a region adjacent to a target site, (ii) a second primer comprising a nucleotide sequence identical to a portion of the sequence tag located at the 5′ ends of the fragment and (iii) a blocking oligonucleotide comprising a nucleotide sequence complementary to a region on the distal side of the target site relative to the first target-specific primer to produce primary amplification products comprising a nucleotide sequence comprising a re-arranged target sequence.

Numerous methods of amplifying nucleic acids are known in the art, and amplification reactions include polymerase chain reactions (PCR), ligase chain reactions, strand displacement amplification reactions, rolling circle amplification reactions, transcription-mediated amplification methods such as NASBA (e.g., U.S. Pat. No. 5,409,818), loop mediated amplification methods (e.g., “LAMP” amplification using loop-forming sequences, e.g., as described in U.S. Pat. No. 6,410,278). A nucleic acid that is amplified can be DNA comprising, consisting of, or derived from DNA or RNA or a mixture of DNA and RNA, including modified DNA and/or RNA. The products resulting from amplification of a nucleic acid molecule or molecules (i.e., “amplification products”), whether the starting nucleic acid is DNA, RNA or both, can be either DNA or RNA, or a mixture of both DNA and RNA nucleosides or nucleotides, or they can comprise modified DNA or RNA nucleosides or nucleotides. A “copy” does not necessarily mean perfect sequence complementarity or identity to the target sequence. For example, copies can include nucleotide analogs such as deoxyinosine or deoxyuridine, intentional sequence alterations (such as sequence alterations introduced through a primer comprising a sequence that is hybridizable, but not complementary, to the target sequence), and/or sequence errors that occur during amplification. In some embodiments, the first amplification reaction is a PCR.

Protocols for amplification can include, e.g., one round of amplification or multiple rounds of amplification. For example, a first amplification round can be followed by a second amplification round with or without one or more processing (e.g., cleanup, concentrating, etc.) steps in between the two rounds of amplification. Additional rounds of amplification may be used in some embodiments, with or without one or more processing steps in between.

The number of amplification cycles can be varied depending on the embodiment. For example, each round of amplification can comprise at least 4, 8, 10, 12, 14, 16, 18, 20, 22, 24, 26, 28, 30, 32, 34, 38, or 40 cycles. In embodiments involving more than one round of amplification, the number of amplification cycles used in each round can be the same or it can be different. In cases in which two rounds of amplification are used and the number of amplification cycles in the two rounds differ, the first round can comprise more cycles or it may comprise fewer cycles than the second round. As an example, a first round of amplification can comprise 12 cycles and a second round of amplification can comprise 15 cycles. As another example, a first round of amplification can comprise 10 cycles and a second round of amplification can comprise 12 cycles.

The temperature of the amplification reaction will vary depending upon the step of the reaction. In some embodiments, an initial denaturing can be performed at about 90, 91, 92, 93, 94, 95, 96, 97, 98, or 99° C. for about 1, 2, 3, 4, or 5 minutes, and a final extension can be performed at about 67, 68, 69, 70, 71, 72, 73, 74, or 75° C. for about 1, 2, 3, 4, 5, 6, 7, 8, 9, or 10 minutes.

### Blocking Oligonucleotides

As described above, the first amplification reaction is performed to amplify the re-arranged tagmented nucleic acid fragments using (i) a first target-specific primer comprising a nucleotide sequence complementary to a region adjacent to a target site, (ii) a second primer comprising a nucleotide sequence identical to a portion of the sequence tag located at the 5′ ends of the fragment and (iii) a blocking oligonucleotide comprising a nucleotide sequence complementary to a region on the distal side of the target site relative to the first target-specific primer.

As used herein, a blocking oligonucleotide is an engineered single stranded nucleic acid sequence. The blocking oligonucleotide can be comprised of single stranded DNA, RNA, peptide nucleic acid (PNA), locked nucleic acid (LNA), bridged-nucleic acid (BNA), and/or other modified nucleotides. In some embodiments, it is a DNA oligonucleotide that includes multiple modified bases. The blocking oligonucleotide may comprise 10 to 100 nucleotides, but is preferably between 15 and 50 nucleotides in length.

The blocking oligonucleotide has strong avidity for the template DNA but cannot be extended by the polymerase used for amplification due to an absent or blocked 3′OH, and modifications at or near the 3′ end that confer resistance to any 3′>5′ exonuclease activity of the polymerase (e.g., multiple phosphorothioate bonds at the 3′ end, spacers, inverted nucleotides, LNA, or BNA residues). The blocking oligonucleotide must be complementary to the same strand as the first target specific primer and must hybridize on the distal side of the gene editor target site relative to the location of the first target specific primer. In one embodiment, the blocking oligonucleotide includes an absent or blocked 3′ OH, spacers, inverted nucleotides, or other modifications to block extension of the 3′ end.

In one embodiment, the blocking oligonucleotide comprises one or more phosphothorothioate bonds, spacers, or other modifications at the 3′ and 5′ ends, to block polymerase extension and exonuclease digestion, and a backbone of DNA, RNA, LNA, BNA, PNA, modified nucleic acids, or a combination thereof.

In some embodiments, the blocking nucleotide includes nucleotides that increase duplex stability and melting temperature relative to native DNA. This enables hybridization of the blocking oligonucleotide at a higher temperature than the first and second primers (and therefore enabling hybridization to the template before the primers are able to hybridize during amplification). High duplex stability also reduces the likelihood of dissociation of the blocking oligonucleotide and read-through by the polymerase during primer extension.

In some embodiments, the blocking oligonucleotide includes a chemical- or photo-crosslinking moiety to permit efficient covalent attachment to the hybridized DNA strand, preventing polymerase extension and dissociation. Exemplary chemical or photo-crosslinking moieties include, without limitation, psoralen, click chemistry, and 3-cyanovinylcarbazole.

### Sequencing

The use of the blocking oligonucleotide, first target-specific primer, and second primer in the amplification reaction produces primary amplification products comprising a nucleotide sequence comprising a re-arranged target sequence. The primary amplification products are then sequenced.

As used herein, “sequencing” includes any method of determining the sequence of a nucleic acid. Any method of sequencing can be used in the present methods, including chain terminator (Sanger) sequencing and dye terminator sequencing. In preferred embodiments, Next Generation Sequencing (NGS), a high-throughput sequencing technology that performs thousands or millions of sequencing reactions in parallel, is used. Although different NGS platforms use varying assay chemistries, they all generate sequence data from a large number of sequencing reactions run simultaneously on a large number of templates. Typically, the sequence data is collected using a scanner, and then assembled and analyzed bioinformatically. Thus, the sequencing reactions are performed, read, assembled, and analyzed in parallel. Exemplary approaches, systems, or techniques may be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification. Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, Ion Torrent semiconductor sequencing, SMRT® sequencing, and Oxford Nanopore Technology sequencing.

In some embodiments, the primary reaction products can be purified to remove primers, and quantified. In some embodiments, the sample can be split, and a symmetrical set of primers would be used to evaluate both sides of the target site for rearrangements in separate reactions (for this and subsequent steps). The primary reaction products can also be used to detect rearrangements at other genomic loci, such as candidate or bona fide off-target sites predicted by in silico algorithms, or detected by other experimental methods.

### Additional Amplification

As described above, the first amplification round can be followed by a second amplification round. Thus, in some embodiments, the second amplification includes nested PCR and the introduction of second and third sequence tags. In some embodiments, the second amplification includes hemi-nested PCR.

In accordance with such embodiments where a second amplification round is used, the method disclosed herein can further include prior to step (c) performing a second amplification reaction using third and fourth nested primers. The third and fourth primers include nucleotide sequences complementary to sequences proximal to the inside ends of the amplified products of (b) and fourth and fifth sequence tags at the 5′ ends of the nested primers. The second amplification reaction produces secondary amplification products that include a re-arranged target sequence and additional sequence tags. This nested amplification step enriches for the desired products, since the truncated extension products, and spurious fragments derived from mis-priming in the first amplification step, cannot serve as amplification templates.

In some embodiments where a second amplification round is used, the method disclosed herein can further include prior to (c) performing a second, hemi-nested amplification reaction using the second primer and a third nested primer wherein the third primer comprises a nucleotide sequence complementary to a sequence proximal to target-specific primer end of the amplified products of (b) and optionally a fourth and fifth sequence tags at the 5′ ends of the second and/or third primers to produce secondary amplification products comprising a re-arranged target sequence and one or two additional sequence tags.

For the second amplification step, protocols for amplification can include, e.g., one round of amplification or multiple rounds of amplification. For example, a first amplification round can be followed by a second amplification round with or without one or more processing (e.g., cleanup, concentrating, etc.) steps in between the two rounds of amplification. Additional rounds of amplification may be used in some embodiments, with or without one or more processing steps in between.

The number of amplification cycles can be varied depending on the embodiment. For example, each round of amplification can comprise at least 4, 8, 10, 12, 14, 16, 18, 20, 22, 24, 26, 28, 30, 32, 34, 38, or 40 cycles. In embodiments involving more than one round of amplification, the number of amplification cycles used in each round can be the same or it can be different. In cases in which two rounds of amplification are used and the number of amplification cycles in the two rounds differ, the first round can comprise more cycles or it may comprise fewer cycles than the second round. As an example, a first round of amplification can comprise 12 cycles and a second round of amplification can comprise 15 cycles. As another example, a first round of amplification can comprise 10 cycles and a second round of amplification can comprise 12 cycles.

The temperature of the amplification reaction will vary depending upon the step of the reaction. In some embodiments, an initial denaturing can be performed at about 90, 91, 92, 93, 94, 95, 96, 97, 98, or 99° C. for about 1, 2, 3, 4, or 5 minutes, and a final extension can be performed at about 67, 68, 69, 70, 71, 72, 73, 74, or 75° C. for about 1, 2, 3, 4, 5, 6, 7, 8, 9, or 10 minutes.

In one embodiment, the method further includes performing a third amplification reaction using a fifth and sixth primer, or a fourth and fifth primer after hemi-nested PCR, comprising nucleotide sequences complementary to the amplified products of the second amplification reaction to produce further enriched tertiary amplification products comprising a re-arranged target sequence. In some embodiments, the third amplification reaction is a hemi-nested PCR.

For the third amplification step, protocols for amplification can include, e.g., one round of amplification or multiple rounds of amplification. For example, a first amplification round can be followed by a second amplification round with or without one or more processing (e.g., cleanup, concentrating, etc.) steps in between the two rounds of amplification. Additional rounds of amplification may be used in some embodiments, with or without one or more processing steps in between.

The number of amplification cycles can be varied depending on the embodiment. For example, each round of amplification can comprise at least 4, 8, 10, 12, 14, 16, 18, 20, 22, 24, 26, 28, 30, 32, 34, 38, or 40 cycles. In embodiments involving more than one round of amplification, the number of amplification cycles used in each round can be the same or it can be different. In cases in which two rounds of amplification are used and the number of amplification cycles in the two rounds differ, the first round can comprise more cycles or it may comprise fewer cycles than the second round. As an example, a first round of amplification can comprise 12 cycles and a second round of amplification can comprise 15 cycles. As another example, a first round of amplification can comprise 10 cycles and a second round of amplification can comprise 12 cycles.

The temperature of the amplification reaction will vary depending upon the step of the reaction. In some embodiments, an initial denaturing can be performed at about 90, 91, 92, 93, 94, 95, 96, 97, 98, or 99° C. for about 1, 2, 3, 4, or 5 minutes, and a final extension can be performed at about 67, 68, 69, 70, 71, 72, 73, 74, or 75° C. for about 1, 2, 3, 4, 5, 6, 7, 8, 9, or 10 minutes.

In one embodiment, the third and/or the fourth primers comprise barcode sequences.

### Barcode Sequences

A “barcode” is a molecular label, or identifier, that conveys or is capable of conveying information about a sample, sequencing read, or group of samples or sequencing reads (e.g., information about an analyte in a sample, a template, a bead, a microwell, a primer, a read, and/or a capture probe). A barcode can be part of an analyte, or independent of an analyte. A barcode can be attached to an analyte. A particular barcode will be unique relative to other barcodes, and may include error-correcting features (e.g., Hamming codes) that enable correct identification of each barcode in a mixture even in the presence of sequencing errors.

As used in here Barcodes comprise custom polynucleotides of defined sequence that are introduced by primer extension (during PCR, for example) or by adapter ligation. Barcodes can allow for identification and/or quantification of groups of sequencing reads that share the same barcode. Barcodes can be 8 to 50 or more bases in length, but are typically between 10 and 25 bases in length.

Barcodes can be used to spatially-resolve molecular components found in biological samples, for example, at single-cell resolution (e.g., a barcode can be or can include a “spatial barcode”). In some embodiments, a barcode includes two or more sub-barcodes that can together function as a single barcode. For example, a polynucleotide barcode can include two or more polynucleotide sequences (e.g., sub-barcodes) that are separated by one or more non-barcode sequences.

An index sequence is a class of barcode that is used to differentiate and group sets of reads derived from different samples after sequencing, by means of standard sequencing analysis software.

### Unique Molecular Identifiers

A unique molecular identifier, or “UMI”, comprises a short stretch of random nucleotides (e.g., NNNNNNNN (SEQ ID NO: 90)) or semi-random nucleotides (e.g., NWYYRRV (SEQ ID NO: 91)) included in a custom primer or adapter. UMIs are typically between 6 and 25 bases, and are generally introduced by tagmentation or adapter ligation, although they may also be introduced by a single round of primer extension. UMIs serve to enable the identification and grouping of extension or amplification products (e.g., PCR products) that derive from a single starting genomic DNA molecule. Such grouping enables counting of unique molecules in a starting sample that are represented by multiple reads after amplification and sequencing (e.g., counting of unique recombination events). UMIs can also be used to distinguishing between SNVs and sequencing errors in amplified molecules.

Individual sequencing reads may contain multiple barcodes, indexes and UMIs in addition to the sequences being characterized.

In one embodiment, the fifth and sixth primers comprise a sequencing tag and/or an index sequence.

In one embodiment, (b) further includes performing a separate amplification reaction to amplify the re-arranged tagmented nucleic acid fragments using a second target specific primer on the opposite side of the target site.

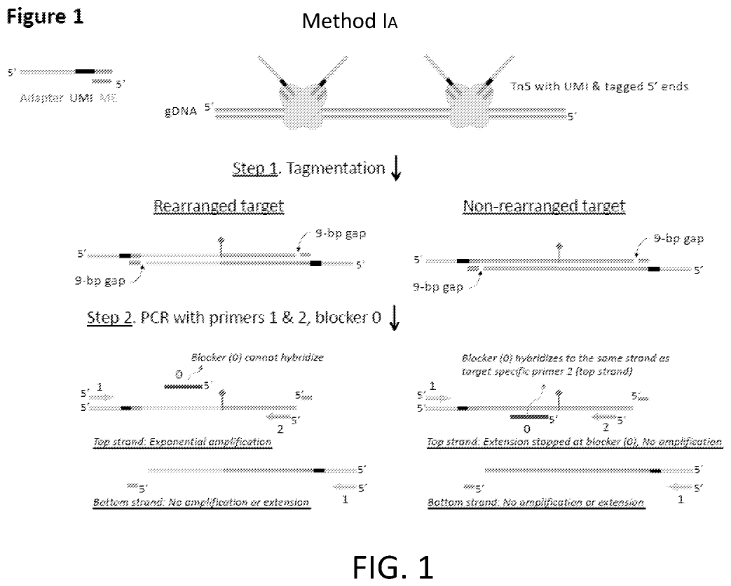

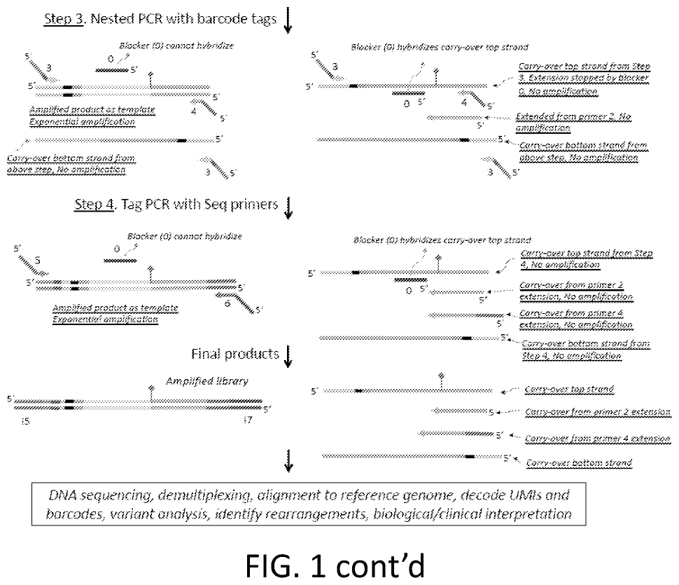

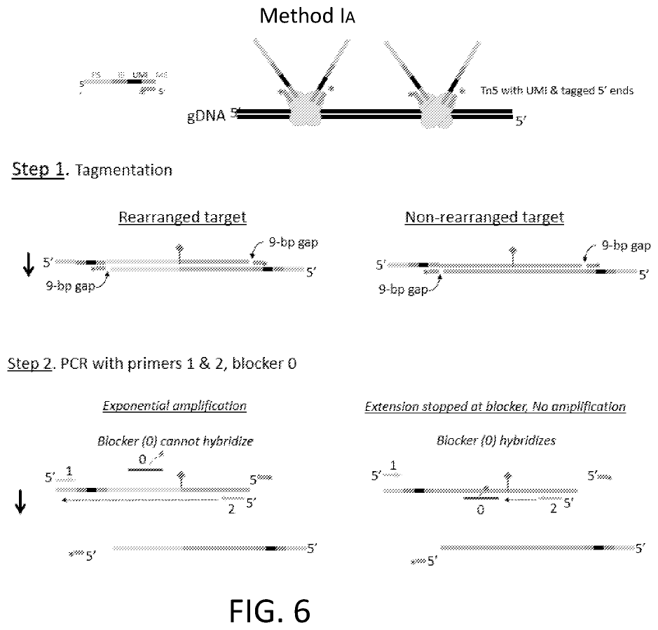

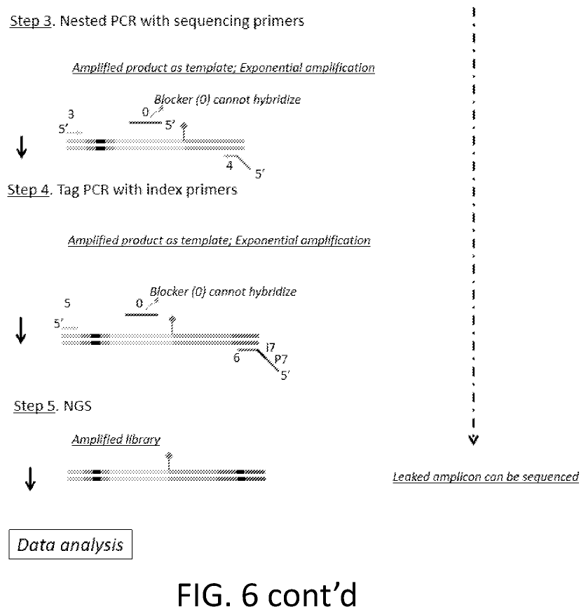

Embodiments of the method described herein is shown in FIG. 1 and FIG. 6 (Method IA). In some embodiments, the sequence tag comprises a sequence orthogonal to the genome, a UMI, and transposase recognition site (FIG. 1). In some embodiments, the sequence tag comprises a sequence orthogonal to the genome, a UMI, an index sequence, and transposase recognition site (FIG. 6). As shown in FIG. 1 and FIG. 6, after tagmentation, the sample can be subjected to a three-step PCR amplification procedure to enrich for fragments that have undergone recombination (Steps 2-4). Each of these steps is carried out in the presence of a blocking oligonucleotide, which prevents the products from native (non-rearranged) sequences from undergoing exponential amplification. The amplification procedure may include a 3-step or touchdown PCR cycling protocol to ensure that the blocking oligonucleotide hybridizes before the PCR primers are extended.

In the first PCR step (Step 2), one DNA primer (labeled as “1” in FIG. 1 and FIG. 6) matches the 5′ transposon end tag (e.g., sequencing tag) and the other (labeled as “2” in FIG. 1 and FIG. 6) is complementary to a region flanking the gene editor target site that is being investigated as a possible site of rearrangements. In the presence of the blocker (labeled as “0”), DNA polymerase extension products derived from the target specific primer (2) at a native gene editor target site (right hand side of FIG. 1 and FIG. 6, below) are blocked from extension. This prevents exponential PCR amplification from occurring (note that the 3′ ends of the tagmented fragments cannot be extended if we include the optional 3′ end blocking step). If a rearrangement has occurred such that the sequence complementary to the blocking oligonucleotide has been replaced by another sequence derived from elsewhere in the genome, the blocker cannot hybridize, and PCR products derived from the rearranged fragment are productively generated (left hand side of FIG. 1 and FIG. 6). Following amplification, the PCR products are purified to remove primers, and are quantified. Typically, the sample would be split, and a symmetrical set of primers would be used to evaluate both sides of the target site for rearrangements in separate reactions (for this and subsequent steps). The PCR products can be further aliquoted and used to detect rearrangements at other genomic loci, such as the off-target sites predicted by in silico algorithms, or detected by other experimental methods.

In the second PCR step (Step 3), a pair of nested primers (labeled as “3” and “4”, respectively in FIG. 1) are used that are complementary to sequences proximal to the inside ends of the PCR product generated in the Step 2. This nested PCR step enriches for the desired products, since the truncated extension products, and spurious fragments derived from mis-priming at other loci in the first PCR step, cannot serve as amplification templates. The nested primers contain sequence tags that can be used as priming sites for the third PCR step (Step 4), and may also contain barcodes to enable multiplexing of products derived from different target sites in separate reactions or different samples. Alternatively, in cases where the first sequencing tag includes an index primer (FIG. 6), the second PCR step involves a hemi-nested PCR step using a pair of primers (labeled as “3” and “4” in FIG. 6) in which primer “4” is complementary to a sequence proximal to the inside end of the PCR product generated in the Step 2, and primer “3” matches the 5′ transposon end tag (e.g., sequencing tag). Once again, in the presence of the blocking oligonucleotide (blocker 0), only the PCR products derived from rearranged target molecules can be amplified further with the nested or hemi-nested primers. Fragments derived from native, non-rearranged target sites cannot be amplified (only a single, truncated extension product can be generated in each PCR cycle). Following amplification, the PCR products are purified to remove primers and quantified.

In the third PCR step (Step 4), primers (labeled “5” and “6”) complementary to the tags that were introduced by PCR in the second PCR step (Step 3) are employed to enrich further for rearranged molecules. These primers also provide the sequences required for NGS sequencing, such as the adapter sequences for cluster generation on an Illumina flow cell, UMIs and index sequences for sample multiplexing. Following amplification, the PCR products are purified to remove primers and quantified. The resulting library is then subjected to NGS sequencing on the Illumina platform, for example.

### Variations of Method I

In one embodiment, the method disclosed herein further includes prior to (b) contacting the plurality of tagmented nucleic acid fragments with ddNTP or other 3′ modified dNTP and a DNA polymerase (e.g., Klenow fragment or terminal deoxynucleotidyl transferase) to block all extendable 3′ ends immediately after tagmentation.

Tagementation and amplification reactions can be performed as described above for Method I.

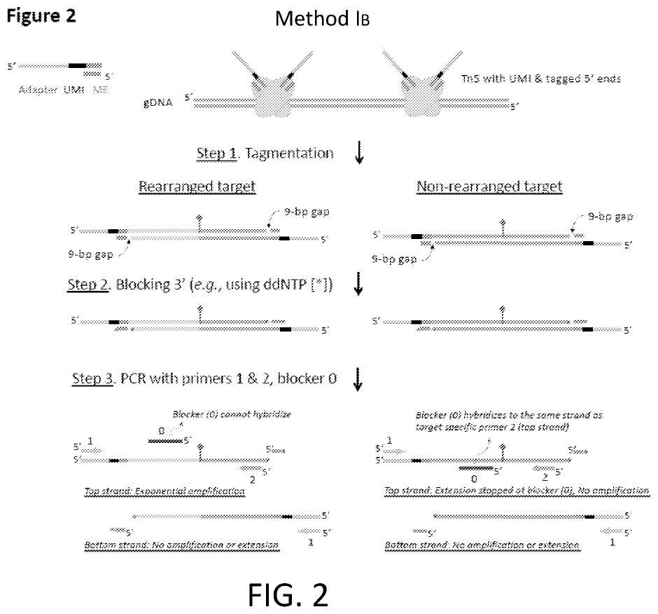

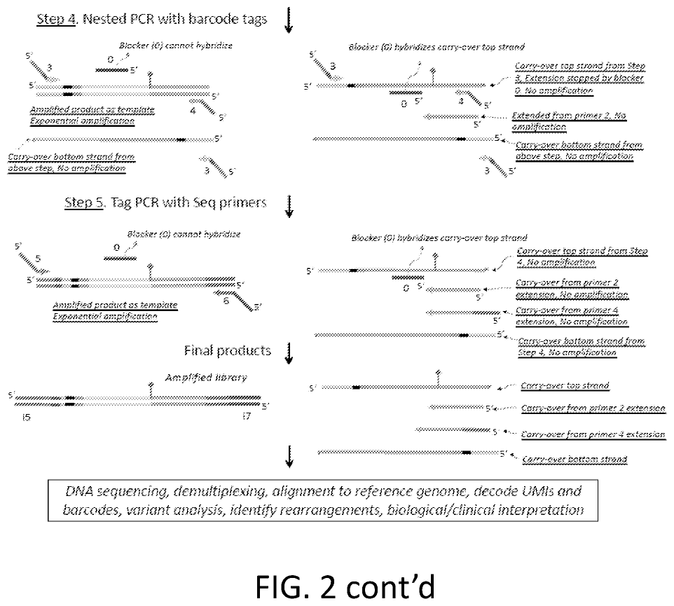

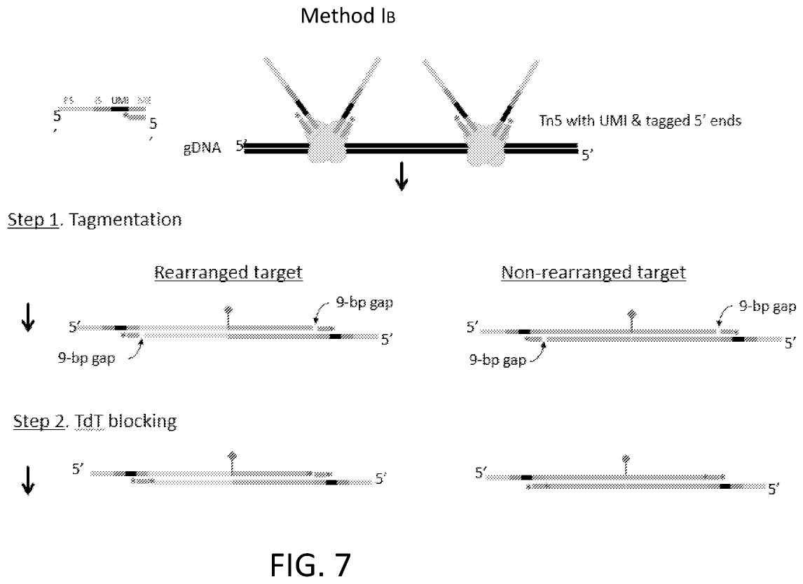

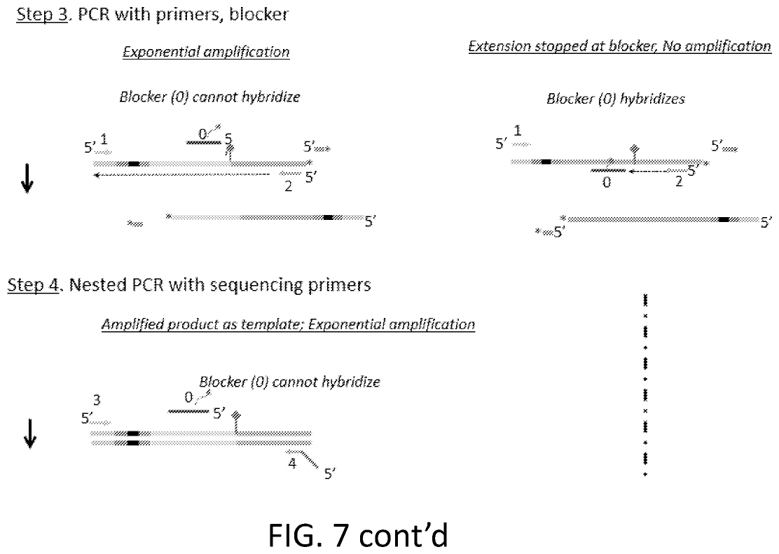

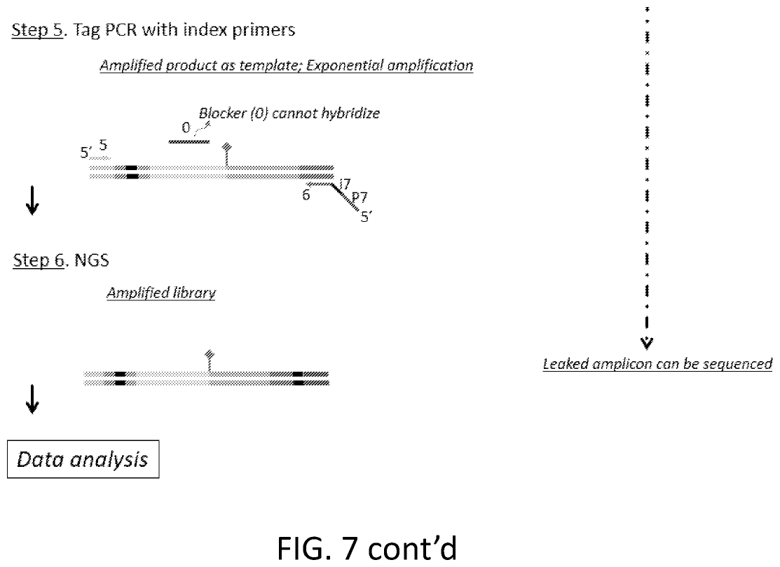

An exemplary embodiment of the method described herein is shown in FIG. 2 (Method I_{B}). As shown in FIG. 2 and FIG. 7, after tagmentation, 3′ end of the short ME fragment and the 3′ end of genomic DNA at the 9-bp gap are extended and blocked by ddNTP with DNA polymerase or terminal deoxynucleotidyl transferase (TdT). This prevents any possible extension of the ME or genomic fragment ends to generate priming sites for primers 1 and 3 during PCR, as well as preventing extension of the ends due to hybridization with repetitive sequences or short sequences with homology to the 3′ fragment ends. The other steps are the same as described above for FIG. 1.

In one embodiment, the sequence tag in the methods described above comprises uracil.

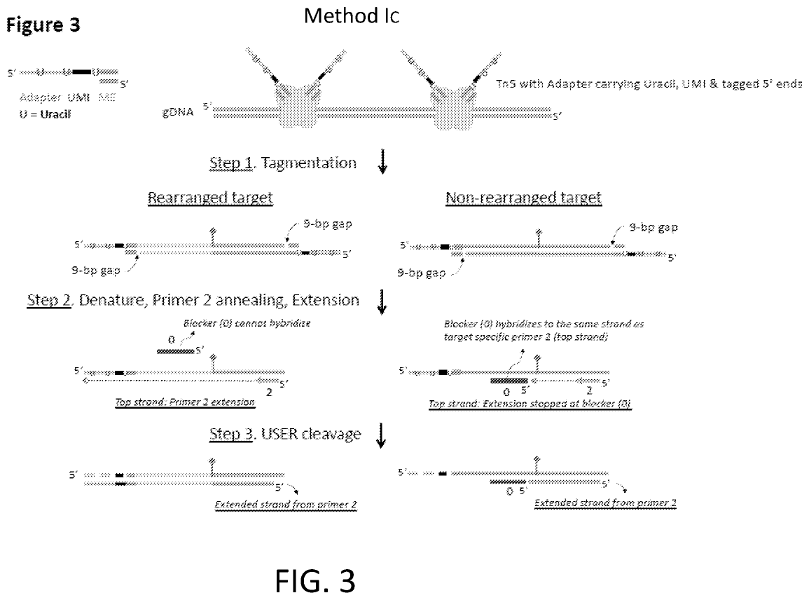

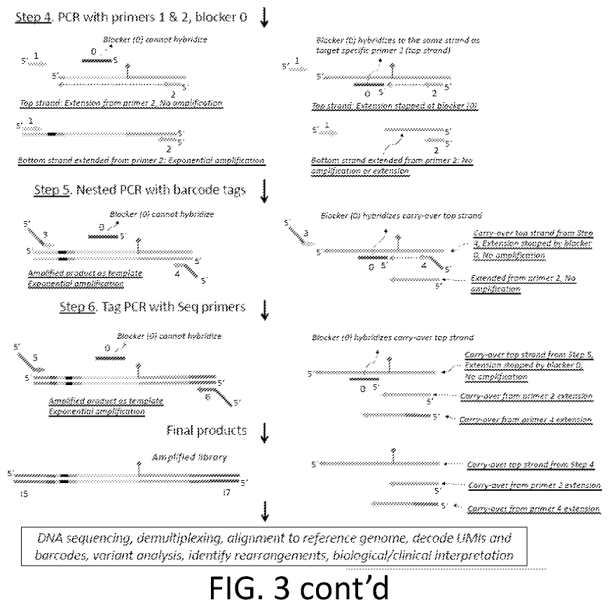

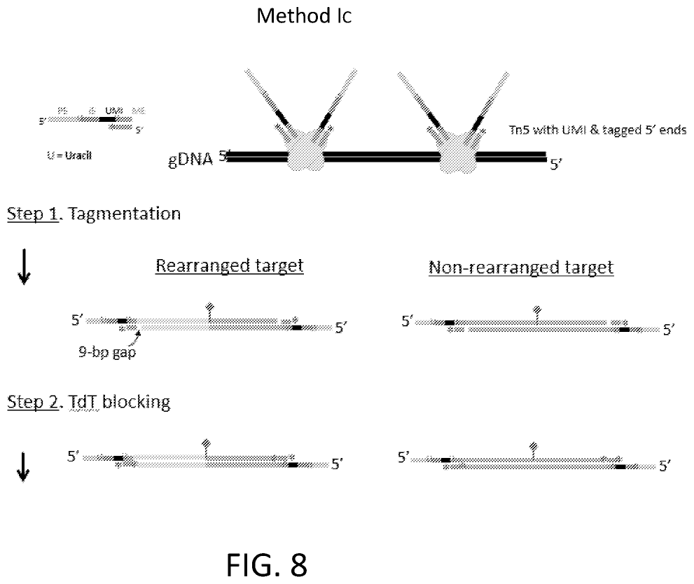

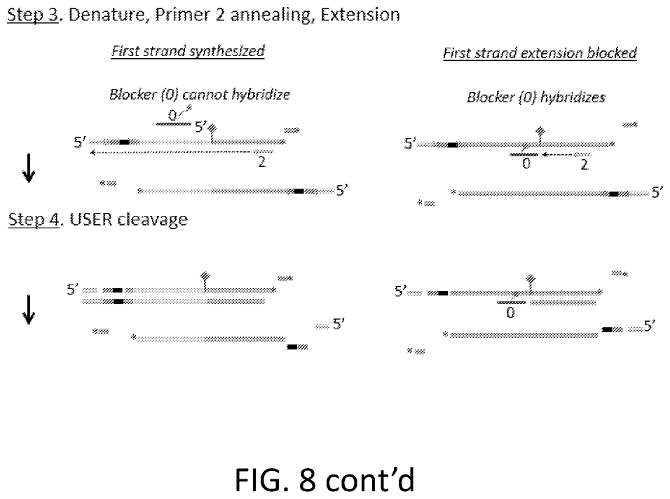

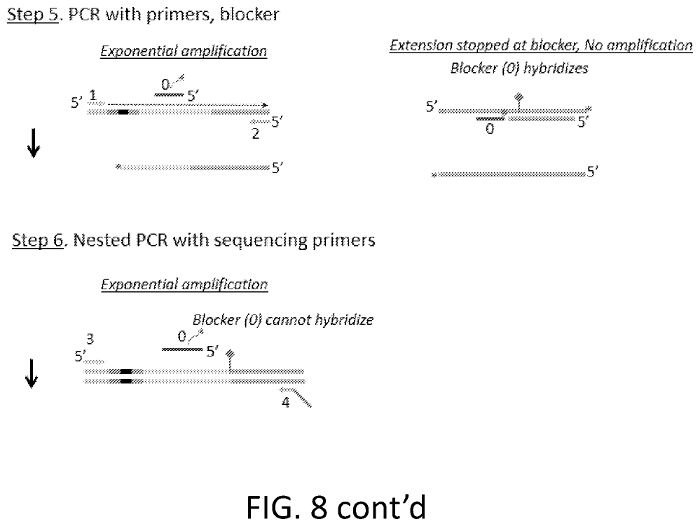

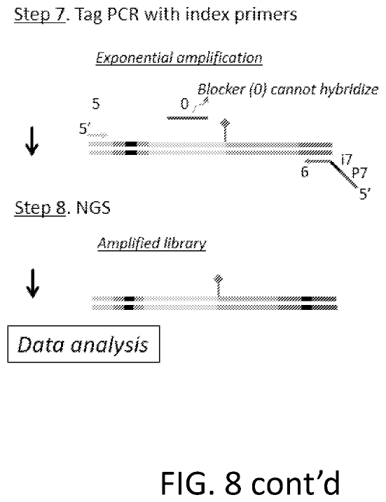

In accordance with this embodiment, an exemplary embodiment of the method described herein is shown in FIG. 3 and FIG. 8 (Method I_{C}). As shown in FIG. 3 and FIG. 8, the sequence tag used to assemble transposome carries Uracil. Unlike other current protocols, there is no strand displacement step after tagmentation. Instead, DNA is purified, then a PCR reaction is set up with primer 2 and a uracil-tolerating DNA polymerase (like Phusion U Hot Start DNA Polymerase, Cat #F555S, Thermo Fisher Scientific). The extension from primer 2 using the non-rearranged DNA as template is blocked by the blocker 0, while blocker 0 cannot block the extension using rearranged DNA as templates. This primer 2 extension step can be performed multiple times to generate multiple copies of the primer 2 extension strands. Alternatively, isothermal amplification method can be used to produce more copies, like Recombinase polymerase amplification (RPA) and strand-invasion based amplification (SIBA). Next, purified DNA is treated with USER (Uracil-Specific Excision Reagent) Enzyme (a mixture of Uracil DNA glycosylase (UDG) and DNA glycosylase-lysase Endonuclease VIII) at uracil residues synthetically incorporated into the adapter. In some embodiments, USER enzyme cleavage can performed by mixing 0.5, 1, 2, 3, 4, 5, 6, 7, 8, 9, or 10 μL of USER enzyme with product from the previous step, and incubating at 16, 20, 25, 30, 35, 40, or 45° C. for 5, 10, 15, 20, 25, 30, 35, 40, 45, 50, 55, or 60 minutes. The following steps of FIG. 3 and FIG. 8 are the same as FIG. 1 from Step 2 onwards.

### Method II

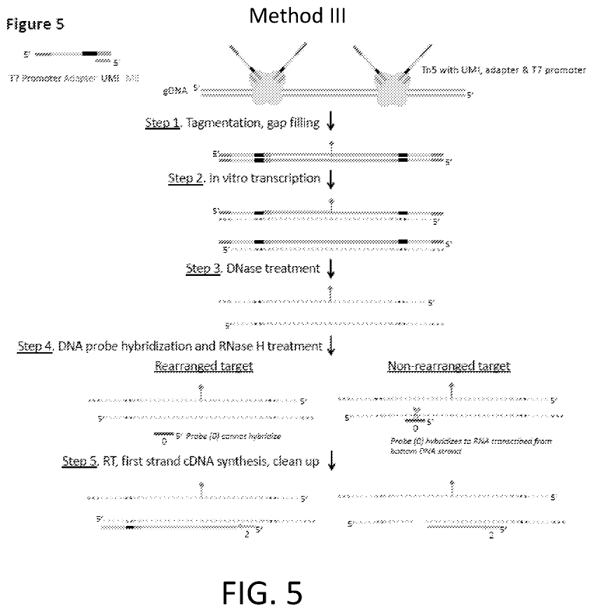

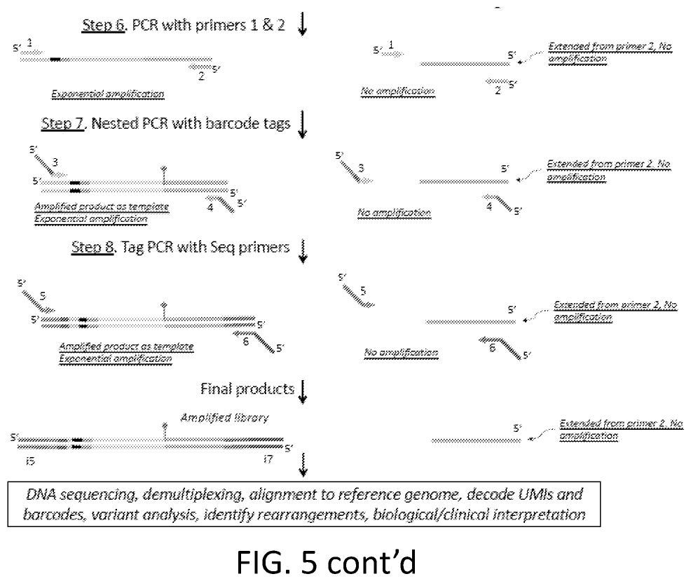

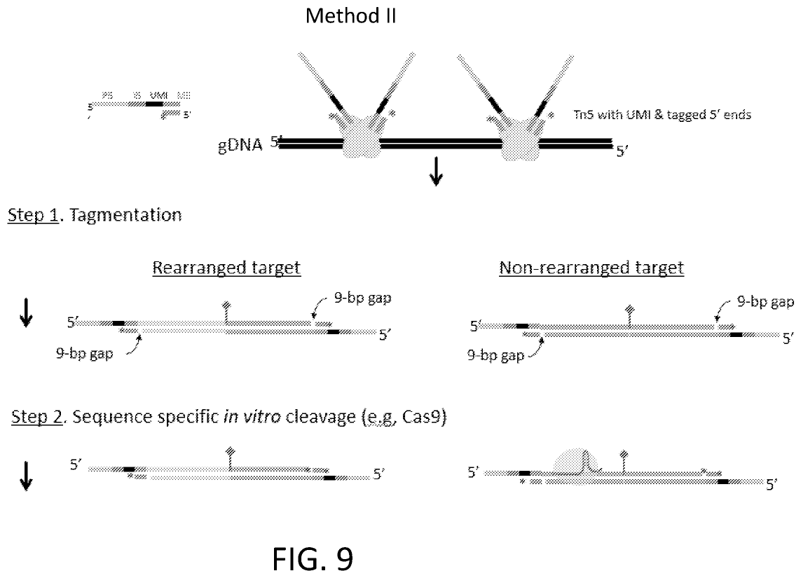

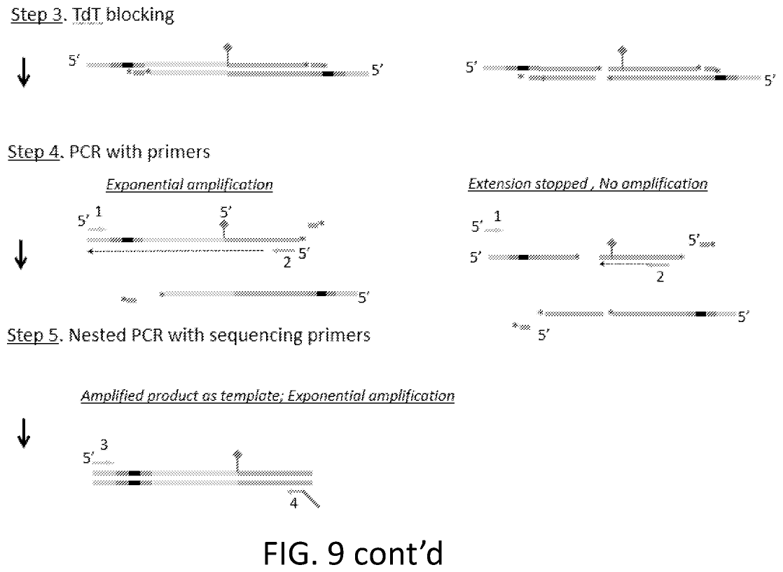

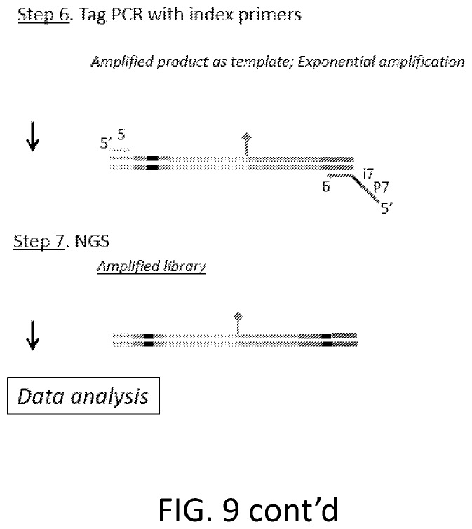

The present disclosure also includes another method for detecting genome-wide re-arrangements in a nucleic acid genome. The method includes (a) contacting a genomic DNA sample obtained from a cell or tissue contacted with a site-specific nuclease with a plurality of transposons comprising a first sequence tag at the 5′ end of the transposon, under conditions whereby the plurality of transposons is inserted into the genomic DNA sample and the genomic DNA sample is tagmented into a plurality of nucleic acid fragments each comprising the sequence tag at the 5′ ends of a partially double-stranded nucleic acid fragment. In some embodiments, the sequence tag comprises a sequence orthogonal to the genome, a UMI, and transposase recognition site (FIG. 5). In some embodiments, the sequence tag comprises a sequence orthogonal to the genome, a UMI, an index sequence, and transposase recognition site (FIG. 9). The method then includes (b) contacting the plurality of tagmented nucleic acid fragments with a sequence specific cleavage reagent; (c) performing a first amplification reaction to amplify the re-arranged tagmented nucleic acid fragments using (i) a first target-specific primer comprising a nucleotide sequence complementary to a region adjacent to a target site, (ii) a second primer comprising a nucleotide sequence identical to a portion of the sequence tag located at the 5′ ends of the fragment and; (d) sequencing the primary amplification products.

Methods and embodiments of step (a) are described above for Method I. However, unlike the methods described supra, which utilize blocking oligonucleotides, the method in the accordance with this aspect of the disclosure involves contacting the plurality of tagmented nucleic acid fragments with a sequence specific cleavage reagent.

### Sequence Specific Cleavage Agent

In one embodiment, the sequence specific cleavage reagent is an enzymatic reagent, or a chemical cleavage agent.

In methods presently disclosed herein, contacting step(s) with the nuclease are generally carried under out under conditions favorable for the cleavage by the nuclease. That is, even though a given candidate target site or variant target site might not actually be cleaved by the nuclease, the incubation conditions are such that the nuclease would have cleaved at least a significant portion (e.g., at least 10%, at least 20%, at least 30%, at least 40%, at least 50%, at least 60%, at least 70%, at least 80%, at least 90%, at least 95%, or at least 99%) of templates containing its known target site. For known and generally well-characterized nucleases, such conditions are generally known in the art and/or can easily be discovered or optimized. For newly discovered nucleases, such conditions can generally be approximated using information about related nucleases that are better characterized (e.g., homologs and orthologs).

In some embodiments, the nuclease is an endonuclease. In some embodiments, the nuclease is a site-specific endonuclease (e.g., a restriction endonuclease, a meganuclease, a transcription activator-like effector nucleases (TALEN), a zinc finger nuclease, etc.).

In some embodiments, the site specificity of a site-specific nuclease is conferred by an accessory molecule. For example, the CRISPR-associated (Cas) nucleases are guided to specific sites by “guide RNAs” or gRNAs as described herein. In some embodiments, the nuclease is an RNA-guided nuclease. In some embodiments, the nuclease is a CRISPR-associated nuclease.

In some embodiments, the nuclease is a homolog or an ortholog of a previously known nuclease, for example, a newly discovered homolog or ortholog.

RNA-guided nucleases according to the present disclosure include, but are not limited to, naturally-occurring Class 2 CRISPR nucleases such as Cas9, and Cas12a, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below.

In one embodiment, the enzymatic cleavage agent comprises a CRISPR Cas/gRNA complex.

In one embodiment, a guide RNA homologous to a blocking oligonucleotide location can be used in conjunction with a Cas nuclease (e.g., SpCas9) to cleave the tagmented genomic DNA. This would selectively prevent amplification of native, non-rearranged fragments, while allowing rearranged loci (un-cleaved) to be amplified in exactly the same manner as in the blocking oligonucleotide procedure.

In one embodiment, the sequence specific cleavage reagent is a chemical cleavage agent. Chemical cleavage can encompass any method which utilizes a non-nucleic acid and non-enzymatic chemical reagent in order to promote/achieve cleavage of one or both strands of a double-stranded nucleic acid molecule. If required, one or both strands of the double-stranded nucleic acid molecule may include one or more non-nucleotide chemical moieties and/or non-natural nucleotides and/or non-natural backbone linkages in order to permit chemical cleavage reaction.

In one embodiment, the chemical cleavage agent can be linked to an oligonucleotide. Exemplary chemical cleavage agents linked to oligonucleotides that can be useful in the method herein include, without limitation, chelated metal ions (e.g., Ag, Cu, Fe).

In one embodiment, the sequence tag comprises a sequence orthogonal to the genome, a unique molecular identifier (UMI), a transposase recognition site, an index sequence, or a combination thereof as described above for Method I.

In one embodiment, the first and second primers comprise second and third sequence tags as described above for Method I.

As described above for Method I, the first amplification round can be followed by a second amplification round. Thus, in some embodiments, the second amplification includes nested PCR and the introduction of second and third sequence tags. In some embodiments, the second amplification includes hemi-nested PCR.

In one embodiment, where a second amplification round is used, the method disclosed herein can further include prior to (d) performing a second amplification reaction using third and fourth nested primers comprising nucleotide sequences complementary to sequences proximal to the inside ends of the amplified products of (c) and additional sequence tags at the 5′ ends of the nested primers, to produce secondary amplification products comprising a re-arranged target sequence and additional sequence tags. In one embodiment, the method further includes performing a third amplification reaction using a fifth and sixth primer comprising nucleotide sequences complementary to the amplified products of the second amplification reaction to produce further enriched tertiary amplification products comprising a re-arranged target sequence.

In one embodiment, where a second amplification round is used, the method disclosed herein can further include prior to (d) performing a second, hemi-nested amplification reaction using the second primer and a third nested primer wherein the third primer comprises a nucleotide sequence complementary to a sequence proximal to target-specific primer end of the amplified products of (c) and optionally a fourth and fifth sequence tags at the 5′ ends of the second and/or third primers to produce secondary amplification products comprising a re-arranged target sequence and one or two additional sequence tags. In one embodiment, the method further includes performing a third amplification reaction using a fourth and fifth primer comprising nucleotide sequences complementary to the amplified products of the second amplification reaction to produce further enriched tertiary amplification products comprising a re-arranged target sequence.

In one embodiment, the products of the first amplification reaction are subjected to a repeated cleavage reaction before proceeding with the second and/or third amplification reactions, respectively.

In one embodiment, the third and/or the fourth primers include barcode sequences as described above for Method I.

In one embodiment, the fifth and sixth primers include a sequencing tag and/or an index sequence as described above for Method I.

In one embodiment, the method further includes performing a separate amplification reaction to amplify the re-arranged tagmented nucleic acid fragments using a second target specific primer on the opposite side of the target site.

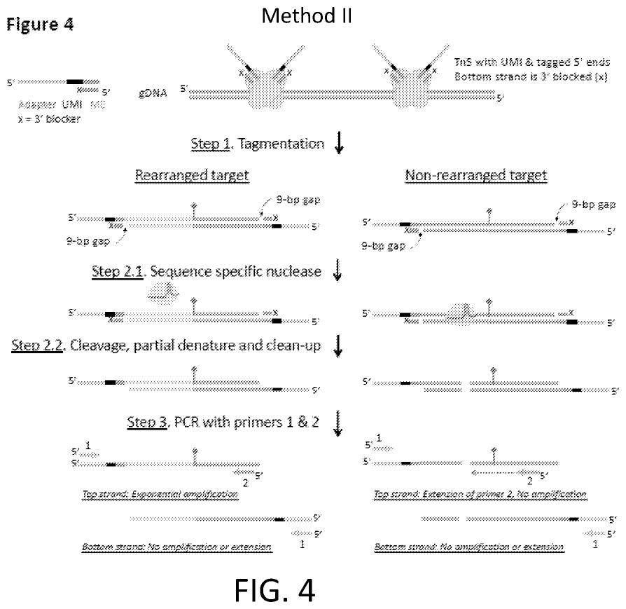

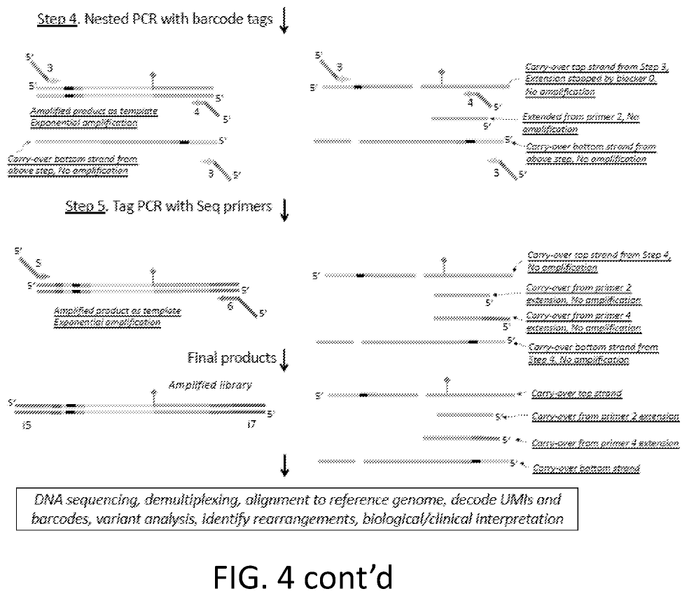

Exemplary embodiments of the above method described herein is shown in FIG. 4 and FIG. 9. As shown in FIG. 4 and FIG. 9, instead of using blocking oligos as in Method I, Method II employs sequence specific cleavage using CRISPR/Cas, TALEN, ZFN, or similar sequence directed enzymatic cleavage reagents. In some embodiments, cleavage reactions can be performed by mixing a guide RNA with a Cas9 nuclease at 15, 20, 25, 30, 35, 40, or 45° C. for 1, 10, 20, 30, 40, 50, 60, 70, 80, 90, 100, 110, 120 minutes.

Another alternative might be the use of a chemical cleavage reagent linked to an oligonucleotide. This would selectively prevent amplification of native, non-rearranged fragments, while allowing rearranged loci (un-cleaved) to be amplified in exactly the same manner as in the blocking oligo procedure. After the cleavage reaction is completed, all 3′ ends are blocked by treatment with ddNTP and terminal deoxynucleotidyl transferase (TdT), Klenow exo-, or another suitable enzyme (TdT has the advantage that it can incorporate 3′ blocked nucleotides at staggered or blunt ends). End-blocking will reduce the generation of interfering side products in the subsequent PCR reactions.

### Method III

The present disclosure is also directed to a method for detecting genome-wide re-arrangements in a nucleic acid genome. The method includes (a) contacting a genomic DNA sample obtained from a cell or tissue contacted with a site specific nuclease with a plurality of transposons comprising a first sequence tag at the 5′ end of the transposon, wherein the sequence tag comprises an RNA promoter sequence, under conditions whereby the plurality of transposons is inserted into the genomic DNA sample and the genomic DNA sample is tagmented into a plurality of nucleic acid fragments each comprising the sequence tag at the 5′ ends of a partially double-stranded nucleic acid fragment; (b) transcribing the plurality of tagmented nucleic acid fragments into RNA; (c) contacting the RNA with target sequence specific DNA oligonucleotide probes and RNase H; (d) performing a reverse transcription amplification reaction using a (i) a first primer comprising a nucleotide sequence complementary to a portion of the sequence tag located at the 3′ or 5′ end of the fragment and (ii) a second primer comprising a nucleotide sequence complementary to a region adjacent to a target site to produce primary amplification products; (e) sequencing the primary amplification products.

### RNA Promoter Sequence

RNA promoter sequences useful in the method described herein are sequences capable of binding an RNA polymerase and contain a transcriptional start site. Accordingly, the promotor sequence usually includes between about 15 and about 250 nucleotides, preferably between about 25 and about 60 nucleotides, from a naturally occurring RNA polymerase promoter, a consensus promoter sequence (Alberts et al., in Molecular Biology of the Cell, 2d Ed., Garland, N.Y. (1989), or a modified version thereof. Exemplary RNA promoters are the T3, T7, and SP6 phage promoter/polymerase systems.

Unlike previous methods described herein, this aspect of the disclosure incudes the steps of (b) transcribing the plurality of tagmented nucleic acid fragments into RNA; (c) contacting the RNA with target sequence specific DNA oligonucleotide probes and RNase H; (d) performing a reverse transcription amplification reaction. The earlier and later steps of the method are similar to those described for the other methods above.

In one embodiment, the sequence tag comprises a sequence orthogonal to the genome, a unique molecular identifier (UMI), a transposase recognition site, an index sequence, or a combination thereof as described above.

In one embodiment, the first and second primers comprise sequence tags as described above.

An exemplary embodiment of the above method described herein is shown in FIG. 5. As shown in FIG. 5, the special endoribonuclease activity of RNase H (Ribonuclease H), which specifically hydrolyzes the phosphodiester bonds of RNA when hybridized to DNA. In this method, T7 promoter (or other in vitro transcription promoter) is included in the transposon adapter. After tagmentation, DNA is in vitro transcribed into RNA. In some embodiments, the in vitro transcription reaction can be performed using MAXIscript™ T7 Transcription Kit and incubating at 25, 30, 35, 40, or 45° C. for 1, 10, 20, 30, 40, 50, 60, 70, 80, 90, 100, 200, 300, 400, 500, or 600 minutes.

Then sequence specific DNA probes are hybridized to the RNA and treated with RNase H, which will specifically cleave the RNA region hybridized with the DNA probes. Then, one-step Reverse Transcription PCR is performed using sequence specific primer 2. In some embodiments, Primer 2 is designed specific to the right-side sequence of potential rearrangement site, and design the DNA probe specific to the left-side sequence of the same site. Thus, the non-rearranged templates will be cleaved by RNase H and cannot be amplified by primers 1 and 2, while the rearranged templates are not cleaved by the DNA probes and can be exponentially amplified. In some embodiments, for DNA probe hybridization and RNase H treatment, reactions are performed in two separate reactions for each target, one reaction for detecting genome rearrangement on the left-side of the target and the other reaction for detecting genome rearrangement on the right-side of the target. In some embodiments, the reaction is denatured at about 85, 86, 87, 88, 89, 90, 91, 92, 93, 94, 95, 96, 97, 98, or 99° C. for about 1, 2, 3, 4, 5, 6, 7, 8, 9, or 10 minutes. In some embodiments, RNase H is added to the reaction and incubated at about 16, 20, 25, 30, 35, 40, or 45° C. for about 1, 10, 20, 30, 40, 50, 60, 70, 80, 90, 100, 200 or 300 minutes. In some embodiments, RNase H is inactivated at 60, 65, 70, 75, 80, 85, or 90° C. for about 5, 10, 20, 30, 40, 50, 60, 70, 80, 90, 100, 110, 120, 130, 140, 150, 160, 170, 180, 190, 200, 210, 220, or 240 minutes. After first strand synthesis, the following steps are the same as in FIG. 4 from Step 3 onwards.

In one embodiment, the method further can include prior to (e) performing a second amplification reaction using third and fourth nested primers comprising nucleotide sequences complementary to sequences proximal to the inside ends of the amplified products of (d) and additional sequence tags, to produce secondary amplification products comprising a re-arranged target sequence and additional sequence tags. In one embodiment, the method further includes performing a third amplification reaction using a fifth and sixth primer comprising nucleotide sequences complementary to the amplified products of the second amplification reaction to produce further enriched tertiary amplification products comprising a re-arranged target sequence.

In one embodiment, the method further can include prior to (e) performing a second, hemi-nested amplification reaction using the second primer and a third nested primer wherein the third primer comprises a nucleotide sequence complementary to a sequence proximal to target-specific primer end of the amplified products of (d) and optionally a fourth and fifth sequence tags at the 5′ ends of the second and/or third primers to produce secondary amplification products comprising a re-arranged target sequence and one or two additional sequence tags. In one embodiment, the method further includes performing a third amplification reaction using a fourth and fifth primer comprising nucleotide sequences complementary to the amplified products of the second amplification reaction to produce further enriched tertiary amplification products comprising a re-arranged target sequence.

In one embodiment, the third and/or fourth primers include barcode sequences as described above.

In one embodiment, the fifth and sixth primers comprise an adapter sequence and/or an index sequence as described above.

In one embodiment, the method further includes wherein (d) further comprises performing a separate amplification reaction to amplify the re-arranged tagmented nucleic acid fragments using a second target specific primer on the opposite side of the target site.

### Data Analysis

For the methods described herein, data analysis can be performed using paired-end sequencing reads from Illumina sequencing that are merged using a paired-end reads merging software, e.g., PEAR (Zhang et al, Bioinformatics. 2014; 30:614-620), FLASH (Magoč and Salzberg, Bioinformatics. 2011; 27:2957-2963), BBMerge (Bushnell et al, PLoS One. 2017; 12:e0185056), or NGmerge (Gaspar, Bioinformatics. 2018; 19:536). The merged reads can be trimmed and filtered using software such as BBmap (https://jgi.doe.gov/data-and-tools/software-tools/bbtools/), samtools (Danecek et al, Gigascience. 2021; 10. doi: 10.1093/gigascience/giab008), or custom scripts to remove Illumina adaptor sequences, low quality reads, reads containing the target flanking sequence on the side of the target site where the blocking oligonucleotide or cleavage reagent binds, and reads that do not contain the target flanking sequence on the side of the target site where the target-specific primer hybridizes. Selected reads can be aligned to a human reference genome, e.g., GRCh37 (hg19), GRCh38 (hg38), or Telomere-to-Telomere assembly (e.g., T2T-CHM13v2.0), using an alignment software, e.g., Bowtie 2 (Langmead and Salzberg, Nat Methods. 2012; 9:357-359), BWA (Li and Durbin, Bioinformatics. 2009; 25:1754-1760), or Minimap2 (Li, Bioinformatics. 2018; 34:3094-3100). The aligned BAM file can be converted into bed file using BEDTools (Quinlan, Bioinformatics. 2014; 47:11.12.1-34). For methods using UMIs, UMIs can be collapsed to remove redundant sequencing reads using software such as Gencore (Chen et al, Bioinformatics. 2019; 20:606), UMI-tools (Smith et al, Genome Res. 2017:27: 491-499), UMI-Reducer (/github.com/smangul1/UMI-Reducer), or custom scripts.

Reads with candidate translocation break points within a suitable window flanking the target site (e.g., within 1, 3, 5, 10, 20, 40, 60, 80, 100, 200, or more bases) can be identified and counted to quantify the number of rearrangements between the target site and other genomic loci. Statistical tests can be applied to establish a confidence score to each group of rearrangements at each distal rearranged locus (e.g., within a defined window of up 50, 100, 200, 500, 1000, 2000, 3000, or more bases at the distal rearranged locus). Visualization tools such as IGV (Thorvaldsdóttir et al, Brief Bioinform. 2013; 14:178-192) or Circos (Krzywinski et al, Genome Res. 2009; 19:1639-1645) can be used to facilitate the identification and analysis of rearrangements at genomic scale.

### Systems and Kits

Also provided herein are systems and kits including the reagents needed for performing the methods described herein as well as written instructions for making and using the same.

Any of the above-described systems and kits can further include one or more additional reagents.

In some embodiments, a system or kit can further include instructions for using the components of the kit to practice the methods. The instructions for practicing the methods are generally recorded on a suitable recording medium. For example, the instructions can be printed on a substrate, such as paper or plastic, and the like. The instructions can be present in the kits as a package insert, in the labeling of the container of the kit or components thereof (i.e., associated with the packaging or sub-packaging), and the like. The instructions can be present as an electronic storage data file present on a suitable computer readable storage medium, e.g., CD-ROM, diskette, flash drive, and the like. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### EXAMPLES

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### Example 1: Exemplary Method for Method I_{A}

Exemplary oligonucleotides for use in the methods are listed in Table 1.

TABLE 1
Exemplary oligonucleotide sequences.

| | | | | Coordinate | Method | Method | Method | Method | Method |
|---|---|---|---|---|---|---|---|---|---|
| | | | | in hg38 | I_{A} | I_{B} | I_{C} | II | III |
| Line_ | | | | (chr strand | Example 1 | Example 3 | Example 5 | Example 7 | Example 9 |
| ID | Name | Sequence and modification | Note | start end) | FIG. 1 | FIG. 2 | FIG. 3 | FIG. 4 | FIG. 5 |
| Oligo- | Tn5 | CGTACCGATACGCTCGGACAGATGTGTATAAG | Common | | Yes | Yes | No | Yes | No |
| 01 | adapter | AGACAG (SEQ ID NO: 1) | | | | | | | |
| | top | | | | | | | | |
| Oligo- | Tn5 | CTGTCTCTTATACACATCT | Common | | Yes | Yes | Yes | No | Yes |
| 02 | adapter | (SEQ ID NO: 2) | | | | | | | |
| | bottom | | | | | | | | |
| Oligo- | Blocker | +T+C+CTCCAG+CTTCT+GCCGT+T+T+G/ | target specific, for | chr2 − | Yes | Yes | Yes | No | No |
| 03 | 0- | 3AmMO/ | EMX1 Left side | 72933822 | | | | | |
| | EMX1_L | (SEQ ID NO: 3) | rearrangement | 72933842 | | | | | |
| Oligo- | Primer 1 | GCGTACCGATACGCTCGGAC (SEQ ID | Common | | Yes | Yes | Yes | Yes | Yes |
| 04 | | NO: 4) | | | | | | | |
| Oligo- | Primer | GGTTTGTGGTTGCCCACCCT (SEQ ID | target specific, for | chr2 − | Yes | Yes | Yes | Yes | Yes |
| 05 | 2- | NO: 5) | EMX1 Left side | 72933953 | | | | | |
| | EMX1_L | | rearrangement | 72933972 | | | | | |
| Oligo- | Primer 3 | ACACTCTTTCCCTACACGACGCTCTTCCGATC | Common | | Yes | Yes | Yes | Yes | Yes |
| 06 | | TCGTACCGATACGCTCGGAC (SEQ ID | | | | | | | |
| | | NO: 6) | | | | | | | |
| Oligo- | Primer | GTGACTGGAGTTCAGACGTGTGCTCTTCCGAT | target specific, for | chr2 − | Yes | Yes | Yes | Yes | Yes |
| 07 | 4- | CTCCTCCCCATTGGCCTGCTTC (SEQ ID | EMX1 Left side | 72933909 | | | | | |
| | EMX1_L | NO: 7) | rearrangement | 72933928 | | | | | |
| Oligo- | Primer 5 | AATGATACGGCGACCACCGAGATCTACACTAT | common, bold is | | Yes | Yes | Yes | Yes | Yes |
| 08 | | AGCCTACACTCTTTCCCTACACGACGCTCTTC | index, this is an | | | | | | |
| | | CGATC*T (SEQ ID NO: 8) | example of NEBNext | | | | | | |
| | | | i501 Primer | | | | | | |
| Oligo- | Primer 6 | CAAGCAGAAGACGGCATACGAGATCGAGTAA | common, bold is | | Yes | Yes | Yes | Yes | Yes |
| 09 | | TGTGACTGGAGTTCAGACGTGTGCTCTTCCGA | index, this is an | | | | | | |
| | | TC*T (SEQ ID NO: 9) | example of NEBNext | | | | | | |
| | | | i701 Primer | | | | | | |
| Oligo- | Tn5 | CGTACCGAUACGCTCGGACUAGATGTGTATAA | Common | | No | No | Yes | No | No |
| 10 | adapter | GAGACAG (SEQ ID NO: 10) | | | | | | | |
| | top U | | | | | | | | |
| Oligo- | Tn5 | CTGTCTCTTATACACATCT/3ddC/ | Common | | No | No | No | Yes | No |
| 11 | adapter | (SEQ ID NO: 11) | | | | | | | |
| | bottom | | | | | | | | |
| | 3′ | | | | | | | | |
| | blocked | | | | | | | | |
| Oligo- | Tn5 | TAATACGACTCACTATAGCGTACCGATACGCT | Common | | No | No | No | No | Yes |
| 12 | adapter | CGGACAGATGTGTATAAGAGACAG (SEQ ID | | | | | | | |
| | top T7p | NO: 12) | | | | | | | |
| Oligo- | Probe 0- | TCCTCCAGCTTCTGCCGTTTG (SEQ ID | target specific, for | chr2 − | No | No | No | No | Yes |
| 13 | EMX1_L | NO: 13) | EMX1 Left side | 72933822 | | | | | |
| | | | rearrangement | 72933842 | | | | | |
| Oligo- | Blocker | +C+G+AAGCA+GGCCA+ATGGG+G+A+G/ | target specific, for | chr2 + | Yes | Yes | Yes | No | No |
| 14 | 0- | 3AmMO/ (SEQ ID NO: 14) | EMX1 Right side | 72933908 | | | | | |
| | EMX1_R | | rearrangement | 72933927 | | | | | |
| Oligo- | Primer | TCTCATCTGTGCCCCTCCCT (SEQ ID | target specific, for | chr2 + | Yes | Yes | Yes | Yes | Yes |
| 15 | 2- | NO: 15) | EMX1 Right side | 72933756 | | | | | |
| | EMX1_R | | rearrangement | 72933775 | | | | | |
| Oligo- | Primer | GTGACTGGAGTTCAGACGTGTGCTCTTCCGAT | target specific, for | chr2 + | Yes | Yes | Yes | Yes | Yes |
| 16 | 4- | CTGTGTGGTTCCAGAACCGGAGG (SEQ ID | EMX1 Right side | 72933793 | | | | | |
| | EMX1_R | NO: 16) | rearrangement | 72933813 | | | | | |
| Oligo- | Probe | CGAAGCAGGCCAATGGGGAG (SEQ ID NO: | target specific, for | chr2 + | No | No | No | No | Yes |
| 17 | 0- | 17) | EMX1 Right side | 72933908 | | | | | |
| | EMX1_R | | rearrangement | 72933927 | | | | | |
| Oligo- | Blocker | +A+G+GGCTCC+GATGTA+TAATAA+T+T+G/ | target specific, for | chr3 − | Yes | Yes | Yes | No | No |
| 18 | 0- | 3AmMO/ | CCR5#1 Left side | 46372938 | | | | | |
| | CCR5#1_ | (SEQ ID NO: 18) | rearrangement | 46372960 | | | | | |
| | L | | | | | | | | |
| Oligo- | Primer | AGGTAGATGTCAGTCATGCT (SEQ ID | target specific, for | chr3 − | Yes | Yes | Yes | Yes | Yes |
| 19 | 2- | NO: 19) | CCR5#1 Left side | 46373089 | | | | | |
| | CCR5#1_ | | rearrangement | 46373108 | | | | | |
| | L | | | | | | | | |
| Oligo- | Primer | GTGACTGGAGTTCAGACGTGTGCTCTTCCGAT | target specific, for | chr3 − | Yes | Yes | Yes | Yes | Yes |
| 20 | 4- | CTGCTCTTCAGCCTTTTGCAGTTTATCAG | CCR5#1 Left side | 46373065 | | | | | |
| | CCR5#1_ | (SEQ ID NO: 20) | rearrangement | 46373091 | | | | | |
| | L | | | | | | | | |
| Oligo- | Probe | AGGGCTCCGATGTATAATAATTG (SEQ | target specific, for | chr3 − | No | No | No | No | Yes |
| 21 | 0- | ID NO: 21) | CCR5#1 Left side | 46372938 | | | | | |
| | CCR5#1_ | | rearrangement | 46372960 | | | | | |
| | L | | | | | | | | |
| Oligo- | Blocker | +G+G+CAACAT+GCTGGT+CATCC+T+C+A/ | target specific, for | chr3 + | Yes | Yes | Yes | No | No |
| 22 | 0- | 3AmMO/ (SEQ ID NO: 22) | CCR5#1 Right side | 46373041 | | | | | |
| | CCR5#1_ | | rearrangement | 46373062 | | | | | |
| | R | | | | | | | | |
| Oligo- | Primer | ACAGGGTGGAACAAGATGGAT (SEQ ID | target specific, for | chr3 + | Yes | Yes | Yes | Yes | Yes |
| 23 | 2- | NO: 23) | CCR5#1 Right side | 46372888 | | | | | |
| | CCR5#1_ | | rearrangement | 46372908 | | | | | |
| | R | | | | | | | | |
| Oligo | Primer | GTGACTGGAGTTCAGACGTGTGCTCTTCCGAT | target specific, for | chr3 + | Yes | Yes | Yes | Yes | Yes |
| 24 | 4- | CTTACATCGGAGCCCTGCCAAA (SEQ ID | CCR5#1 Right side | 46372947 | | | | | |
| | CCR5#1_ | NO: 24) | rearrangement | 46372966 | | | | | |
| | R | | | | | | | | |
| Oligo- | Probe | GGCAACATGCTGGTCATCCTCA (SEQ ID | target specific, for | chr3 + | No | No | No | No | Yes |
| 25 | 0- | NO: 25) | CCR5#1 Right side | 46373041 | | | | | |
| | CCR5#1_ | | rearrangement | 46373062 | | | | | |
| | R | | | | | | | | |
| Oligo- | Tn5 | CGTACCGATACGCTCGGACNNNNNNNNNNN | UMI version of | | Yes | Yes | No | Yes | No |
| 26 | adapter | NAGATGTGTATAAGAGACAG (SEQ ID | Oligo-01 | | | | | | |
| | top UMI | NO: 26) | | | | | | | |
| Oligo- | Tn5 | CGTACCGAUACGCTCGGACUNNNNNNNNNN | UMI version of | | No | No | Yes | No | No |
| 27 | adapter | NNUAGATGTGTATAAGAGACAG (SEQ | Oligo-10 | | | | | | |
| | top U | ID NO: 27) | | | | | | | |
| | UMI | | | | | | | | |
| Oligo- | Tn5 | TAATACGACTCACTATAGCGTACCGATACGCT | UMI version of | | No | No | No | No | Yes |
| 28 | adapter | CGGACNNNNNNNNNNNNAGATGTGTATAAGA | Oligo-12 | | | | | | |
| | top T7p | GACAG (SEQ ID NO: 28) | | | | | | | |
| | UMI | | | | | | | | |

For site specific oligos, the “R” or “L” in the oligo name is defined by which side the blocker/probe 0 is annealed to relative to the cleavage site, when the genomic DNA is kept its +/− strands orientation, despite the target sequence (PAM) is on (+) or (−) strand.

Exemplary oligonucleotide sets are shown in Table 2 and Table 3 below. For example, for EMX1 or CCR5 #1, a first reaction is performed to detect rearrangements on the left-side of the on-target cleavage site, and a second reaction is performed to detect rearrangements on the right-side of the on-target cleavage site. For each reaction, a set of target specific primers and blocker is to be used. For EMX1 left-side rearrangements, Oligos Mar. 5, 2007/13 are used, and for EMX1 right-side rearrangements, Oligos 14/15/16/16 are used. Similarly, for CCR5 #1 left-side, Oligos 18/19/20/21 are used and for CCR5 #1 right side, Oligos 22/23/24/25 are used.

TABLE 2
Target-specific primers and blocker

| | for EMX1 Left side | | for EMX1 Right side | | for CCR5#1 Left side | | for CCR5#1 Right | |
|---|---|---|---|---|---|---|---|---|
| | rearrangement | | rearrangement | | rearrangement | | side rearrangement | |
| Common | EMX1_L | | EMX1_R | | CCR5_L | | CCR5_R | |
| Name | Line_ID | Name | Line_ID | Name | Line_ID | Name | Line_ID | Name |
| Blocker 0 | Oligo-03 | Blocker 0 - | Oligo- | Blocker 0 - | Oligo- | Blocker 0 - | Oligo- | Blocker 0 |
| | | EMX1_L | 14 | EMX1_R | 18 | CCR5#1_L | 22 | CCR5#1 R |
| Primer 2 | Oligo-05 | Primer 2 - | Oligo- | Primer 2 - | Oligo- | Primer 2 - | Oligo- | Primer 2 - |
| | | EMX1_L | 15 | EMX1_R | 19 | CCR5#1_L | 23 | CCR5#1_R |
| Primer 4 | Oligo-07 | Primer 4 - | Oligo- | Primer 4 - | Oligo- | Primer 4 - | Oligo- | Primer 4 - |
| | | EMX1_L | 16 | EMX1_R | 20 | CCR5#1_L | 24 | CCR5#1_R |
| Probe 0 | Oligo-13 | Probe 0 - | Oligo- | Probe 0 - | Oligo- | Probe 0 - | Oligo- | Probe 0 - |
| | | EMX1_L | 17 | EMX1_R | 21 | CCR5#1_L | 25 | CCR5#1_R |

TABLE 3
Transposome oligonucleotides

| Non-UMI version | | UMI version | |
|---|---|---|---|
| Line_ID | Name | Line_ID | Name |
| Oligo-01 | Tn5 transposome top | Oligo-26 | Tn5 transposome top UMI |
| Oligo-10 | Tn5 transposome top U | Oligo-27 | Tn5 transposome top U UMI |
| Oligo-12 | Tn5 transposome top T7p | Oligo-28 | Tn5 transposome top T7p UMI |

Exemplary sgRNA target sequences are shown in Table 4 below.

TABLE 4

| | Sequence | Coordinate in hg38 |
|---|---|---|
| Name | (PAM is underlined) | (chr strand start end) |
| EMX1 target | GAGTCCGAGCAGAAGAAGAAGGG (SEQ | chr2 + 72933853 72933875 |
| sequence | ID NO: 29) | |
| CCR5#1 target | GTGAGTAGAGCGGAGGCAGGAGG (SEQ | chr3 − 46372995 46373017 |
| sequence | ID NO: 30) | |

Additional reagents include Tn5 transposase, e.g., Robust Tn5 Transposase (Cat #EMQZ1422, Creative Biogene), DNA polymerase PCR master mix, e.g., Platinum™ SuperFi II PCR Master Mix (ThermoFisher, Cat #12368010), DNA polymerase with PCR buffer, e.g., Platinum™ SuperFi II DNA Polymerase (ThermoFisher, Cat #12361010), dNTP, e.g., dNTP Mix (10 mM each) (ThermoFisher, Cat #R0194), DNA purification kit: Column kit, e.g., DNA Clean & Concentrator-5 (Zymo Research, Cat #D4004) Magnetic beads, e.g., AMPure XP Reagent (Beckman Coulter, Cat #A63880), Illumina sequencing kit, e.g., NextSeq 1000/2000 P1 Reagents (300 Cycles) (Cat #20050264), MiSeq Reagent Kit v3 (150-cycle) (Cat #MS-102-3001), MiSeq Reagent Kit v2 (300-cycles) (Cat #MS-102-2002), PhiX Control v3 (Illumina, Cat #FC-110-3001), STE buffer (10 mM Tris-HCl (pH 8.0), 1 mM EDTA, 0.1M NaCl).

In the first step, Tn5 transposomes are assembled. Transposon-end containing oligos (e.g., Oligo-01 and Oligo-02 for non-UMI version, or Oligo-26 and Oligo-02 for UMI version) are annealed in STE buffer with the following program: 1-10 minutes at 95-99° C., 40-500 cycles of 1 minute at 1-99° C. (decrease temperature 0.2-2° C. every cycle until reaching 1-12° C.). Transposome complexes are assembled in TPS buffer following the procedure below, as recommended by the manufacturer (Creative Biogene, Cat #EMQZ1422). The reagents are mixed thoroughly and incubated at 15-40° C. for 5-120 minutes.

| Component | Volume | |
|---|---|---|
| Tn5 transposase | 1-10 | μL |
| Adaptor | 0.5-8 | μL |
| 10x TPS buffer | 2 | μL |
| Sterile water | add to 20 | μL |

The workflow for this example is shown in FIG. 1. The tagmentation reaction is performed in which genomic DNA extracted from gene edited samples or controls (using standard methods) is tagmented with the assembled transposome complex following procedure. An exemplary reaction mixture is shown below:

| Component | Volume | |
|---|---|---|
| Genomic DNA | varies (5-500 ng) | |
| 5x LM buffer | 6 | μL |
| Transposome | 0.1-8 | μL |
| Sterile water | add to 30 | μL |

The reaction components are mixed thoroughly and incubated at 30-65° C. for 5-120 minutes. The tagmented DNA is purified using column-based purification.

Tagmented DNA from the previous step is used for two separate PCR reactions for each target, one reaction to detect genome rearrangement on the left-side of the target and the other reaction to detect genome rearrangement on the right-side of the target. Each PCR is then performed with a common primer annealed to the adapter region, a target specific blocker and a target specific primer. PCR is performed using 3-step PCR cycling protocol with tagmented DNA, Primer 1 (Oligo-04, common to all targets and rearrangement of both sides), Primer 2 (e.g., Oligo-05 for EMX1 left-side rearrangement, Oligo-15 for EMX1 right-side rearrangement, Oligo-19 for CCR5 #1 left-side rearrangement, or Oligo-23 for CCR5 #1 right-side rearrangement), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: initial denaturing at 90-99° C. for 1-5 minute, 20-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Nested PCR is then performed with barcode tag. PCR is performed with PCR product from the previous step, Primer 3 (Oligo-06, common to all targets and rearrangement of both sides), Primer 4 (e.g., Oligo-07 for EMX1 left-side rearrangement, Oligo-16 for EMX1 right-side rearrangement, Oligo-20 for CCR5 #1 left-side rearrangement, or Oligo-24 for CCR5 #1 right-side rearrangement), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: initial denaturing at 90-99° C. for 1-5 minute, 4-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Tag PCR is then performed with sequencing primers. PCR is performed with PCR product from the previous step, Primer 5 (e.g., Oligo-08, an example of Illumina indexing primer from New England Biolabs, NEB #E7603A), Primer 6 (e.g., Oligo-09, an example of Illumina indexing primer from New England Biolabs, NEB #E7611A), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: initial denaturing at 90-99° C. for 1-5 minute, 4-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Next generation sequencing is then performed using the PCR product from the previous step, and sequencing is performed using an Illumina platform (e.g., Illumina MiSeq or NextSeq 2000) with optional >=0-95% PhiX (Illumina, Cat #FC-110-3001).

Paired-end sequencing reads from Illumina sequencing are merged using a paired-end reads merging software, e.g., PEAR (Zhang et al, Bioinformatics. 2014; 30:614-620), FLASH (Magoč and Salzberg, Bioinformatics. 2011; 27:2957-2963), BBMerge (Bushnell et al, PLoS One. 2017; 12:e0185056), or NGmerge (Gaspar, Bioinformatics. 2018; 19:536). The merged reads are then trimmed and filtered using software such as BBmap (https://jgi.doe.gov/data-and-tools/software-tools/bbtools/), samtools (Danecek et al, Gigascience. 2021; 10. doi: 10.1093/gigascience/giab008), or custom scripts to remove Illumina adaptor sequences, low quality reads, reads containing the target flanking sequence on the side of the target site where the blocking oligonucleotide or cleavage reagent binds, and reads that do not contain the target flanking sequence on the side of the target site where the target-specific primer hybridizes. Selected reads are aligned to a human reference genome, e.g., GRCh37 (hg19), GRCh38 (hg38), or Telomere-to-Telomere assembly (e.g., T2T-CHM13v2.0), using an alignment software, e.g., Bowtie 2 (Langmead and Salzberg, Nat Methods. 2012; 9:357-359), BWA (Li and Durbin, Bioinformatics. 2009; 25:1754-1760), or Minimap2 (Li, Bioinformatics. 2018; 34:3094-3100). The aligned BAM file is converted into bed file using BEDTools (Quinlan, Bioinformatics. 2014; 47:11.12.1-34). For methods using UMIs, UMIs are collapsed to remove redundant sequencing reads using software such as Gencore (Chen et al, Bioinformatics. 2019; 20:606), UMI-tools (Smith et al, Genome Res. 2017; 27:491-499), UMI-Reducer (github.com/smangul1/UMI-Reducer), or custom scripts. Reads with candidate translocation break points within a suitable window flanking the target site (e.g., within 1, 3, 5, 10, 20, 40, 60, 80, 100, 200, or more bases) are identified and counted to quantify the number of rearrangements between the target site and other genomic loci. Statistical tests are then applied to establish a confidence score to each group of rearrangements at each distal rearranged locus (e.g., within a defined window of up 50, 100, 200, 500, 1000, 2000, 3000, or more bases at the distal rearranged locus). Visualization tools such as IGV (Thorvaldsdóttir et al, Brief Bioinform. 2013; 14:178-192) or Circos (Krzywinski et al, Genome Res. 2009; 19:1639-1645) are used to facilitate the identification and analysis of rearrangements at genomic scale.

### Example 2: Exemplary Method for Method I_{A}

Exemplary oligonucleotides used in the method are listed in Table 5.

TABLE 5
Exemplary oligonucleotide sequences.

| | | | | Coordinate in | Method IA | Method IB | Method IC | Method II |
|---|---|---|---|---|---|---|---|---|
| | | | | hg38 (chr strand | Example 2 | Example 4 | Example 6 | Example 8 |
| Line_ID | Name | Sequence and modification | Note | start end) | FIG. 6 | FIG. 7 | FIG. 8 | FIG. 9 |
| Oligo- | i5_N501_ | AATGATACGGCGACCACCGAGATCT | Common | | Yes | Yes | No | Yes |
| 01A-501 | UMI_Tn5-A | ACACTAGATCGCNNNNNNNNNNTC | | | | | | |
| | | GTCGGCAGCGTCAGATGTGTATAAG | | | | | | |
| | | AGACAG | | | | | | |
| | | (SEQ ID NO: 35) | | | | | | |
| Oligo- | i5_N502_ | AATGATACGGCGACCACCGAGATCT | Common | | Yes | Yes | No | Yes |
| 01A-502 | UMI_Tn5-A | ACACCTCTCTATNNNNNNNNNNTCG | | | | | | |
| | | TCGGCAGCGTCAGATGTGTATAAGA | | | | | | |
| | | GACAG (SEQ ID NO: 36) | | | | | | |
| Oligo- | i5_N503_ | AATGATACGGCGACCACCGAGATCT | Common | | Yes | Yes | No | Yes |
| 01A-503 | UMI_Tn5-A | ACACTATCCTCTNNNNNNNNNNTCG | | | | | | |
| | | TCGGCAGCGTCAGATGTGTATAAGA | | | | | | |
| | | GACAG (SEQ ID NO: 37) | | | | | | |
| Oligo- | i5_N504_ | AATGATACGGCGACCACCGAGATCT | Common | | Yes | Yes | No | Yes |
| 01A-504 | UMI_Tn5-A | ACACAGAGTAGANNNNNNNNNNTC | | | | | | |
| | | GTCGGCAGCGTCAGATGTGTATAAG | | | | | | |
| | | AGACAG (SEQ ID NO: 38) | | | | | | |
| Oligo- | i5_N505_ | AATGATACGGCGACCACCGAGATCT | Common | | Yes | Yes | No | Yes |
| 01A-505 | UMI_Tn5-A | ACACGTAAGGAGNNNNNNNNNNTC | | | | | | |
| | | GTCGGCAGCGTCAGATGTGTATAAG | | | | | | |
| | | AGACAG (SEQ ID NO: 39) | | | | | | |
| Oligo- | i5_N506_ | AATGATACGGCGACCACCGAGATCT | Common | | Yes | Yes | No | Yes |
| 01A-506 | UMI_Tn5-A | ACACACTGCATANNNNNNNNNNTC | | | | | | |
| | | GTCGGCAGCGTCAGATGTGTATAAG | | | | | | |
| | | AGACAG (SEQ ID NO: 40) | | | | | | |
| Oligo- | i5_N507_ | AATGATACGGCGACCACCGAGATCT | Common | | Yes | Yes | No | Yes |
| 01A-507 | UMI_Tn5-A | ACACAAGGAGTANNNNNNNNNNTC | | | | | | |
| | | GTCGGCAGCGTCAGATGTGTATAAG | | | | | | |
| | | AGACAG (SEQ ID NO: 41) | | | | | | |
| Oligo- | i5_N508_ | AATGATACGGCGACCACCGAGATCT | Common | | Yes | Yes | No | Yes |
| 01A-508 | UMI_Tn5-A | ACACCTAAGCCTNNNNNNNNNNTC | | | | | | |
| | | GTCGGCAGCGTCAGATGTGTATAAG | | | | | | |
| | | AGACAG (SEQ ID NO: 42) | | | | | | |
| Oligo-02A | i5_N501_ | AATGATACGGCGACCACCGAGATC/ | | | No | NO | Yes | NO |
| | UMI_Tn5-A | ideoxyU/ACACTAGATCGCNNNNNNN | | | | | | |
| | Uracil | NNN/ideoxyU/CGTCGGCAGCGTCAGA | | | | | | |
| | | TGTGTATAAGAGACAG | | | | | | |
| | | (SEQ ID NO: 43) | | | | | | |
| Oligo-03A | Tn5- | CTGTCTCTTATACACATCT/3ddC/ | Common | | Yes | Yes | Yes | Yes |
| | b_3'blocked | (SEQ ID NO: 44) | | | | | | |
| Oligo-04A | Primer | AATGATACGGCGACCACCGAGATCT | Common | | Yes | Yes | Yes | Yes |
| | 1/3/5 | ACAC (SEQ ID NO: 45) | | | | | | |
| Oligo-05A | Blocker 0- | +A+GG+GC+TC+CG+AT+GT+AT+AA | target | chr3 − | Yes | Yes | Yes | No |
| | CCR5#1_L | +TA+AT+T+G/3 AmMO/ | specific, for | 46372938 | | | | |
| | | (SEQ ID NO: 46) | CCR5#1 | 46372960 | | | | |
| | | | Left side | | | | | |
| | | | rearrangement | | | | | |
| Oligo-09A | Blocker 0- | +T+GG+GC+AA+CA+TG+CT+GG+TC | target | chr3 + | Yes | Yes | Yes | No |
| | CCR5#1_R | +AT+CC+T+C/3AmMO/ | specific. for | 46373041 | | | | |
| | | (SEQ ID NO: 47) | CCR5#1 | 46373062 | | | | |
| | | | Right side | | | | | |
| | | | rearrangement | | | | | |
| Oligo-13A- | Primer | CAAGCAGAAGACGGCATACGAGAT | common | | Yes | Yes | Yes | Yes |
| 701 | 6_N701 | AGCGGAATGTGACTGGAGTTCAGA | | | | | | |
| | | CGTGT (SEQ ID NO: 48) | | | | | | |
| Oligo-13A- | Primer | CAAGCAGAAGACGGCATACGAGAT | common | | Yes | Yes | Yes | Yes |
| 702 | 6 N702 | GATCATGCGTGACTGGAGTTCAGAC | | | | | | |
| | | GTGT (SEQ ID NO: 49) | | | | | | |
| Oligo-13A- | Primer | CAAGCAGAAGACGGCATACGAGAT | common | | Yes | Yes | Yes | Yes |
| 703 | 6_N703 | AAGACGGAGTGACTGGAGTTCAGA | | | | | | |
| | | CGTGT (SEQ ID NO: 50) | | | | | | |
| Oligo-13A- | Primer | CAAGCAGAAGACGGCATACGAGAT | common | | Yes | Yes | Yes | Yes |
| 704 | 6_N704 | CGAGTCCTGTGACTGGAGTTCAGAC | | | | | | |
| | | GTGT (SEQ ID NO: 51) | | | | | | |
| Oligo-13A- | Primer | CAAGCAGAAGACGGCATACGAGAT | common | | Yes | Yes | Yes | Yes |
| 705 | 6_N705 | TCCTCAGGGTGACTGGAGTTCAGAC | | | | | | |
| | | GTGT (SEQ ID NO: 52) | | | | | | |
| Oligo-13A- | Primer | CAAGCAGAAGACGGCATACGAGAT | common | | Yes | Yes | Yes | Yes |
| 706 | 6_N706 | GTACGGATGTGACTGGAGTTCAGAC | | | | | | |
| | | GTGT (SEQ ID NO: 53) | | | | | | |
| Oligo-13A- | Primer | CAAGCAGAAGACGGCATACGAGAT | common | | Yes | Yes | Yes | Yes |
| 707 | 6_N707 | CATCTCTCGTGACTGGAGTTCAGAC | | | | | | |
| | | GTGT (SEQ ID NO: 54) | | | | | | |
| Oligo-13A- | Primer | CAAGCAGAAGACGGCATACGAGAT | common | | Yes | Yes | Yes | Yes |
| 710 | 6_N710 | GTCGGAGCGTGACTGGAGTTCAGAC | | | | | | |
| | | GTGT (SEQ ID NO: 55) | | | | | | |
| Oligo-13A- | Primer | CAAGCAGAAGACGGCATACGAGAT | common | | Yes | Yes | Yes | Yes |
| 711 | 6_N711 | ACGGAGAAGTGACTGGAGTTCAGA | | | | | | |
| | | CGTGT (SEQ ID NO: 56) | | | | | | |
| Oligo-13A- | Primer | CAAGCAGAAGACGGCATACGAGAT | common | | Yes | Yes | Yes | Yes |
| 712 | 6_N712 | AGGAGATGGTGACTGGAGTTCAGA | | | | | | |
| | | CGTGT (SEQ ID NO: 57) | | | | | | |
| Oligo-13A- | Primer | CAAGCAGAAGACGGCATACGAGAT | common | | Yes | Yes | Yes | Yes |
| 714 | 6_N714 | AGTACTCGGTGACTGGAGTTCAGAC | | | | | | |
| | | GTGT (SEQ ID NO: 58) | | | | | | |
| Oligo-13A- | Primer | CAAGCAGAAGACGGCATACGAGAT | common | | Yes | Yes | Yes | Yes |
| 715 | 6_N715 | GGACTCTAGTGACTGGAGTTCAGAC | | | | | | |
| | | GTGT (SEQ ID NO: 59) | | | | | | |
| Oligo-60 | CCR5#1C | CTGATGTGTGGCAGTTTGGGAC | Validation | Chr13 − | Yes | Yes | Yes | Yes |
| | 13 | (SEQ ID NO: 60) | | 24886436 | | | | |
| | | | | 24886458 | | | | |

For site specific oligos, the “R” or “L” in the oligo name is defined by which side the blocker/probe 0 is annealed to relative to the cleavage site, when the genomic DNA is kept its +/− strands orientation, despite the target sequence (PAM) is on (+) or (−) strand.

Exemplary oligonucleotide sets used are shown in Table 6 and Table 7 below. For example, for CCR5 #1, a first reaction was performed to detect rearrangements on the left-side of the on-target cleavage site, and a second reaction was performed to detect rearrangements on the right-side of the on-target cleavage site. For each reaction, a set of target specific primers and blocker were used. For CCR5 #1 left side, Oligos 04A/05A/19/20 were used and for CCR5 #1 right side, Oligos 04A/09A/10A/23/24 were used.

TABLE 6
Target-specific primers and blocker

| | for CCR5#1 Left side | | for CCR5#1 Right | |
|---|---|---|---|---|
| | rearrangement | | side rearrangement | |
| Common | CCR5_L | | CCR5_R | |
| Name | Line ID | Name | Line_ID | Name |
| Blocker 0 | Oligo- | Blocker 0 - | Oligo- | Blocker 0 - |
| | 05A | CCR5#1 L | 09A | CCR5#1 R |
| Primer 2 | Oligo- | Primer 2 - | Oligo- | Primer 2 - |
| | 19 | CCR5#1 L | 23 | CCR5#1 R |
| Primer 4 | Oligo- | Primer 4 - | Oligo- | Primer 4 - |
| | 20 | CCR5#1 L | 24 | CCR5#1 R |

TABLE 7
Transposome oligonucleotides
UMI version

| Line_ID | Name |
|---|---|
| Oligo-01A-501 | i5_N501_UMI_Tn5-A |
| Oligo-01A-502 | i5_N502_UMI_Tn5-A |
| Oligo-03A | Tn5 transposome bottom |

Exemplary sgRNA target sequences used are shown in Table 8 below.

TABLE 8

| | Sequence | Coordinate in hg38 |
|---|---|---|
| Name | (PAM is underlined) | (chr strand start end) |
| CCR5#1 target | GTGAGTAGAGCGGAGGCAGGAGG | chr3-46372995 46373017 |
| sequence | (SEQ ID NO: 30) | |

Additional reagents included unloadedTn5 Transposase (Diagenode, C01070010, Diagenode), 2× Tagmentation buffer (Diagenode, C01019043), Platinum™ SuperFi II PCR Master Mix (ThermoFisher, Cat #12368010), dNTP Mix (10 mM each) (ThermoFisher, Cat #R0194), DNA purification kit:DNA Clean & Concentrator-5 (Zymo Research, Cat #D4004), AMPure XP Reagent (Beckman Coulter, Cat #A63880), Glycerol (MilliporeSigma, 356350500ML), 10% SDS (Fisher, BP2436200), 5M NaCl (Invitrogen, AM9760G), Tris HCl 1M, pH8.0 (Invitrogen, 15568-025), Genomic DNA (extracted from edited and unedited 293T cells), NEBNext library Quant Kit for Illumina (NEB, E7630), Illumina NextSeq 1000/2000 P1 Reagents (300 Cycles) (Illumina, Cat #20050264), PhiX Control v3 (Illumina, Cat #FC-110-3001).

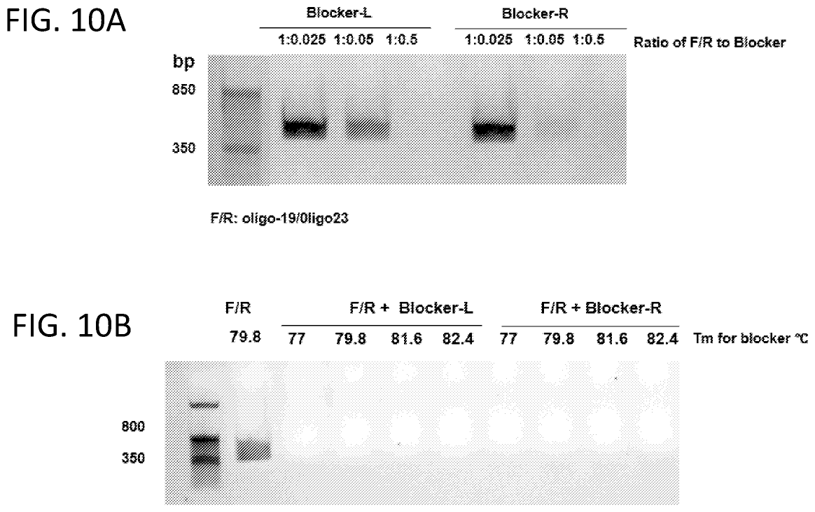

Before library preparation, we tested the ability of the LNA blocker to efficiently block the amplification of unintended targets, including wild type and edits with small indels. We included LNA blocker (Oligo05-A and Oligo09-A) in the PCR to amplify the amplicon using wild type genomic DNA. The PCR mixture was prepared as follows: 50 ng gDNA, 25 μL 2× PCR Master Mix, 2.5 μL Primer F (Oligo-19, 10 μM), 2.5 μL Primer R (Oligo-23, 10 μM), different concentration of Blocker, and add ddH2O up to 50 μL. PCR was performed using the following program: initial denaturing at 98° C. for 30 seconds, 35 cycles of the 4-step denaturing-annealing-amplification (10 seconds at 98° C., 10 seconds at 80° C., 10 second at 60° C., seconds at 72° C.), and final extension at 72° C. for 5 minutes, and hold at 4° C. PCR products were directly profiled in 2% EX agarose gel. As shown in FIG. 10A, when the concentration of Blockers reaches 50% of the PCR primers, both of which can efficiently block the amplification of the amplicon. To further optimize the blocking effect of the Blocker oligo, different temperatures were tested as shown in FIG. 10B. The temperatures tested, from 77° C. to 82.4° C., all achieved complete elimination of PCR products. 78° C. was used for 4-step denaturing-annealing-amplification (10 seconds at 98° C., 10 seconds at 78° C., 10 second at 60° C., seconds at 72° C.) for library preparation.

In the first step, transposon-end containing oligos (e.g., Oligo-01A-501 and Oligo-03A, or Oligo-01A-502 and Oligo-03A) were resuspend in Annealing Buffer (40 mM Tris-HCl (pH8.0), 50 mM NaCl) to stock concentration of 100 μM. In a PCR tube, 10 μl oligo-01, 10 μl oligo-03 were mixed, vortexed and placed in PCR tubes in a thermocycler with the following program: 5 minutes at 95° C., cool to 65° C. (−0.1° C./second), 5 minutes at 65° C., cool to 25° C. (−0.1° C./second), 5 minutes at 25° C., and hold at 4° C.

Transposome complexes were assembled following the procedure below, as recommended by the manufacturer. The reagents were mixed in a PCR tube: 10 μL Tn5 transposase (2 ug/ul), 10 μL annealed adaptor. The reagents were mixed thoroughly and incubated at 23° C. for 30 minutes. 10 μl glycerol was added and mixed. The assembled transposome complex was stored at −20° C.

The workflow for this example is shown in FIG. 6. The tagmentation reaction was performed in which genomic DNA extracted from gene edited samples or controls (using standard methods) was tagmented with the assembled transposome complex using the following procedure. The reaction mixture contained: 100 ng genomic DNA from unedited control or edited sample, 20 μL 2× Tagmentation buffer, 200 ng loaded Tn5 Transposase, and add H2O up to 40 μL. The reaction mixture was mixed thoroughly and incubated at 55° C. for 15 minutes. 10 μL 0.2% SDS was added. And then Tn5 was inactivated for 10 min at 70° C. The tagmented DNA was purified using a Zymo column following the manufacturer's instructions and eluted in 21 μL.

Tagmented DNA from the previous step was used for two separate PCR reactions for each target, one reaction to detect genome rearrangements on the left-side of the target and the other reaction to detect genome rearrangements on the right-side of the target. Each PCR was then performed with a common primer annealed to the Tn5 adapter region, a target specific blocker and a target specific primer. Primer 1 (Oligo-04A, common to all targets and rearrangement of both sides), Primer 2 (e.g., Oligo-19 for CCR5 #1 left-side rearrangement, or Oligo-23 for CCR5 #1 right-side rearrangement), and Blocker 0 (e.g., Oligo-05A for CCR5 #1 left-side rearrangement, or Oligo-09A for CCR5 #1 right-side rearrangement) using SuperFi II Polymerase PCR Master Mix (ThermoFisher, Cat #12368010). PCR mixture was prepared as follows: 25 μL 2× PCR Master Mix, 10 μL Tagmented DNA, and 1.25 μL Primer 1 (10 μM), 2.5 μL Primer 2 (10 μM), 10 μL Blocker 0 (10 μM), and add H2O up to 50 μL. PCR was performed using the following program: initial denaturing at 98° C. for 30 seconds, 15 cycles of the 4-step denaturing-annealing-amplification (10 seconds at 98° C., 10 seconds at 78° C., 10 second at 60° C., 60 seconds at 72° C.), and final extension at 72° C. for 5 minutes, and hold at 4° C. PCR products were purified using AMpure XP beads (1×) and eluted in 12 μL.

Nested PCR was then performed with the PCR product from the previous step, Primer 3 (Oligo-04A, common to all targets and rearrangement of both sides), Primer 4 (e.g., Oligo-20 for CCR5 #1 left-side rearrangement, or Oligo-24 for CCR5 #1 right-side rearrangement), and Blocker 0 (e.g., Oligo-05A for CCR5 #1 left-side rearrangement, or Oligo-09A for CCR5 #1 right-side rearrangement) using SuperFi II Polymerase PCR Master Mix (ThermoFisher, Cat #12368010). PCR mixture was prepared as follows: 25 μL 2× PCR Master Mix, 10 μL PCR product, 1.25 μL Primer 3 (10 μM), 2.5 μL Primer 4 (10 μM), 10 μL Blocker 0 (10 μM), and add H2O up to 50 μL. PCR was performed using the following program: initial denaturing at 98° C. for 30 seconds, 15 cycles of the 4-step denaturing-annealing-amplification (10 seconds at 98° C., 10 seconds at 78° C., 10 second at 60° C., seconds at 72° C.), and final extension at 72° C. for 5 minutes, and hold at 4° C. PCR products were purified using AMpure XP beads, and eluted in 12 μL.

Tag PCR was then performed with sequencing primers. PCR was performed with PCR products from the previous step, Primer 5 (Oligo-04A, common to all targets and rearrangement of both sides), Primer 6 (e.g., any Oligo-13A, or Illumina indexing primer from New England Biolabs, NEB #E7611A), and Blocker 0 (e.g., Oligo-05A for CCR5 #1 left-side rearrangement, or Oligo-09A for CCR5 #1 right-side rearrangement) using SuperFi II Polymerase PCR Master Mix (ThermoFisher, Cat #12368010). PCR mixture was prepared as follows: 25 μL 2× PCR Master Mix, 10 μL PCR product, 5 μL Primer 5 (10 μM), 5 μL Primer 6 (10 μM), 5 μL Blocker 0 (20 μM). PCR was performed using the following program: initial denaturing at 98° C. for 30 seconds, 15 cycles of the 4-step denaturing-annealing-amplification (10 seconds at 98° C., 10 seconds at 78° C., 10 second at 60° C., seconds at 72° C.), and final extension at 72° C. for 5 minutes, and hold at 4° C. PCR products were purified using AMpure XP beads, and eluted in 52 μL.

50 μL of the PCR product was transferred to a new tube for library quantification using NEBNext library Quant Kit for Illumina (NEB, E7630) following the manufacture's instructions. Libraries were normalized and pooled for sequencing.

Next generation sequencing was then performed using the PCR product from the previous step, and sequencing was performed using an Illumina NextSeq 2000 with 20% PhiX (Illumina, Cat #FC-110-3001).

Paired-end sequencing reads from Illumina sequencing were processed using a customized NGS analysis pipeline. The sequence reads were first checked for overall quality using FastQC followed by consolidating FastQC results using MultiQC. The reads were then subjected to Adapter and primer sequence trimming using Cutadapt (DOI:10.14806/ej.17.1.200) followed by trimming low quality bases using Trimmomatic (Bolger et al, Bioinformatics. 2014; 30 (15): 2114-20). The trimming in the reads was verified by running FastQC and MultQC steps on trimmed reads fastq files to ascertain the removal of adaptors. The resulting reads were aligned with human genome reference sequence (hg38) using BWA-MEM (Li and Durbin, Bioinformatics, 2009; 25:1754-1760) tool with default parameters. The generated Sequence Alignment Map (SAM) file was sorted by coordinate and converted to Binary Alignment Map (BAM) file using PicardTools (http://broadinstitute.github.io/picard). The BAM file was then filtered to remove low-quality reads and to keep the reads with quality score >30 using Samtools. The resulting high quality BAM file was then checked using the CollectInsertSizeMatrix and CollectAlignemntSummary Matrics modules from PicardTools followed by FastQC and MultiQC report generation. The reads in the sorted high quality read BAM were grouped based on UMIs using UMI-Tools with paired option and saved as grouped BAM followed by BAM indexing using Samtools. The grouped BAM file was then de-duplicated using UMI-Tools with paired option to remove PCR duplicate reads. The FGSV tool was then used to discover the structural variation pileup by searching for split read mapping and read pairs that map across breakpoints in the BAM file using the FGSV SVPileup module. The AggregateSvPileup module of FGSV then aggregated information across nearby pileups to call structural variants. Only the pileups containing at least one breakpoint on the target were considered as real hits, and as high confidence hits with at least 10 split reads. IGV (Thorvaldsdóttir et al, Brief Bioinform. 2013; 14:178-192) was used to facilitate the identification and analysis of rearrangements at genomic scale.

To validate the DNA rearrangements identified by experiment above, PCR was performed with genomic DNA after gene editing, Primer for inter-chromosomal rearrangement event CCR5 #1C13 (Oligo-60, common to rearrangement of both sides), Primer for CCR5-side (e.g., Oligo-20 for CCR5 #1 left-side rearrangement, or Oligo-23 for CCR5 #1 right-side rearrangement), and Blocker 0 (e.g., Oligo-05A for CCR5 #1 left-side rearrangement, or Oligo-09A for CCR5 #1 right-side rearrangement) using SuperFi II Polymerase PCR Master Mix (ThermoFisher, Cat #12368010). The PCR mixture was prepared as follows: 100 ng gDNA, 25 μL 2× PCR Master Mix, 5 μL Primer CCR5 #1C13 (10 μM), 5 μL Primer CCR5-side (10 μM), 5 μL Blocker 0 (20 μM). PCR was performed using the following program: initial denaturing at 98° C. for 30 seconds, 35 cycles of the 4-step denaturing-annealing-amplification (10 seconds at 98° C., 10 seconds at 78° C., 10 second at 60° C., 60 seconds at 72° C.), and final extension at 72° C. for 5 minutes, and hold at 4° C. PCR products were directly profiled in 2% EX agarose gel. Since the genomic DNA for all the experiments performed in this application is from the same batch, therefore, the PCR method is the same for all methods and the results are applicable to all method as well.

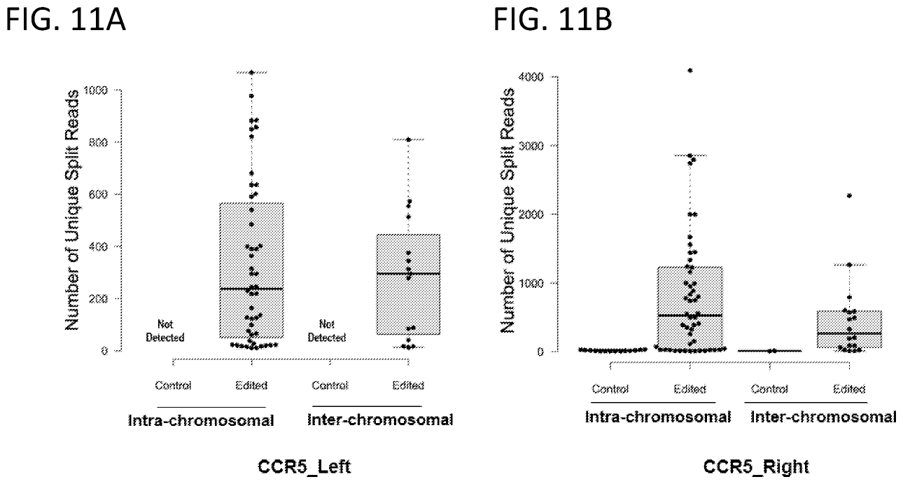

Summary of DNA rearrangement events detected for Example 2 in control and edited cells in Table 9 below. To remove the noise introduced by PCR in library preparation for next generation sequencing, we set a threshold at 10 split reads for high confident hits and one breakpoint must be located at the on-target site. In control cells, 0 hits passed the filters for high confidence for the left-side events, 18 hits were enriched for the right-side events in Table 9. However, the number of the split reads that supported these hits was very low compared to events in edited cells in FIGS. 11A-B. In edited samples, in total, 63 and 68 hits were captured for the left side and right-side events, respectively. These data support that the hits captured in edited cells are produced or increased by CRISPR/Cas9 gene editing.

TABLE 9
Summary of DNA rearrangement events detected for Example 2

| Samples | Side | Intra Chromosomal | Inter Chromosomal |
|---|---|---|---|
| Control | Left | 0 | 0 |
| CCR5 edited | | 48 | 15 |
| Control | Right | 14 | 4 |
| CCR5 edited | | 50 | 18 |

Note:
DNA rearrangment events with split read number <10 were filtered out. One breakpoint must be located at the on-target cutting site +/−25 bp.

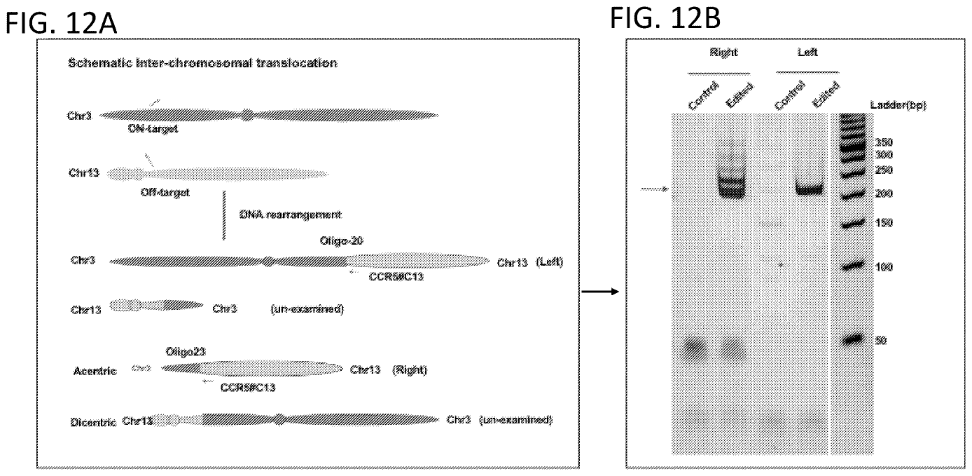

DNA rearrangements identified by Example 2 in CCR5 edited cells are displayed in Table 10 below. The genomic coordinates of the DNA breakpoints between DNA rearrangements, number of unique split-reads based on UMI-tools, extracted reads to show the split reads, and note to further explain the potential mechanism for this DNA rearrangements were included in the table. It is worth noting that the DNA rearrangements between CCR5 and its homologous gene CCR2, inter-chromosomal translocation between CCR5 on chromosome 3 and RNF17/CENPJ on chromosome 13 were also identified by CAST-seq (Turchiano et al, 2021). The presence of fused DNA of chr3/chr13 and acentric chromosome were also validated using PCR (FIGS. 12A-B). Therefore, SAFER detection Method I_{A} can efficiently capture intra- and inter-chromosomal rearrangements.

TABLE 10
Examples of split reads for DNA rearrangement events.

| | | | | Unique | | |
|---|---|---|---|---|---|---|
| | | On-target | Non-target | Split_ | | |
| Catergory | Side | breakpoint | breakpoint | reads | Exemplar read | Note |
| Intra- | Left | chr3: | chr3: | 559 | Read name: | This event represents |
| chromosomal | | 46373018- | 46357590- | | VH01033: 30: | an intergenic inversion |
| rearrangement | | 46373029 | 46357600 | | AAF5TJWM5: 1: | that is caused by off- |
| | | | | | 1202: 15618: | target editing or |
| | | | | | 35494: rCTA | homology mediated |
| | | | | | CCGATCA | recombination. |
| | | | | | Sequence: | Predicted off-target |
| | | | | | GCTCTTCAGCC | site: |
| | | | | | TTTTGCAGTTTA | CACCAGCGAGTAG |
| | | | | | TCAGGATGAGG | AGCGGAGGCAGGA |
| | | | | | ATGACCAGCAT | G (SEQ ID NO: 61) |
| | | | | | GTTGCCCACAA | chr3:46357657- |
| | | | | | AACCAAAGATG | 46357683 and |
| | | | | | AACACCTTTTTT | homology sequence in |
| | | | | | GATTATGATTA | CCR2 and CCR5: |
| | | | | | CGGTGCTCCCT | CTGGTGTTCATCTT |
| | | | | | GTCATAAATTT | TGGTTTTGTGGGCA |
| | | | | | GACGTGAAGCA | ACATGCTGGTC |
| | | | | | AATTGGGGCCC | (SEQ ID NO: 62) |
| | | | | | AACTCCTGCCT | Gene: CCR2, CCR5 |
| | | | | | CCGCTC (SEQ ID | |
| | | | | | NO: 64) | |
| | | | | | Alignments: | |
| | | | | | chr3: | |
| | | | | | 46,373,018- | |
| | | | | | 46,373,091 (+) | |
| | | | | | chr3: | |
| | | | | | 46,357,593- | |
| | | | | | 46,357,671 (−) | |
| | | | | | underlined | |
| | Left | chr3: | chr3: | 2643 | Read name: | The event represents a |
| | | 46373000- | 46372658- | | VH01033: 30: | large deletion caused |
| | | 46373015 | 46372689 | | AAF5TJWM5: 1: | by CRISPR/Cas9 editing. |
| | | | | | 2404: 56614: | No off-target site was |
| | | | | | 2950: rTCTA | predicted at the non- |
| | | | | | CCGGAA | target breakpoint site. |
| | | | | | Sequence: | Gene: CCR5 |
| | | | | | GTGCTATAGAG | |
| | | | | | CACAAGATTTT | |
| | | | | | ATTTGGTGAGA | |
| | | | | | TGGTGCTCTAC | |
| | | | | | TCACTGGTGTT | |
| | | | | | CATCTTTGGTTT | |
| | | | | | TGTGGGCAACA | |
| | | | | | TGCTGGTCATC | |
| | | | | | CTCATCCTGAT | |
| | | | | | AAACTGCAAAA | |
| | | | | | GGCTGAAGAGC | |
| | | | | | (SEQ ID NO: 65) | |
| | | | | | Alignments: | |
| | | | | | chr3: | |
| | | | | | 46,372,619- | |
| | | | | | 46,372,658 (+) | |
| | | | | | underlined | |
| | | | | | chr3: | |
| | | | | | 46,373,007- | |
| | | | | | 46,373,092 (+) | |
| | Left | chr3: | chr3: | 1284 | Read name: | The event represents a |
| | | 46373000- | 46374369- | | VH01033: 30: | large deletion caused |
| | | 46373015 | 46374387 | | AAF5TJWM5: 1: | by CRISPR/Cas9 editing. |
| | | | | | 1102: 51274: | This intragenic |
| | | | | | 48462: rTTA | Inversion may be |
| | | | | | CCTATCA | occurred between on- |
| | | | | | Sequence: | target CRISPR editing |
| | | | | | CCCCATAGCAA | and naturally happened |
| | | | | | GACAAAGACCT | breakpoint. |
| | | | | | GTTTTAAACAC | No off-target site was |
| | | | | | ATTTACAACCT | predicted at the non- |
| | | | | | ATATGTCTCCG | target breakpoint site. |
| | | | | | CTCTACTCACT | Gene: CCR5 |
| | | | | | GGTGTTCATCTT | |
| | | | | | TGGTTTTGTGG | |
| | | | | | GCAACATGCTG | |
| | | | | | GTCATCCTCAT | |
| | | | | | CCTGATAAACT | |
| | | | | | GCAAAAGGCTG | |
| | | | | | AAGAGC (SEQ | |
| | | | | | ID NO: 66) | |
| | | | | | Alignments: | |
| | | | | | chr3: | |
| | | | | | 46,373,002- | |
| | | | | | 46,373,091 (+) | |
| | | | | | chr3: | |
| | | | | | 46.374,369- | |
| | | | | | 46,374,419 (-) | |
| | | | | | underlined | |
| | Right | chr3: | chr3: | 28 | Read name: | This event represents |
| | | 46373000- | 46357607- | | VH01033:30:AAF | an intergenic |
| | | 46373000 | 46357607 | | 5TJWM5:1:1404:8 | inversion that is |
| | | | | | 328:41230:rCAGC | caused by off-target |
| | | | | | ACCATG | editing or homology |
| | | | | | Sequence: | mediated |
| | | | | | TACATCGGAGC | recombination. |
| | | | | | CCTGCCAAAAA | Predicted off-target |
| | | | | | ATCAATGTGAA | site: |
| | | | | | GCAAATCGCAG | CACCAGCGAGTAG |
| | | | | | CCCGCCTCCTC | AGCGGAGGCAGGA |
| | | | | | ATAATCAAAAA | G (SEQ ID NO: 61) |
| | | | | | AGGTGGTGACT | chr3: 46357657- |
| | | | | | TCTTCACCGCTC | 46357683 and |
| | | | | | TCGTTGGTATTT | homology sequence in |
| | | | | | CTGGTGTTCGTT | CCR2 and CCR5: |
| | | | | | (SEQ ID NO: 67) | CTGGTGTTCATCTT |
| | | | | | Alignments: | TGGTTTTGTGGGCA |
| | | | | | chr3: | ACATGCTGGTC |
| | | | | | 46,372,947- | (SEQ ID NO: 62) |
| | | | | | 46,373,000 (+) | Gene: CCR2, CCR5 |
| | | | | | chr3: | |
| | | | | | 46,357,556- | |
| | | | | | 46,357,606 (−) | |
| | | | | | underlined | |
| | Right | chr3: | chr3: | 391 | Read name: | This event represents |
| | | 46372992- | 46357616- | | VH01033: 30: | an intergenic inversion |
| | | 46373001 | 46357619 | | AAF5TJWM5: 1: | that is caused by off- |
| | | | | | 1301: 13725: | target editing |
| | | | | | 35115: rGTC | or homology |
| | | | | | GACCCAA | mediated |
| | | | | | Sequence: | recombination. |
| | | | | | TACATCGGAGC | Predicted off-target |
| | | | | | CCTGCCAAAAA | site: |
| | | | | | ATCAATGTGAA | CACCAGCGAGTAG |
| | | | | | GCAAATCGCAG | AGCGGAGGCAGGA |
| | | | | | CCCGCCTCCCT | G (SEQ ID NO: 61) |
| | | | | | GTCATAAATTT | chr3:46357657- |
| | | | | | GACGTGAAGCA | 46357683 and |
| | | | | | AATTGGGGCCC | homology sequence in |
| | | | | | AACTCCTGCC | CCR2 and CCR5: |
| | | | | | (SEQ ID NO: 68) | CTGGTGTTCATCTT |
| | | | | | Alignments: | TGGTTTTGTGGGCA |
| | | | | | chr3: | ACATGCTGGTC |
| | | | | | 46,372,947- | (SEQ ID NO: 62) |
| | | | | | 46,372,999 (+) | Gene: CCR2, CCR5 |
| | | | | | chr3: | |
| | | | | | 46,357,616- | |
| | | | | | 46,357,664 (+) | |
| | | | | | underlined | |
| | Right | chr3: | chr3: | 4074 | Read name: | The event represents a |
| | | 46372986- | 46373246- | | VH01033: 30: | large deletion caused |
| | | 46373002 | 46373288 | | AAF5TJWM5: 1: | by CRISPR/Cas9 editing. |
| | | | | | 1303: 31164: | No off-target site was |
| | | | | | 20443: rAGA | predicted at the non- |
| | | | | | TCCGGTT | target breakpoint site. |
| | | | | | Sequence: | Gene: CCR5 |
| | | | | | TACATCGGAGC | |
| | | | | | CCTGCCAAAAT | |
| | | | | | CAATGTGAAGC | |
| | | | | | AAATCGCAGCC | |
| | | | | | CGCCTCCTGAC | |
| | | | | | AATCGATAGGT | |
| | | | | | ACCTGGCTGTC | |
| | | | | | GTCCATGCTGT | |
| | | | | | GTTTGCTTTAA | |
| | | | | | AAGCCAGGACG | |
| | | | | | GTCACCTTTGG | |
| | | | | | GGTGGTGACAA | |
| | | | | | GTGTGATCACT | |
| | | | | | TGGGTGG (SEQ | |
| | | | | | ID NO: 69) | |
| | | | | | Alignments: | |
| | | | | | chr3: | |
| | | | | | 46,372,946- | |
| | | | | | 46,372,995 (+) | |
| | | | | | chr3: | |
| | | | | | 46,373,262- | |
| | | | | | 46,373,365 (+) | |
| | | | | | underlined | |
| | Right | chr3: | chr3: | 1686 | Read_name | The event represents a |
| | | 46372984- | 46368151- | | VH01033: 30: | large deletion caused |
| | | 46373001 | 46368163 | | AAF5TJWM5: 1: | by CRISPR/Cas9 editing. |
| | | | | | 2104: 51122: | This intragenic |
| | | | | | 6774: rCCGG | Inversion may be |
| | | | | | GTGTCT | occurred between on- |
| | | | | | Sequence: | target CRISPR editing |
| | | | | | CTCCCCATCCT | and naturally happened |
| | | | | | GTTGACGATGC | breakpoint. |
| | | | | | TCTGAAAATAT | No off-target site was |
| | | | | | GGTCCAGAAAT | predicted at the non- |
| | | | | | GGTGTGAGCAA | target breakpoint site. |
| | | | | | GGAGACAGCAA | Gene: CCR5 |
| | | | | | AGCAATGCTTG | |
| | | | | | GAACATAGGTG | |
| | | | | | CAGTGACTAGA | |
| | | | | | CATGAGGCGGG | |
| | | | | | CTGCGATATGA | |
| | | | | | TTCACATTGATT | |
| | | | | | TTTTGGCAGGG | |
| | | | | | CTCCGAT (SEQ | |
| | | | | | ID NO: 70) | |
| | | | | | Alignments: | |
| | | | | | chr3: | |
| | | | | | 46,368,056- | |
| | | | | | 46,368,162 (+) | |
| | | | | | chr3: | |
| | | | | | 46,372,946- | |
| | | | | | 46,372,997 (−) | |
| Inter | Left | chr3: | chr13: | 279 | Read name: | These events represents |
| chromosomal | | 46373002- | 24886337- | | VH01033: 21: | an interchromosomal |
| rearrangement | | 46373008 | 24886341 | | AACCM73M5: 1: | translocation that is |
| | | | | | 2403: 50308: | mediated by Off-target |
| | | | | | 18720: rACT | editing |
| | | | | | AGCCAAG | On-target site: |
| | | | | | Sequence: | chr3:46372995- |
| | | | | | GATGAGGATGA | 46373017 (−) |
| | | | | | CCAGCATGTTG | GTGAGTAGAGCGG |
| | | | | | CCCACAAAACC | AGGCAGGAGG (SEQ |
| | | | | | AAAGATGAACA | ID NO: 30) |
| | | | | | CCAGTGAGTAG | OFF-target site: |
| | | | | | AGCGGGTGGTC | G()GAGGAGAGCGG |
| | | | | | AGCCAACAAGC | AGGCAGGTGG |
| | | | | | TGCTGGGCGTG | Chr13: 24886320- |
| | | | | | TGAGGCGGCTG | 24886342 (+) |
| | | | | | TGCTGTGCCTG | Gene: CCR5 and |
| | | | | | TGAGACAGGAA | RNF17/CENPJ |
| | | | | | TGGTTCCATTA | |
| | | | | | CAC (SEQ ID NO: | |
| | | | | | 71) | |
| | | | | | Alignments: | |
| | | | | | chr3: | |
| | | | | | 46373004- | |
| | | | | | 46373063 (-) | |
| | | | | | chr13: | |
| | | | | | 248863338- | |
| | | | | | 248863413 (+) | |
| | | | | | underlined | |
| | Right | chr3: | chr13: | 464 | Read name: | |
| | | 46372994- | 24886335- | | VH01033: 30: | |
| | | 46373001 | 24886341 | | AAF5TJWM5: | |
| | | | | | 1: 2402: | |
| | | | | | 21375: 40681: | |
| | | | | | rATC | |
| | | | | | CCGGCTC | |
| | | | | | Sequence: | |
| | | | | | TACATCGGAGC | |
| | | | | | CCTGCCAAAAA | |
| | | | | | ATCAATGTGAA | |
| | | | | | GCAAATCGCAG | |
| | | | | | CCCGCAGGTGG | |
| | | | | | TCAGCCAACAA | |
| | | | | | GCTGCTGGGCG | |
| | | | | | TGTGAGGCGGC | |
| | | | | | TGTGCTGTGCC | |
| | | | | | TGTGAGACAGG | |
| | | | | | AATGGTTCCAT | |
| | | | | | TACAC (SEQ ID | |
| | | | | | NO: 72) | |
| | | | | | Alignments: | |
| | | | | | chr3: 46372947- | |
| | | | | | 46372995 (+) | |
| | | | | | chr13: | |
| | | | | | 248863336- | |
| | | | | | 248863412 (+) | |
| | | | | | underlined | |

### Example 3: Exemplary Method for Method I_{B}

Exemplary oligonucleotides are listed in Table 1.

Additional reagents include Tn5 transposase, e.g., Robust Tn5 Transposase (Cat #EMQZ1422, Creative Biogene), DNA polymerase PCR master mix, e.g., Platinum™ SuperFi II PCR Master Mix (ThermoFisher, Cat #12368010), DNA polymerase with PCR buffer, e.g., Platinum™ SuperFi II DNA Polymerase (ThermoFisher, Cat #12361010), dNTP, e.g., dNTP Mix (10 mM each) (ThermoFisher, Cat #R0194), ddNTP, e.g., Dideoxynucleoside Triphosphate Set (MilliporeSigma, Cat #03732738001), DNA purification kit: Column kit, e.g., DNA Clean & Concentrator-5 (Zymo Research, Cat #D4004), Magnetic beads, e.g., AMPure XP Reagent (Beckman, Cat #A63880), Illumina sequencing kit, e.g., NextSeq 1000/2000 P1 Reagents (300 Cycles) (Cat #20050264), MiSeq Reagent Kit v3 (150-cycle) (Cat #MS-102-3001), MiSeq Reagent Kit v2 (300-cycles) (Cat #MS-102-2002), PhiX Control v3 (Illumina, Cat #FC-110-3001), STE buffer (10 mM Tris-HCl (pH 8.0), 1 mM EDTA, 0.1M NaCl).

In the first step, Tn5 transposomes are assembled. Transposon-end containing oligos (e.g., Oligo-01 and Oligo-02 for non-UMI version, or Oligo-26 and Oligo-02 for UMI version) are annealed in STE buffer with the following program: 1-10 minutes at 90-99° C., 40-500 cycles of 1 minutes at 90-99° C. (decrease temperature 0.2-2° C. every cycle until reaching 1-12° C.). Transposome complexes are assembled in TPS buffer following the procedure below, as recommended by the manufacturer (Creative Biogene, Cat #EMQZ1422). The reagents are mixed thoroughly and incubated at 15-40° C. for 5-120 minutes.

| Component | Volume | |
|---|---|---|
| Tn5 transposase | 1-10 | μL |
| Adaptor | 0.5-8 | μL |
| 10x TPS buffer | 2 | μL |

| | |
|---|---|
| Sterile water | add to 20 μL |

The workflow for this example is shown in FIG. 2. The tagmentation reaction is performed in which genomic DNA extracted from gene edited samples or controls (using standard methods) is tagmented with the assembled transposome complex following procedure:

| Component | Volume | |
|---|---|---|
| Genomic DNA | varies (5-500 ng) | |
| 5x LM buffer | 6 | μL |
| Transposome | 0.1-8 | μL |

| | |
|---|---|
| Sterile water | add to 30 μL |

The reaction components are mixed thoroughly and incubated at 30-65° C. for 5-120 minutes. Tagmented DNA is purified using column-based purification.

The 3′ ends of DNA molecules are then blocked. 3′ ends of DNA molecules are blocked using ddNTP and DNA polymerase SuperFi II (ThermoFisher, Cat #12361010). Product is purified using column-based purification.

Tagmented DNA from the previous step is used for two separate PCR reactions for each target, one reaction to detect genome rearrangement on the left-side of the target and the other reaction to detect genome rearrangement on the right-side of the target. Each PCR is then performed with a common primer annealed to the adapter region, a target specific blocker and a target specific primer. PCR is performed using 3-step PCR cycling protocol with tagmented DNA, Primer 1 (Oligo-04, common to all targets and rearrangement of both sides), Primer 2 (e.g., Oligo-05 for EMX1 left-side rearrangement, Oligo-15 for EMX1 right-side rearrangement, Oligo-19 for CCR5 #1 left-side rearrangement, or Oligo-23 for CCR5 #1 right-side rearrangement), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: initial denaturing at 90-99° C. for 1-5 minute, 20-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Nested PCR is then performed with the barcode tag. PCR is performed with PCR product from the previous step, Primer 3 (Oligo-06, common to all targets and rearrangement of both sides), Primer 4 (e.g., Oligo-07 for EMX1 left-side rearrangement, Oligo-16 for EMX1 right-side rearrangement, Oligo-20 for CCR5 #1 left-side rearrangement, or Oligo-24 for CCR5 #1 right-side rearrangement), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: initial denaturing at 90-99° C. for 1-5 minute, 4-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Tag PCR is then performed with sequencing primers. PCR is performed with PCR product from the previous step, Primer 5 (e.g., Oligo-08, an example of Illumina indexing primer from New England Bio Labs, NEB #E7603A), Primer 6 (e.g., Oligo-09, an example of Illumina indexing primer from New England Bio Labs, NEB #E7611A), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: initial denaturing at 90-99° C. for 1-5 minute, 4-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Next generation sequencing is then performed using the PCR product from the previous step, and sequencing is performed using an Illumina platform (e.g., Illumina MiSeq or NextSeq 2000) with optional >=0-95% PhiX (Illumina, Cat #FC-110-3001).

Paired-end sequencing reads from Illumina sequencing are merged using a paired-end reads merging software, e.g., PEAR (Zhang et al, Bioinformatics. 2014; 30:614-620), FLASH (Magoč and Salzberg, Bioinformatics. 2011; 27:2957-2963), BBMerge (Bushnell et al, PLoS One. 2017; 12:e0185056), or NGmerge (Gaspar, Bioinformatics. 2018; 19:536). The merged reads are then trimmed and filtered using software such as BBmap (https://jgi.doe.gov/data-and-tools/software-tools/bbtools/), samtools (Danecek et al, Gigascience. 2021; 10. doi: 10.1093/gigascience/giab008), or custom scripts to remove Illumina adaptor sequences, low quality reads, reads containing the target flanking sequence on the side of the target site where the blocking oligonucleotide or cleavage reagent binds, and reads that do not contain the target flanking sequence on the side of the target site where the target-specific primer hybridizes. Selected reads are aligned to a human reference genome, e.g., GRCh37 (hg19), GRCh38 (hg38), or Telomere-to-Telomere assembly (e.g., T2T-CHM13v2.0), using an alignment software, e.g., Bowtie 2 (Langmead and Salzberg, Nat Methods. 2012; 9:357-359), BWA (Li and Durbin, Bioinformatics. 2009; 25:1754-1760), or Minimap2 (Li, Bioinformatics. 2018; 34:3094-3100). The aligned BAM file is converted into bed file using BEDTools (Quinlan, Bioinformatics. 2014; 47:11.12.1-34). For methods using UMIs, UMIs are collapsed to remove redundant sequencing reads using software such as Gencore (Chen et al, Bioinformatics. 2019; 20:606), UMI-tools (Smith et al, Genome Res. 2017; 27:491-499), UMI-Reducer (github.com/smangul1/UMI-Reducer), or custom scripts.

Reads with candidate translocation break points within a suitable window flanking the target site (e.g., within 1, 3, 5, 10, 20, 40, 60, 80, 100, 200, or more bases) are identified and counted to quantify the number of rearrangements between the target site and other genomic loci. Statistical tests are then applied to establish a confidence score to each group of rearrangements at each distal rearranged locus (e.g., within a defined window of up 50, 100, 200, 500, 1000, 2000, 3000, or more bases at the distal rearranged locus). Visualization tools such as IGV (Thorvaldsdóttir et al, Brief Bioinform. 2013; 14:178-192) or Circos (Krzywinski et al, Genome Res. 2009; 19:1639-1645) are used to facilitate the identification and analysis of rearrangements at genomic scale.

### Example 4: Exemplary Method for Method I^{B}

Exemplary oligonucleotides used are listed in Table 5.

Additional reagents included unloadedTn5 Transposase (Diagenode, C01070010, Diagenode), 2× Tagmentation buffer (Diagenode, C01019043), Platinum™ SuperFi II PCR Master Mix (ThermoFisher, Cat #12368010), dNTP Mix (10 mM each) (ThermoFisher, Cat #R0194), DNA purification kit: DNA Clean & Concentrator-5 (Zymo Research, Cat #D4004), AMPure XP Reagent (Beckman Coulter, Cat #A63880), Glycerol (MilliporeSigma, 356350500ML), 10% SDS (Fisher, BP2436200), 5M NaCl (Invitrogen, AM9760G), Tris HCl 1M, pH8.0 (Invitrogen, 15568-025), Genomic DNA (extracted from edited and unedited 293T cells), ddNTP, Dideoxynucleoside Triphosphate Set (MilliporeSigma, Cat #03732738001), NEBNext library Quant Kit for Illumina (NEB, E7630), Terminal transferase (TdT, NEB, M0315S), USER, (NEB #M5508), Illumina NextSeq 1000/2000 P1 Reagents (300 Cycles) (Illumina, Cat #20050264), PhiX Control v3 (Illumina, Cat #FC-110-3001).

In the first step, transposon-end containing oligos (e.g., Oligo-01A-501 and Oligo-03A, or Oligo-01A-502 and Oligo-03A) were resuspend in annealing buffer (40 mM Tris-HCl (pH8.0), 50 mM NaCl) to stock concentration of 100 μM. In a PCR tube, 10 μl oligo-01A, 10 μl oligo-03A were mixed, vortexed and placed in PCR tubes in a thermocycler with the following program: 5 minutes at 95° C., cool to 65° C. (−0.1° C./second), 5 minutes at 65° C., cool to 25° C. (−0.1° C./second), 5 minutes at 25° C., and hold at 4° C.

Transposome complexes were assembled following the procedure below, as recommended by the manufacturer. The reagents were mixed in a PCR tube: 10 μL Tn5 transposase (2 ug/ul), 10 μL annealed adaptor. The reagents were mixed thoroughly and incubated at 23° C. for 30 minutes. 10 μl glycerol was added and mixed. The assembled transposome complex was stored at −20° C.

The workflow for this example is shown in FIG. 7. The tagmentation reaction was performed using genomic DNA extracted from gene edited samples or controls (using standard methods) with the assembled transposome complex using the following procedure: 100 ng genomic DNA from unedited control or edited sample, 20 μL 2× Tagmentation buffer, 200 ng loaded Tn5 Transposase, and added H2O up to 40 μL. The reaction mixtures were mixed thoroughly and incubated at 55° C. for 15 minutes. 10 μL 0.2% SDS was added. And then Tn5 was inactivated for 10 min at 70° C. The tagmented DNA was purified using a Zymo column following the manufacturer's instructions and eluted in 11 μL.

The 3′ ends of DNA molecules were then blocked using ddNTP and terminal transferase (TdT, NEB, M0315S) using the following procedure: 10 μL tagmented DNA, 5 μL 10× TdT buffer, 2 μL (40U) TdT, 5 μL CoCl2 solution, 1 μL ddNTP (10 mM), and added up to 50 μL. The reactions were incubated at 37° C. for 2 hours, 70° C. for 10 min, and cooled down to 4° C. The DNA was then purified using a Zymo column following the manufacturer's instructions and eluted in 21 μL.

Tagmented and 3′ blocked DNA from the previous step was used for two separate PCR reactions for each target, one reaction to detect genome rearrangement on the left-side of the target and the other reaction to detect genome rearrangement on the right-side of the target. Each PCR was then performed with a common primer annealed to the adapter region, a target specific blocker and a target specific primer. PCR was performed using a 4-step PCR cycling protocol with tagmented DNA, Primer 1 (Oligo-04A, common to all targets and rearrangement of both sides), Primer 2 (e.g., Oligo-19 for CCR5 #1 left-side rearrangement, or Oligo-23 for CCR5 #1 right-side rearrangement), and Blocker 0 (e.g., Oligo-05A for CCR5 #1 left-side rearrangement, or Oligo-09A for CCR5 #1 right-side rearrangement) using SuperFi II Polymerase PCR Master Mix (ThermoFisher, Cat #12368010). PCR mixture was prepared as below: 25 μL2× PCR Master Mix, 10 μL Tagmented DNA, 1.25 μL Primer 1 (10 μM), 2.5 μL Primer 2 (10 μM), 10 μL Blocker 0 (10 μM), and add H2O up to 50 μL. PCR was performed using the following program: initial denaturing at 98° C. for 30 seconds, 15 cycles of the 4-step denaturing-annealing-amplification (10 seconds at 98° C., 10 seconds at 78° C., 10 second at 60° C., 60 seconds at 72° C.), and final extension at 72° C. for 5 minutes, and hold at 4° C. PCR products were purified using AMpure XP beads (1×) and eluted in 12 μL.

Nested PCR was then performed with a barcode tag. PCR was performed with PCR product from the previous step, Primer 3 (Oligo-04A, common to all targets and rearrangement of both sides), Primer 4 (e.g., Oligo-20 for CCR5 #1 left-side rearrangement, or Oligo-24 for CCR5 #1 right-side rearrangement), and Blocker 0 (e.g., Oligo-05A for CCR5 #1 left-side rearrangement, or Oligo-09A for CCR5 #1 right-side rearrangement) using SuperFi II Polymerase PCR Master Mix (ThermoFisher, Cat #12368010). PCR mixture was prepared as below: 25 μL 2× PCR Master Mix, 10 μL PCR product, 1.25 μL Primer 3 (10 μM), 2.5 μL Primer 4 (10 μM), 10 μL Blocker 0 (10 μM), and add H2O up to 50 μL. PCR was performed using the following program: initial denaturing at 98° C. for 30 seconds, 15 cycles of the 4-step denaturing-annealing-amplification (10 seconds at 98° C., 10 seconds at 78° C., 10 second at 60° C., seconds at 72° C.), and final extension at 72° C. for 5 minutes, and hold at 4° C. PCR products were purified using AMpure XP beads, and eluted in 12 μL.

Tag PCR was then performed with sequencing primers. PCR was performed with PCR product from the previous step, Primer 1 (Oligo-04A, common to all targets and rearrangement of both sides), Primer 6 (e.g., any Oligo-13A, or Illumina indexing primer from New England Biolabs, NEB #E7611A), and Blocker 0 (e.g., Oligo-05A for CCR5 #1 left-side rearrangement, or Oligo-09A for CCR5 #1 right-side rearrangement) using SuperFi II Polymerase PCR Master Mix (ThermoFisher, Cat #12368010). PCR mixture was prepared as follows: 25 μL 2× PCR Master Mix, 10 μL PCR product, 5 μL Primer 5 (10 μM), 5 μL Primer 6 (10 μM), 5 μL Blocker 0 (20 μM). PCR is performed using the following program: initial denaturing at 98° C. for 30 seconds, 15 cycles of the 4-step denaturing-annealing-amplification (10 seconds at 98° C., 10 seconds at 78° C., 10 second at 60° C., seconds at 72° C.), and final extension at 72° C. for 5 minutes, and hold at 4° C. PCR products were purified using AMpure XP beads, and eluted in 52 μL.

50 μL of the supernatant was transferred to a new tube for library quantification using NEBNext library Quant Kit for Illumina (NEB, E7630) following the manufacture's instructions. Libraries were normalized and pooled for sequencing.

Next generation sequencing was then performed using the PCR product from the previous step, and sequencing was performed using an Illumina platform (NextSeq 2000) with 20% PhiX (Illumina, Cat #FC-110-3001).

Paired-end sequencing reads from Illumina sequencing were processed using a customized NGS analysis pipeline. The sequence reads were first checked for overall quality using FastQC followed by consolidating FastQC results using MultiQC. The reads were then subjected to Adapter and primer sequence trimming using Cutadapt (DOI:10.14806/ej.17.1.200) followed by trimming low quality bases using Trimmomatic (Bolger et al, Bioinformatics. 2014; 30 (15): 2114-20). The trimming in the reads was verified by running FastQC and MultQC steps on trimmed reads fastq files to ascertain the removal of adaptors. The resulting reads were aligned with human genome reference sequence (hg38) using BWA-MEM (Li and Durbin, Bioinformatics, 2009; 25:1754-1760) tool with default parameters. The generated Sequence Alignment Map (SAM) file was sorted by coordinate and converted to Binary Alignment Map (BAM) file using PicardTools (http://broadinstitute.github.io/picard). The BAM file was then filtered to remove low-quality reads and to keep the reads with quality score ≥30 using Samtools. The resulting high quality BAM file was then checked using the CollectInsertSizeMatrix and CollectAlignemntSummary Matrics modules from PicardTools followed by FastQC and MultiQC report generation. The reads in the sorted high quality read BAM were grouped based on UMIs using UMI-Tools with paired option and saved as grouped BAM followed by BAM indexing using Samtools. The grouped BAM file was then de-duplicated using UMI-Tools with paired option to remove PCR duplicate reads. The FGSV tool was then used to discover the structural variation pileup by searching for split read mapping and read pairs that map across breakpoints in the BAM file using the FGSV SVPileup module. The AggregateSvPileup module of FGSV then aggregated information across nearby pileups to call structural variants. Only the pileups containing at least one breakpoint on the target were considered as real hits, and as high confidence hits with at least 10 split reads. IGV (Thorvaldsdóttir et al, Brief Bioinform. 2013; 14:178-192) was used to facilitate the identification and analysis of rearrangements at genomic scale.

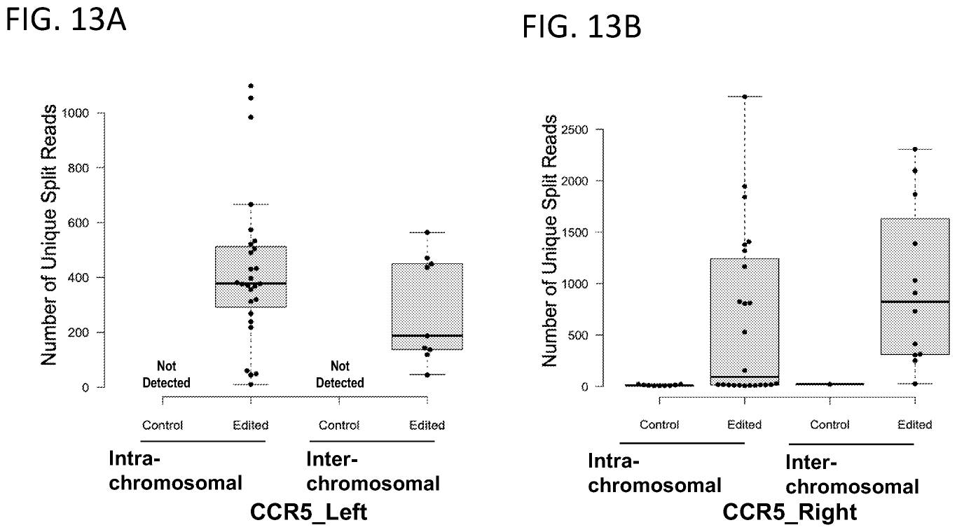

DNA rearrangement events detected for Example 4 in control and edited cells are shown in Table 11 below. The criteria used to filter out noise is the same with Method I_{A}. As shown in Table 11, 36 and 37 hits of high confidence passed the filters in edited cells, for the left-side and right-side DNA rearrangement events, respectively, while only 0 and 10 hits were captured in control cells for the left-side events, and right-side events, respectively. However, besides more hits, the numbers of the split reads to support these hits were also much higher in edited cells in FIGS. 13A-B.

TABLE 11
Summary of DNA rearrangement by Example
4 in control and CCR5 edited cell.

| Samples | Side | Intra Chromosomal | Inter Chromosomal |
|---|---|---|---|
| Control | Left | 0 | 0 |
| CCR5 edited | | 27 | 9 |
| Control | Right | 9 | 1 |
| CCR5 edited | | 25 | 12 |

Note:
DNA rearrangment events with split read number <10 were filtered out. One breakpoint must be located at ON target cutting site +/−25 bp.

DNA rearrangements identified by Example 4 in CCR5 edited cells are displayed in Table 12 below. The genomic coordinates of the DNA breakpoints between DNA rearrangements, number of unique split-reads based on UMI-tools, extracted reads to show the split reads, and note to further explain the potential mechanism for this DNA rearrangements are included in the Table 12. It is worth noting that the DNA rearrangements between CCR5 and its homologous gene CCR2, inter-chromosomal translocation between CCR5 on chromosome 3 and RNF17/CENPJ on chromosome 13 were also identified by CAST-seq (Turchiano et al, 2021). The presence of fused DNA of chr3/chr13 and acentric chromosome were also validated using PCR (FIGS. 13A-B). Therefore, SAFER detection Method I_{B} can efficiently capture intra- and inter-chromosomal rearrangements.

TABLE 12
Examples of DNA rearrangements identified by Example 4 in CCR5 edited cells.

| | | | | Unique | | |
|---|---|---|---|---|---|---|
| Cat- | | On-target | Non-target | Split_ | | |
| egory | Side | breakpoint | breakpoint | reads | Exemplar read | Note |
| Intra- | Left | chr3: | chr3: | 21 | Read_name: | The event |
| chromo- | | 46373001- | 46372167- | | VH01033: 21: AACCM73M5: | represents a |
| somal | | 46373011 | 46372170 | | 1: 1103: 21602: | large deletion |
| re- | | | | | 42423: rGCCTTGGGAG | caused by |
| arrange | | | | | Sequence: | CRISPR/Cas9 |
| ment | | | | | GAACAGTGATTGGCATCC | editing. |
| | | | | | AGTAGGTGCCCTCTAGGC | No off-target |
| | | | | | CTCCGCTCTACTCACTGG | site was |
| | | | | | TGTTCATCTTTGGTTTTGT | predicted at the |
| | | | | | GGGCAACATGCTGGTCAT | non-target |
| | | | | | CCTCATCCTGATAAACTG | breakpoint site. |
| | | | | | CAAAAGGCTGAAGAGC | Gene: CCR5 |
| | | | | | (SEQ ID NO: 73) | |
| | | | | | Alignments: | |
| | | | | | chr3: | |
| | | | | | 46,372,128-46,372,167 | |
| | | | | | (+) | |
| | | | | | chr3: | |
| | | | | | 46,373,005-46,373,091 | |
| | | | | | (+) | |
| | Left | chr3: | chr3: | 525 | Read name: | This event |
| | | 46373024- | 46357598- | | VH01033:21: AACCM73M5: | represents an |
| | | 46373032 | 46357604 | | 1: 1104: 14160: | intergenic |
| | | | | | 45016: rCTACCTTCCT | inversion that is |
| | | | | | Sequence: | caused by off- |
| | | | | | GCTCTTCAGCCTTTTGCA | target editing or |
| | | | | | GTTTATCAGGATGAGGAT | homology |
| | | | | | GACCAGCATGTTGCCCAC | mediated |
| | | | | | AAAACCAAAGATGATTA | recombination. |
| | | | | | TGATTACGGTGCTCCCTG | Predicted off- |
| | | | | | TCATAAATTTGACGTGAA | target site: |
| | | | | | GCAAATTGGGGCCCAACT | CACCAGCGA |
| | | | | | CCTGCCTCCGCTCTACTC | GTAGAGCGG |
| | | | | | CCTGGTGT (SEQ ID NO: | AGGCAGGAG |
| | | | | | 74) | (SEQ ID NO: 61) |
| | | | | | Alignments: | chr3: |
| | | | | | chr3: | 46357657- |
| | | | | | 46,373,026-46,373,091 | 46357683 and |
| | | | | | (−) | homology |
| | | | | | chr3: | sequence in |
| | | | | | 46,357,598-46,357,684 | CCR2 and |
| | | | | | (+) underlined | CCR5: |
| | | | | | | CTGGTGTTC |
| | | | | | | ATCTTTGGTT |
| | | | | | | TTGTGGGCA |
| | | | | | | ACATGCTGG |
| | | | | | | TC (SEQ ID |
| | | | | | | NO: 62) |
| | | | | | | Gene: CCR2, |
| | | | | | | CCR5 |
| | Right | chr3: | chr3: | 15 | Read name: | This event |
| | | 46372992- | 46357616- | | VH01033: 30: AAF5TJWM5: | represents an |
| | | 46373001 | 46357619 | | 1: 1101: 57788: | intergenic |
| | | | | | 4048: rCTAATAACCA | inversion that is |
| | | | | | Sequence: | caused by off- |
| | | | | | TACATCGGAGCCCTGCCA | target editing or |
| | | | | | AAAAATCAATGTGAAGC | homology |
| | | | | | AAATCGCAGCCCGCCTCC | mediated |
| | | | | | CTGTCATAAATTTGACGT | recombination. |
| | | | | | GAAGCAAATTGGGGCCC | Predicted off- |
| | | | | | AACTCCTGCC (SEQ ID | target site: |
| | | | | | NO: 75) | CACCAGCGA |
| | | | | | Alignments: | GTAGAGCGG |
| | | | | | chr3: | AGGCAGGAG |
| | | | | | 46,372,946-46,372,999 | (SEQ ID NO: |
| | | | | | (+) | 61) |
| | | | | | chr3: | chr3:46357657- |
| | | | | | 46,357,619-46,357.664 | 46357683 and |
| | | | | | (+) underlined | homology |
| | | | | | | sequence in |
| | | | | | | CCR2 and |
| | | | | | | CCR5: |
| | | | | | | CTGGTGTTC |
| | | | | | | ATCTTTGGTT |
| | | | | | | TTGTGGGCA |
| | | | | | | ACATGCTGG |
| | | | | | | TC (SEQ ID |
| | | | | | | NO: 62) |
| | | | | | | Gene: CCR2, |
| | | | | | | CCR5 |
| | Right | chr3: | chr3: | 1293 | Read_name: | Predicted off- |
| | | 46372987- | 46372579- | | VH01033: 30: AAF5TJWM5: | target site: |
| | | 46373001 | 46372588 | | 1: 1103: 14974: | CACCAGCGA |
| | | | | | 28678:rCGCCACCCCC | GTAGAGCGG |
| | | | | | Sequence: | AGGCAGGAG |
| | | | | | TACATCGGAGCCCTGCCA | (SEQ ID NO: |
| | | | | | AAAAATCAATGTGAAGC | 61) |
| | | | | | AAATCGCAGCCCGCCTCC | chr3:46357657- |
| | | | | | TGAAACTGGGTCAAGCAT | 46357683. |
| | | | | | GGTGCAGCTCAGCCTTTT | Intergenic |
| | | | | | TG (SEQ ID NO: 76) | inversion |
| | | | | | Alignments: | caused by off- |
| | | | | | chr3: | target editing or |
| | | | | | 46,372,946-46,373,000 | homology |
| | | | | | (+) | mediated |
| | | | | | chr3: | recombination. |
| | | | | | 46,372,547-46,372,584 | Gene: CCR2, |
| | | | | | (−) underlined | CCR5 |
| | Right | chr3: | chr3: | 1355 | Read name: | The event |
| | | 46372986- | 46373766- | | VH01033:30: AAF5TJWM5: | represents a |
| | | 46372991 | 46373769 | | 1: 2204: 19027: | large deletion |
| | | | | | 32162: rCAGTAGCGGG | caused by |
| | | | | | Sequence: | CRISPR/Cas9 |
| | | | | | TACATCGGAGCCCTGCCA | editing. |
| | | | | | AAAAATCAATGTGAAGC | No off-target |
| | | | | | AAATCGCAGCACTGCTGC | site was |
| | | | | | ATCAACCCCATCATCTAT | predicted at the |
| | | | | | GCCTTTGTCGGGGAGAAG | non-target |
| | | | | | TTCAGAAACTACCTCTTA | breakpoint site. |
| | | | | | GTC (SEQ ID NO: 77) | Gene: CCR5 |
| | | | | | Alignments | |
| | | | | | chr3: | |
| | | | | | 46,372,946-46,372,990 | |
| | | | | | (+) | |
| | | | | | chr3: | |
| | | | | | 46,373,766-46,373,832 | |
| | | | | | (+) underlined | |
| Inter | Left | chr3: | chr13: | 15 | Read_name: | These events |
| chromo | | 46373002- | 32488626- | | VH01033:21: AACCM73M5: | represent |
| somal | | 46373003 | 24886326 | | 1: 2103: 56102: | interchromosomal |
| re- | | | | | 51699: rACCGGATCGC | translocation |
| arrange | | | | | Sequence: | that is mediated |
| ment | | | | | GATGAGGATGACCAGCA | by Off-target |
| | | | | | TGTTGCCCACAAAACCAT | editing. |
| | | | | | AGATGAACACCAGTGAG | On-target site: |
| | | | | | TAGAGCGGAGAGAGGTG | chr3:46372995- |
| | | | | | GTCAGCCAACAAGCTGCT | 46373017 (−) |
| | | | | | GGGCGTGTGAGGCGGCT | GTGAGTAGA |
| | | | | | GTGCTGTGCCTGTGAGAC | GCGGAGGCA |
| | | | | | AGGAATGGTTCCATTACA | GGAGG (SEQ |
| | | | | | C (SEQ ID NO: 78) | ID NO: 30) |
| | | | | | Alignments | OFF-target site: |
| | | | | | chr3: | G()GAGGAGA |
| | | | | | 46,373,003-46,373,064 | GCGGAGGCA |
| | | | | | (-) | GGTGG |
| | | | | | chr13: | Chr13: 24886320- |
| | | | | | 24,886,326-24,886,413 | 24886342 (+) |
| | | | | | (+) underlined | Gene: CCR5 |
| | Right | chr3: | chr13: | 418 | Read name: | and |
| | | 46372995- | 24886335- | | VH01033:30: AAF5TJWM5: | RNF17/CENPJ |
| | | 46373002 | 24886340 | | 1: 1302: 22757: | |
| | | | | | 39640: rTGAACCGGGG | |
| | | | | | Sequence: | |
| | | | | | TACATCGGAGCCCTGCCA | |
| | | | | | AAAAATCAATGTGAAGC | |
| | | | | | AAATCGCAGCCCGCCTCC | |
| | | | | | TAGGTGGTCAGCCAACA | |
| | | | | | AGCTGCTGGGCGTGTGAG | |
| | | | | | GCGGCTGTGCTGTGCCTG | |
| | | | | | TGAGACAGGAATGGTTCC | |
| | | | | | ATTACAC (SEQ ID NO: 79) | |
| | | | | | Alignments: | |
| | | | | | chr3: | |
| | | | | | 46,372,946-46,373,000 | |
| | | | | | (+) | |
| | | | | | chr13: | |
| | | | | | 24,886,336-24,886,413 | |
| | | | | | (+) underlined | |

### Example 5: Exemplary Method for Method I_{C}

Exemplary oligonucleotides are listed in Table 1.

Additional reagents include Tn5 transposase, e.g., Robust Tn5 Transposase (Cat #EMQZ1422, Creative Biogene), DNA polymerase PCR master mix, e.g., Platinum™ SuperFi II PCR Master Mix (ThermoFisher, Cat #12368010), DNA polymerase with PCR buffer, e.g., Platinum™ SuperFi II DNA Polymerase (ThermoFisher, Cat #12361010), dNTP, e.g., dNTP Mix (10 mM each) (ThermoFisher, Cat #R0194), DNA purification kit: Column kit, e.g., DNA Clean & Concentrator-5 (Zymo Research, Cat #D4004), Magnetic beads, e.g., AMPure XP Reagent (Beckman, Cat #A63880), Illumina sequencing kit, e.g., NextSeq 1000/2000 P1 Reagents (300 Cycles) (Cat #20050264), MiSeq Reagent Kit v3 (150-cycle) (Cat #MS-102-3001), MiSeq Reagent Kit v2 (300-cycles) (Cat #MS-102-2002), PhiX Control v3 (Illumina, Cat #FC-110-3001), USER® Enzyme (New England Biolabs, Cat #M5505S), STE buffer (10 mM Tris-HCl (pH 8.0), 1 mM EDTA, 0.1M NaCl).

In the first step, Tn5 transposomes are assembled. Transposon-end containing oligos (e.g., Oligo-10 and Oligo-02 for non-UMI version, or Oligo-27 and Oligo-02 for UMI version) are annealed in STE buffer with the following program: 1-10 minutes at 90-99° C., 40-500 cycles of 1 minutes at 90-99° C. (decrease temperature 0.2-2° C. every cycle until reaching 1-12° C.). Transposome complexes are assembled in TPS buffer following the procedure below, as recommended by the manufacturer (Creative Biogene, Cat #EMQZ1422). The reagents are mixed thoroughly and incubated at 15-40° C. for 5-120 minutes.

| Component | Volume | |
|---|---|---|
| Tn5 transposase | 1-10 | μL |
| Adaptor | 0.5-8 | μL |
| 10x TPS buffer | 2 | μL |

| | |
|---|---|
| Sterile water | add to 20 μL |

The workflow for this example is shown in FIG. 3. The tagmentation reaction is performed in which genomic DNA extracted from gene edited samples or controls (using standard methods) is tagmented with the assembled transposome complex following procedure. An exemplary reaction mixture is shown below:

| Component | Volume | |
|---|---|---|
| Genomic DNA | varies (5-500 ng) | |
| 5x LM buffer | 6 | μL |
| Transposome | 0.1-8 | μL |

| | |
|---|---|
| Sterile water | add to 30 μL |

The reaction components are mixed thoroughly and incubated at 30-65° C. for 5-120 minutes. The tagmented DNA is purified using column-based purification.

First new strand synthesis is then performed. The PCR reaction is set up with fragmented DNA from the previous step, Primer 2 (e.g., Oligo-05 for EMX1 left-side rearrangement, Oligo-15 for EMX1 right-side rearrangement, Oligo-19 for CCR5 #1 left-side rearrangement, or Oligo-23 for CCR5 #1 right-side rearrangement), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: denaturing at 90-99° C. for 1-10 minutes, blocker annealing at 75-92° C. for 5-120 seconds, primer annealing at 55-75° C. for 1 minute, primer extension at 67-75° C. for 1-10 minutes, and hold at 4-12° C. Product is purified using column-based purification.

USER enzyme cleavage is then performed. Briefly, USER enzyme cleavage is performed with the following procedure: 0.5-10 μL of USER enzyme is added to the product from the previous step, mixed and incubated at 16-45° C. for 5-60 minutes. Product is purified using a DNA purification kit.

USER treated DNA from the previous step is used for two separate PCR reactions for each target, one reaction to detect genome rearrangement on the left-side of the target and the other reaction to detect genome rearrangement on the right-side of the target. Each PCR is then performed with a common primer annealed to the adapter region, a target specific blocker and a target specific primer. PCR is performed using 3-step PCR cycling protocol with tagmented DNA, Primer 1 (Oligo-04, common to all targets and rearrangement of both sides), Primer 2 (e.g., Oligo-05 for EMX1 left-side rearrangement, Oligo-15 for EMX1 right-side rearrangement, Oligo-19 for CCR5 #1 left-side rearrangement, or Oligo-23 for CCR5 #1 right-side rearrangement), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: initial denaturing at 90-99° C. for 1-5 minute, 20-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Nested PCR is then performed with barcode tag. PCR is performed with the PCR product from the previous step, Primer 3 (Oligo-06, common to all targets and rearrangement of both sides), Primer 4 (e.g., Oligo-07 for EMX1 left-side rearrangement, Oligo-16 for EMX1 right-side rearrangement, Oligo-20 for CCR5 #1 left-side rearrangement, or Oligo-24 for CCR5 #1 right-side rearrangement), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: initial denaturing at 90-99° C. for 1-5 minute, 4-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Tag PCR is then performed with sequencing primers. PCR is performed with PCR product from the previous step, Primer 5 (e.g., Oligo-08, an example of Illumina indexing primer from New England Bio Labs, NEB #E7603A), Primer 6 (e.g., Oligo-09, an example of Illumina indexing primer from New England Bio Labs, NEB #E7611A), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: initial denaturing at 90-99° C. for 1-5 minute, 4-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Next generation sequencing is then performed using the PCR product from the previous step, and sequencing is performed using an Illumina platform (e.g., Illumina MiSeq or NextSeq 2000) with optional >=0-95% PhiX (Illumina, Cat #FC-110-3001).

Paired-end sequencing reads from Illumina sequencing are merged using a paired-end reads merging software, e.g., PEAR (Zhang et al, Bioinformatics. 2014; 30:614-620), FLASH (Magoč and Salzberg, Bioinformatics. 2011; 27:2957-2963), BBMerge (Bushnell et al, PLoS One. 2017; 12:e0185056), or NGmerge (Gaspar, Bioinformatics. 2018; 19:536). The merged reads are then trimmed and filtered using software such as BBmap (https://jgi.doe.gov/data-and-tools/software-tools/bbtools/), samtools (Danecek et al, Gigascience. 2021; 10. doi: 10.1093/gigascience/giab008), or custom scripts to remove Illumina adaptor sequences, low quality reads, reads containing the target flanking sequence on the side of the target site where the blocking oligonucleotide or cleavage reagent binds, and reads that do not contain the target flanking sequence on the side of the target site where the target-specific primer hybridizes. Selected reads are aligned to a human reference genome, e.g., GRCh37 (hg19), GRCh38 (hg38), or Telomere-to-Telomere assembly (e.g., T2T-CHM13v2.0), using an alignment software, e.g., Bowtie 2 (Langmead and Salzberg, Nat Methods. 2012; 9:357-359), BWA (Li and Durbin, Bioinformatics. 2009; 25:1754-1760), or Minimap2 (Li, Bioinformatics. 2018; 34:3094-3100). The aligned BAM file is converted into bed file using BEDTools (Quinlan, Bioinformatics. 2014; 47:11.12.1-34). For methods using UMIs, UMIs are collapsed to remove redundant sequencing reads using software such as Gencore (Chen et al, Bioinformatics. 2019; 20:606), UMI-tools (Smith et al, Genome Res. 2017:27: 491-499), UMI-Reducer (github.com/smangul1/UMI-Reducer), or custom scripts. Reads with candidate translocation break points within a suitable window flanking the target site (e.g., within 1, 3, 5, 10, 20, 40, 60, 80, 100, 200, or more bases) are identified and counted to quantify the number of rearrangements between the target site and other genomic loci. Statistical tests are then applied to establish a confidence score to each group of rearrangements at each distal rearranged locus (e.g., within a defined window of up 50, 100, 200, 500, 1000, 2000, 3000, or more bases at the distal rearranged locus). Visualization tools such as IGV (Thorvaldsdóttir et al, Brief Bioinform. 2013; 14:178-192) or Circos (Krzywinski et al, Genome Res. 2009; 19:1639-1645) are used to facilitate the identification and analysis of rearrangements at genomic scale.

### Example 6: Exemplary Method for Method I_{C}

Exemplary oligonucleotides used are listed in Table 5.

Additional reagents included unloadedTn5 Transposase (Diagenode, C01070010, Diagenode), 2× Tagmentation buffer (Diagenode, C01019043), Platinum™ SuperFi II PCR Master Mix (ThermoFisher, Cat #12368010), dNTP Mix (10 mM each) (ThermoFisher, Cat #R0194), DNA purification kit: DNA Clean & Concentrator-5 (Zymo Research, Cat #D4004), AMPure XP Reagent (Beckman Coulter, Cat #A63880), Glycerol (MilliporeSigma, 356350500ML), 10% SDS (Fisher, BP2436200), 5M NaCl (Invitrogen, AM9760G), Tris HCl 1M, pH8.0 (Invitrogen, 15568-025), Genomic DNA (extracted from edited and unedited 293T cells), ddNTP, Dideoxynucleoside Triphosphate Set (MilliporeSigma, Cat #03732738001), NEBNext library Quant Kit for Illumina (NEB, E7630), Terminal transferase (TdT, NEB, M0315S), USER, (NEB #M5508), Phusion U Hot Start DNA Polymerase (Thermofisher, F555S), Illumina NextSeq 1000/2000 P1 Reagents (300 Cycles) (Illumina, Cat #20050264), PhiX Control v3 (Illumina, Cat #FC-110-3001).

In the first step, transposon-end containing oligos (e.g., Oligo-02A and Oligo-03A) were resuspend in annealing buffer (40 mM Tris-HCl (pH8.0), 50 mM NaCl) to stock concentration of 100 μM. In a PCR tube, 10 μl oligo-01A, 10 μl oligo-03A were mixed, vortexed and placed in a thermocycler with the following program: 5 minutes at 95° C., cool to 65° C. (−0.1° C./second), 5 minutes at 65° C., cool to 25° C. (−0.1° C./second), 5 minutes at 25° C., and hold at 4° C.

Transposome complexes were assembled using the following procedure, as recommended by the manufacturer. The reagents were mixed in a PCR tube: 10 μL Tn5 transposase (2 ug/ul), 10 μL annealed adaptor. The reagents were mixed thoroughly and incubated at 23° C. for 30 minutes. 10 μl glycerol was added and mixed. The assembled transposome complex was stored at −20° C.

The workflow for this example is shown in FIG. 8. The tagmentation reaction was performed using genomic DNA extracted from gene edited samples or controls (using standard methods) with the following procedure:

100 ng genomic DNA from unedited control or edited sample, 20 μL 2× Tagmentation buffer, 200 ng loaded Tn5 Transposase, and add H2O up to 40 μL. The reaction mixtures were mixed thoroughly and incubated at 55° C. for 15 minutes. 10 μL 0.2% SDS is added. And then Tn5 was inactivated for 10 min at 70° C. The tagmented DNA was purified using a Zymo column following the manufacturer's instructions and eluted in 11 μL.

The 3′ ends of DNA molecules were then blocked using ddNTP and terminal transferase (TdT, NEB, M0315S) using the following procedure: 10 μL tagmented DNA, 5 μL 10× TdT buffer, 2 μL (40U) TdT, 5 μL CoCl2 solution, 1 μL ddNTP (10 mM), and added H2O up to 50 μL. The reactions were incubated at 37° C. for 2 hours, 70° C. for 10 min, and cooled down to 4° C. The DNA was then purified using a Zymo column following the manufacturer's instructions and eluted in 21 μL.

First strand synthesis was then performed using DNA from the previous step in two separate PCR reactions for each target, one reaction to detect genome rearrangement on the left-side of the target and the other reaction to detect genome rearrangement on the right-side of the target. Each PCR is then performed with a common primer annealed to the adapter region, a target specific blocker and a target specific primer. The PCR reaction was set up with fragmented and 3′ blocked DNA from the previous step, Primer 2 (e.g., Oligo-19 for CCR5 #1 left-side rearrangement, or Oligo-23 for CCR5 #1 right-side rearrangement), and Blocker 0 (e.g., Oligo-05A for CCR5 #1 left-side rearrangement, or Oligo-09A for CCR5 #1 right-side rearrangement) using Phusion U Hot Start DNA Polymerase (Thermofisher, F555S). The reaction mixture was prepared as follows: 10 μL Blocked DNA, 10 μL 5× Phusion buffer, 0.5 μL Phusion U polymerase, 1 μL dNTP (10 mM), 2.5 μL Primer 2 (10 μM), 10 μL Blocker-0 (10 μM), and add H2O up to 50 μL. PCR was performed using the following program: initial denaturing at 98° C. for 30 seconds, 1 or 20 cycles of the 4-step denaturing-annealing-amplification (10 seconds at 98° C., 10 seconds at 78° C., 10 second at 60° C., 60 seconds at 72° C.), and final extension at 72° C. for 5 minutes, and hold at 4° C. PCR products were purified using AMpure XP beads (1×) and eluted in 12 μL.

USER enzyme cleavage was then performed. Briefly, USER enzyme cleavage was performed using the following procedure: 10 μL Purified DNA, 5 μL (10×) rCutsmart buffer, 2 μL USER, and add H2O up to 50 μL. The mixture was incubated at 37° C. for 30 minutes. The DNA was purified using Zymo columns and eluted in 11 μl for PCR

USER treated DNA from the previous step was used for the first PCR amplification. Each PCR was performed with a common primer annealed to the adapter region, a target specific blocker and a target specific primer. PCR was performed using 4-step PCR cycling protocol with tagmented DNA, Primer 1 (Oligo-04A, common to all targets and rearrangement of both sides), Primer 2 (e.g., Oligo-06A for CCR5 #1 left-side rearrangement, or Oligo-10A for CCR5 #1 right-side rearrangement), and Blocker 0 (e.g., Oligo-05A for CCR5 #1 left-side rearrangement, or Oligo-09A for CCR5 #1 right-side rearrangement) using SuperFi II Polymerase PCR Master Mix (ThermoFisher, Cat #12368010). PCR mixture was prepared as below: 25 μL2× PCR Master Mix, 10 μL Tagmented DNA, 1.25 μL Primer 1 (10 μM), 2.5 μL Primer 2 (10 μM), 10 μL Blocker 0 (10 μM), and add H2O up to 50 μL. PCR was performed using the following program: initial denaturing at 98° C. for 30 seconds, 15 cycles of the 4-step denaturing-annealing-amplification (10 seconds at 98° C., 10 seconds at 78° C., 10 second at 60° C., 60 seconds at 72° C.), and final extension at 72° C. for 5 minutes, and hold at 4° C. PCR products were purified using AMpure XP beads (1×) and eluted in 12 μL.

Nested PCR was then performed with a barcode tag. PCR was performed using the PCR products from the previous step: Primer 3 (Oligo-04A, common to all targets and rearrangement of both sides), Primer 4 (e.g., Oligo-20 for CCR5 #1 left-side rearrangement, or Oligo-24 for CCR5 #1 right-side rearrangement), and Blocker 0 (e.g., Oligo-05A for CCR5 #1 left-side rearrangement, or Oligo-09A for CCR5 #1 right-side rearrangement) using SuperFi II Polymerase PCR Master Mix (ThermoFisher, Cat #12368010). The PCR mixture was processed as follows: 25 μL 2× PCR Master Mix, 10 μL PCR product, 1.25 μL Primer 3 (oligo-04, 10 μM), 2.5μ L Primer 4 (10 μM), 10 μL Blocker 0 (10μ), and add H2O up to 50 μL. PCR is performed using the following program: initial denaturing at 98° C. for 30 seconds, 15 cycles of the 4-step denaturing-annealing-amplification (10 seconds at 98° C., 10 seconds at 78° C., 10 second at 60° C., seconds at 72° C.), and final extension at 72° C. for 5 minutes, and hold at 4° C. PCR products were purified using AMpure XP beads, and eluted in 12 μL.

Tag PCR was then performed with sequencing primers. PCR was performed with the product from the previous step, Primer 5 (Oligo-04A, common to all targets and rearrangement of both sides), Primer 6 (e.g., any Oligo-13A, or Illumina indexing primer from New England Biolabs, NEB #E7611A), and Blocker 0 (e.g., Oligo-05A for CCR5 #1 left-side rearrangement, or Oligo-09A for CCR5 #1 right-side rearrangement) using SuperFi II Polymerase PCR Master Mix (ThermoFisher, Cat #12368010). The PCR mixture was prepared as follows: 25 μL 2× PCR Master Mix, 10 μL PCR product, 5 μL Primer 5 (10 μM), 5 μL Primer 6 (10 μM), 5 μL Blocker 0 (20 μM). PCR was performed using the following program: initial denaturing at 98° C. for 30 seconds, 15 cycles of the 4-step denaturing-annealing-amplification (10 seconds at 98° C., 10 seconds at 78° C., 10 second at 60° C., seconds at 72° C.), and final extension at 72° C. for 5 minutes, and hold at 4° C. The PCR products were purified using AMpure XP beads, and eluted in 52 μL.

50 μL of the supernatant was transferred to a new tube for library quantification using the NEBNext library Quant Kit for Illumina (NEB, E7630) following the manufacture's instructions. The libraries were normalized and pooled for sequencing.

Next generation sequencing was then performed using the PCR product from the previous step, and sequenced using an Illumina platform (NextSeq 2000) with 20% PhiX (Illumina, Cat #FC-110-3001).

Paired-end sequencing reads from Illumina sequencing were processed using a customized NGS analysis pipeline. The sequence reads were first checked for overall quality using FastQC followed by consolidating FastQC results using MultiQC. The reads were then subjected to Adapter and primer sequence trimming using Cutadapt (DOI:10.14806/ej.17.1.200) followed by trimming low quality bases using Trimmomatic (Bolger et al, Bioinformatics. 2014; 30(15):2114-20). The trimming in the reads was verified by running FastQC and MultQC steps on trimmed reads fastq files to ascertain the removal of adaptors. The resulting reads were aligned with human genome reference sequence (hg38) using BWA-MEM (Li and Durbin, Bioinformatics, 2009; 25:1754-1760) tool with default parameters. The generated Sequence Alignment Map (SAM) file was sorted by coordinate and converted to Binary Alignment Map (BAM) file using PicardTools (http://broadinstitute.github.io/picard). The BAM file was then filtered to remove low-quality reads and to keep the reads with quality score ≥30 using Samtools. The resulting high quality BAM file was then checked using the CollectInsertSizeMatrix and CollectAlignemntSummary Matrics modules from PicardTools followed by FastQC and MultiQC report generation. The reads in the sorted high quality read BAM were grouped based on UMIs using UMI-Tools with paired option and saved as grouped BAM followed by BAM indexing using Samtools. The grouped BAM file was then de-duplicated using UMI-Tools with paired option to remove PCR duplicate reads. The FGSV tool was then used to discover the structural variation pileup by searching for split read mapping and read pairs that map across breakpoints in the BAM file using the FGSV SVPileup module. The AggregateSvPileup module of FGSV then aggregated information across nearby pileups to call structural variants. Only the pileups containing at least one breakpoint on the target were considered as real hits, and as high confidence hits with at least 10 split reads. IGV (Thorvaldsdóttir et al, Brief Bioinform. 2013; 14:178-192) was used to facilitate the identification and analysis of rearrangements at genomic scale.

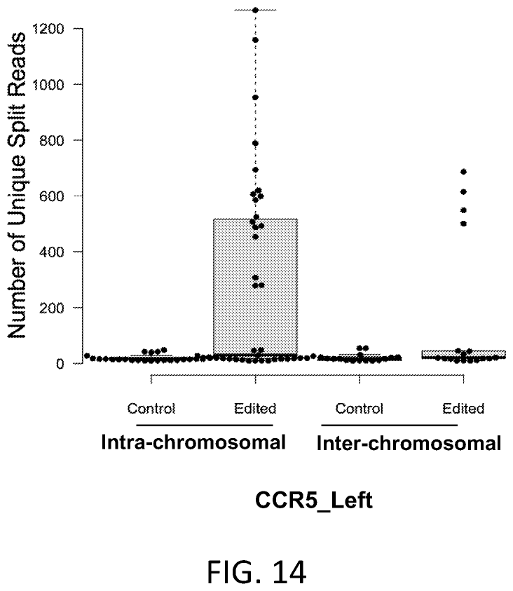

DNA rearrangement by Example 6 in control and CCR5 edited cells are shown in Table 13 below. The criteria used to filter out noise is the same with Method I_{A}. For this example, only libraries for left-side events were performed. As shown in Table 13, 38 and 57 hits of high confidence passed the filters in control and in edited cells, respectively. However, the number of unique split support these hits were still substantially higher in edited cells (FIG. 14).

TABLE 13
Summary of DNA rearrangement by Example
6 in control and CCR5 edited cell.

| Samples | Side | Intra Chromosomal | Inter Chromosomal |
|---|---|---|---|
| Control | Left | 22 | 16 |
| CCR5 edited | | 40 | 17 |

Note:
DNA rearrangment event with split read number <10 were filtered out. One breakpoint must be located at ON target cutting site +/−25 bp. Only the DNA rearrangements of the left side was examined.

DNA rearrangements identified by Example 6 in CCR5 edited cells are displayed in Table 14 below. The genomic coordinates of the DNA breakpoints between DNA rearrangements, number of unique split-reads based on UMI-tools, extracted reads to show the split reads, and note to further explain the potential mechanism for this DNA rearrangements were included in the table. It is worth noting that the inter-chromosomal translocation between CCR5 on chromosome 3 and RNF17/CENPJ on chromosome 13 were also identified by CAST-seq (Turchiano et al, 2021). The presence of fused DNA of chr3/chr13 and acentric chromosome were also validated using PCR (FIG. 13A-B). Therefore, SAFER detection Method I_{C} can efficiently capture intra- and inter-chromosomal rearrangements.

TABLE 14
Examples of DNA rearrangements identified by Example 6 in CCR5 edited cells.

| | | | | Unique | | |
|---|---|---|---|---|---|---|
| Cat- | | On-target | Non-target | Split_ | | |
| egory | Side | breakpoint | breakpoint | reads | Exemplar read | Note |
| Intra | Left | chr3: | chr3: | 39 | Read name: | The event |
| chromo | | 46373001- | 46372462- | | VH01033: 21: AACCM73M5: | represents a large |
| somal | | 46373004 | 46372467 | | 1: 1204: 40178: | deletion caused by |
| re- | | | | | 38655: rGGATTCACCT | CRISPR/Cas9 |
| arrange | | | | | Sequence: | editing. |
| ment | | | | | CTGTAGTCCCCAGCCACTTGG | No off-target site |
| | | | | | AGGGGTGAGGTGAGAGGATT | was predicted at |
| | | | | | GCTTGAGCCCGGGATGGTCCA | the non-target |
| | | | | | GGCTCCGCTCTACTCACTGGT | breakpoint site. |
| | | | | | GTTCATCTTTGGTTATGTGGGC | Gene: CCR5 |
| | | | | | AA (SEQ ID NO: 80) | |
| | | | | | Alignments: | |
| | | | | | chr3: | |
| | | | | | 46.372.401-46.372.465 | |
| | | | | | (+) underlined | |
| | | | | | chr3: | |
| | | | | | 46,373,003-46,373,046 (+) | |
| | Left | chr3: | chr3: | 706 | Read name: | |
| | | 46373002- | 46376507- | | VH01033: 21: AACCM73M5: | The event |
| | | 46373011 | 46376515 | | 1: 2204: 49532: | represents a large |
| | | | | | 17527:rCAACTAAAAG | deletion caused by |
| | | | | | Sequence: | CRISPR/Cas9 |
| | | | | | GCTCTTCAGCCTTTTGCAGTTT | cditing. This |
| | | | | | ATCAGGATGAGGATGACCAGC | intragenic |
| | | | | | ATGTTGCCCACAAAACCAAAG | Inversion may be |
| | | | | | ATGAACACTCAAAAGAATTGC | occurred between |
| | | | | | CATGTTTTAAAAAAGAGAATT | on-target CRISPR |
| | | | | | GGTGCCATAAGTTAGTTGTCT | editing and |
| | | | | | ATGTTTGAAAATGAAGAAGAT | naturally |
| | | | | | AT (SEQ ID NO: 81) | happened |
| | | | | | Alignments: | breakpoint. |
| | | | | | chr3: | No off-target site |
| | | | | | 46,373,002-46,373,047 (−) | was predicted at |
| | | | | | chr3: | the non-target |
| | | | | | 46,376,506-46,376,586 (+) | breakpoint site. |
| | | | | | underlined | Gene: CCR5 |
| Inter | Left | chr3: | chr13: | 21 | Read name: | This event |
| chromo | | 46373005- | 24886338- | | VH01033: 21: AACCM73M5: | represents an |
| somal | | 46373006 | 24886339 | | 1: 2403: 34061: | interchromosomal |
| re- | | | | | 51586: rAACAGCTTGT | translocation that |
| arrange | | | | | Sequence: | is mediated by |
| ment | | | | | GATGAGGATGACCAGCATGTT | Off-target editing. |
| | | | | | GCCCACAAAACCAAAGATGA | On-target site: |
| | | | | | ACACCAGTGAGTAGAGCGTGG | chr3:46372995- |
| | | | | | TCAGCCAACAAGCTGCTGGGC | 46373017 (−) |
| | | | | | GTGTGAGGCGGCTGTGCTGTG | GTGAGTAGAG |
| | | | | | CCTGTGAGACAGGAATGGTTC | CGGAGGCAGG |
| | | | | | CATTACACCATGGGAAATCAC | AGG (SEQ ID |
| | | | | | (SEQ ID NO: 82) | NO: 30) |
| | | | | | Alignments: chr3: | OFF-target site: |
| | | | | | 46,373,005- | G()GAGGAGAG |
| | | | | | 46,373,064 (-) | CGGAGGCAGG |
| | | | | | chr13: | TGG |
| | | | | | 24,886,340-24,886,426 (+) | Chr13: |
| | | | | | | 24886320- |
| | | | | | | 24886342 (+) |
| | | | | | | Gene: CCR5 and |
| | | | | | | RNF17/CENPJ |

### Example 7: Exemplary Method for Method II

Exemplary oligonucleotides are listed in Table 1.

Additional reagents include Tn5 transposase, e.g., Robust Tn5 Transposase (Cat #EMQZ1422, Creative Biogene), DNA polymerase PCR master mix, e.g., Platinum™ SuperFi II PCR Master Mix (ThermoFisher, Cat #12368010), DNA polymerase with PCR buffer, e.g., Platinum™ SuperFi II DNA Polymerase (ThermoFisher, Cat #12361010), dNTP, e.g., dNTP Mix (10 mM each) (ThermoFisher, Cat #R0194), DNA purification kit: Column kit, e.g., DNA Clean & Concentrator-5 (Zymo Research, Cat #D4004), Magnetic beads, e.g., AMPure XP Reagent (Beckman, Cat #A63880), Illumina sequencing kit, e.g., NextSeq 1000/2000 P1 Reagents (300 Cycles) (Cat #20050264), MiSeq Reagent Kit v3 (150-cycle) (Cat #MS-102-3001), MiSeq Reagent Kit v2 (300-cycles) (Cat #MS-102-2002), PhiX Control v3 (Illumina, Cat #FC-110-3001), Sequence-specific nuclease and ribonucleoprotein assembly buffer, e.g., Cas9 Nuclease, S. pyogenes (New England Biolabs, Cat #M0386S), EnGen® Lba Cas12a (Cpf1) (New England Biolabs, Cat #M0653S), SpRY, Guide RNA specific to target region, STE buffer (10 mM Tris-HCl (pH 8.0), 1 mM EDTA, 0.1M NaCl).

Exemplary guide RNA sequences are shown in Table 15 below.

TABLE 15

| Design | Sequence (PAM is underlined, | Coordinate in hg38 |
|---|---|---|
| group | not included in sgRNA) | (chr strand start end) |
| EMX1_L | CAAACGGCAGAAGCTGGAGGAGG | chr2 + 72933822 72933844 |
| | (SEQ ID NO: 31) | |
| EMX1_R | CTCCCCATTGGCCTGCTTCGTGG | chr2 − 72933905 72933927 |
| | (SEQ ID NO: 32) | |
| CCR5_L | TGACATCAATTATTATACATCGG | chr3 + 46372932 46372954 |
| | (SEQ ID NO: 33) | |
| CCR5_R | TTTTGCAGTTTATCAGGATGAGG | chr3 − 46373058 46373080 |
| | (SEQ ID NO: 34) | |

In the first step, Tn5 transposomes are assembled. Transposon-end containing oligos (e.g., Oligo-01 and Oligo-11 for non-UMI version, or Oligo-26 and Oligo-11 for UMI version) are annealed in STE buffer with the following program: 1-10 minutes at 90-99° C., 40-500 cycles of 1 minutes at 90-99° C. (decrease temperature 0.2-2° C. every cycle until reaching 1-12° C.). Transposome complexes are assembled in TPS buffer following the procedure below, as recommended by the manufacturer (Creative Biogene, Cat #EMQZ1422). The reagents are mixed thoroughly and incubated at 15-40° C. for 5-120 minutes.

| Component | Volume | |
|---|---|---|
| Tn5 transposase | 1-10 | μL |
| Adaptor | 0.5-8 | μL |
| 10x TPS buffer | 2 | μL |

| | |
|---|---|
| Sterile water | add to 20 μL |

The workflow for this example is shown in FIG. 4. The tagmentation reaction is performed in which genomic DNA extracted from gene edited samples or controls (using standard methods) is tagmented with the assembled transposome complex following procedure. An exemplary reaction mixture is shown below:

| Component | Volume | |
|---|---|---|
| Genomic DNA | varies (5-500 ng) | |
| 5x LM buffer | 6 | μL |
| Transposome | 0.1-8 | μL |

| | |
|---|---|
| Sterile water | add to 30 μL |

The reaction components are mixed thoroughly and incubated at 30-65° C. for 5-120 minutes. The tagmented DNA is purified using column-based purification.

Two separate ribonucleoprotein (RNP) assembly reactions and corresponding in vitro cleavage (IVC) reactions are then performed for each target, one reaction to detect genome rearrangement on the left-side of the target and the other reaction to detect genome rearrangement on the right-side of the target. CRISPR/Cas9 Nuclease, S. pyogenes (New England Biolabs, Cat #M0386S) and sgRNA (Table 5) are assembled into RNP with the following procedure:

| | |
|---|---|
| NEBuffer r3.1 | 3 μL |
| 300 nM sgRNA | 0.6-15 μL (6-150 nM final) |
| 1 μM Cas9 Nuclease | 0.2-5 μL (6-150 nM final) |
| Sterile water | add to 30 μL |

The reaction mix is mixed thoroughly and incubated at 15-45° C. for 1-120 minutes. RNP is added to the tagmented DNA at an RNP: DNA ratio of 0.01:1 to 50:1. Cleavage reaction is incubated at 15-45° C. for 5-120 minutes. Product is purified using column-based purification.

Optionally, the 3′ ends of DNA molecules are then blocked. 3′ ends of DNA molecules are blocked using ddNTP and DNA polymerase SuperFi II (ThermoFisher, Cat #12361010). Product is purified using column-based purification.

PCR using IVC DNA from the previous step is then performed with a common primer annealed to the adapter region and a target specific primer. PCR is performed using 3-step PCR cycling protocol with IVC DNA, Primer 1 (Oligo-04, common to all targets and rearrangement of both sides) and Primer 2 (e.g., Oligo-05 for EMX1 left-side rearrangement, Oligo-15 for EMX1 right-side rearrangement, Oligo-19 for CCR5 #1 left-side rearrangement, or Oligo-23 for CCR5 #1 right-side rearrangement) using SuperFi II Polymerase PCR Master Mix (ThermoFisher, Cat #12368010), with the following program: initial denaturing at 90-99° C. for 1-5 minute, 20-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Nested PCR is then performed with barcode tag. PCR is performed with PCR product from the previous step, Primer 3 (Oligo-06, common to all targets and rearrangement of both sides), Primer 4 (e.g., Oligo-07 for EMX1 left-side rearrangement, Oligo-16 for EMX1 right-side rearrangement, Oligo-20 for CCR5 #1 left-side rearrangement, or Oligo-24 for CCR5 #1 right-side rearrangement), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: initial denaturing at 90-99° C. for 1-5 minute, 4-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Tag PCR is then performed with sequencing primers. PCR is performed with PCR product from the previous step, Primer 5 (e.g., Oligo-08, an example of Illumina indexing primer from New England Bio Labs, NEB #E7603A), Primer 6 (e.g., Oligo-09, an example of Illumina indexing primer from New England Bio Labs, NEB #E7611A), and Blocker 0 (e.g., Oligo-03 for EMX1 left-side rearrangement, Oligo-14 for EMX1 right-side rearrangement, Oligo-18 for CCR5 #1 left-side rearrangement, or Oligo-22 for CCR5 #1 right-side rearrangement) using SuperFi II Polymerase PCR Master Mix (ThermoFisher, Cat #12368010), with the following program: initial denaturing at 90-99° C. for 1-5 minute, 4-40 cycles of the 4-step denaturing-annealing-amplification (5-60 seconds at 90-99° C., 5-60 seconds at 75-92° C., 5-60 second at 55-75° C., 5-60 seconds at 67-75° C.), and final extension at 67-75° C. for 1-10 minutes, and hold at 4-12° C. PCR products are purified using column-based purification.

Next generation sequencing is then performed using the PCR product from the previous step, and sequencing is performed using an Illumina platform (e.g., Illumina MiSeq or NextSeq 2000) with optional >=0-95% PhiX (Illumina, Cat #FC-110-3001).

Paired-end sequencing reads from Illumina sequencing are merged using a paired-end reads merging software, e.g., PEAR (Zhang et al, Bioinformatics. 2014; 30:614-620), FLASH (Magoč and Salzberg, Bioinformatics. 2011; 27:2957-2963), BBMerge (Bushnell et al, PLoS One. 2017; 12:e0185056), or NGmerge (Gaspar, Bioinformatics. 2018; 19:536). The merged reads are then trimmed and filtered using software such as BBmap (https://jgi.doe.gov/data-and-tools/software-tools/bbtools/), samtools (Danecek et al, Gigascience. 2021; 10. doi:10.1093/gigascience/giab008), or custom scripts to remove Illumina adaptor sequences, low quality reads, reads containing the target flanking sequence on the side of the target site where the blocking oligonucleotide or cleavage reagent binds, and reads that do not contain the target flanking sequence on the side of the target site where the target-specific primer hybridizes. Selected reads are aligned to a human reference genome, e.g., GRCh37 (hg19), GRCh38 (hg38), or Telomere-to-Telomere assembly (e.g., T2T-CHM13v2.0), using an alignment software, e.g., Bowtie 2 (Langmead and Salzberg, Nat Methods. 2012; 9:357-359), BWA (Li and Durbin, Bioinformatics. 2009; 25:1754-1760), or Minimap2 (Li, Bioinformatics. 2018; 34:3094-3100). The aligned BAM file is converted into bed file using BEDTools (Quinlan, Bioinformatics. 2014; 47:11.12.1-34). For methods using UMIs, UMIs are collapsed to remove redundant sequencing reads using software such as Gencore (Chen et al, Bioinformatics. 2019; 20:606), UMI-tools (Smith et al, Genome Res. 2017; 27:491-499), UMI-Reducer (github.com/smangul1/UMI-Reducer), or custom scripts. Reads with candidate translocation break points within a suitable window flanking the target site (e.g., within 1, 3, 5, 10, 20, 40, 60, 80, 100, 200, or more bases) are identified and counted to quantify the number of rearrangements between the target site and other genomic loci. Statistical tests are then applied to establish a confidence score to each group of rearrangements at each distal rearranged locus (e.g., within a defined window of up 50, 100, 200, 500, 1000, 2000, 3000, or more bases at the distal rearranged locus). Visualization tools such as IGV (Thorvaldsdóttir et al, Brief Bioinform. 2013; 14:178-192) or Circos (Krzywinski et al, Genome Res. 2009; 19:1639-1645) are used to facilitate the identification and analysis of rearrangements at genomic scale.

### Example 8: Exemplary Method for Method II

The Exemplary oligonucleotides used are listed in Table 5.

Additional reagents included unloadedTn5 Transposase (Diagenode, C01070010, Diagenode), 2× Tagmentation buffer (Diagenode, C01019043), Platinum™ SuperFi II PCR Master Mix (ThermoFisher, Cat #12368010), dNTP Mix (10 mM each) (ThermoFisher, Cat #R0194), DNA purification kit: DNA Clean & Concentrator-5 (Zymo Research, Cat #D4004), AMPure XP Reagent (Beckman Coulter, Cat #A63880), Glycerol (MilliporeSigma, 356350500ML), 10% SDS (Fisher, BP2436200), 5M NaCl (Invitrogen, AM9760G), Tris HCl 1M, pH8.0 (Invitrogen, 15568-025), Genomic DNA (extracted from edited and unedited 293T cells), ddNTP, Dideoxynucleoside Triphosphate Set (MilliporeSigma, Cat #03732738001), NEBNext library Quant Kit for Illumina (NEB, E7630), Terminal transferase (TdT, NEB, M0315S), Alt-R™ S.p. HiFi Cas9 Nuclease V3 (IDT, 1081060), NEB buffer r3.1 (NEB, B6003S), Illumina NextSeq 1000/2000 P1 Reagents (300 Cycles) (Illumina, Cat #20050264), PhiX Control v3 (Illumina, Cat #FC-110-3001).

TABLE 16
Exemplary guide RNA sequences are shown in below.

| | Design | Sequence (PAM is underlined, | Coordinate in hg38 |
|---|---|---|---|
| Oligo ID | group | not included in sgRNA) | (chr strand start end) |
| Oligo-14 | CCR5_L | TGACATCAATTATTATACATCGG | chr3 + 46372932 46372954 |
| | | (SEQ ID NO: 33) | |
| Oligo-15 | CCR5_R | TTTTGCAGTTTATCAGGATGAGG | chr3 − 46373058 46373080 |
| | | (SEQ ID NO: 34) | |

In the first step, transposon-end containing oligos (e.g., Oligo-01A and Oligo-03A, or Oligo-02A and Oligo-03A) were resuspend in annealing buffer (40 mM Tris-HCl (pH8.0), 50 mM NaCl) to stock concentration of 100 μM. In a PCR tube, 10 μl oligo-01, 10 μl oligo-03 were mixed, vortexed and placed in PCR tubes in a thermocycler with the following program: 5 minutes at 95° C., cool to 65° C. (−0.1° C./second), 5 minutes at 65° C., cool to 25° C. (−0.1° C./second), 5 minutes at 25° C., and hold at 4° C.

Transposome complexes were assembled following the procedure below, as recommended by the manufacturer. The reagents were mixed in a PCR tube: 10 μL Tn5 transposase (2 ug/ul), 10 μL annealed adaptor. The reagents were mixed thoroughly and incubated at 23° C. for 30 minutes. 10 μl glycerol was added and mixed. The assembled transposome complex was stored at −20° C.

The workflow for this example is shown in FIG. 9. The tagmentation reaction was performed using genomic DNA extracted from gene edited samples or controls (using standard methods) using the following procedure: 100 ng genomic DNA from unedited control or edited sample was combined with 20 μL 2× Tagmentation buffer, 200 ng loaded Tn5 Transposase, and added H2O up to 40 μL. The reaction mixtures were mixed thoroughly and incubated at 55° C. for 15 minutes. 10 μL 0.2% SDS was added, and then Tn5 was inactivated for 10 min at 70° C. The tagmented DNA was purified using Zymo columns following the manufacturer's instructions and eluted in 21 μL.

Two separate ribonucleoprotein (RNP) assembly reactions and corresponding in vitro cleavage (IVC) reactions were then performed for each target, one reaction to detect genome rearrangements on the left-side of the target and the other reaction to detect genome rearrangements on the right-side of the target. CRISPR/Cas9 Nuclease, Alt-R™ S.p. HiFi Cas9 Nuclease V3 (IDT, 1081060), and CCR5 sgRNA (e.g., CCR5_L for left-side rearrangement, or CCR5_R right-side rearrangement) (Table 5A) were assembled into RNP with the following procedure: 32.5 μL H2O, 5 μL (10×) NEBuffer r3.1, 1.5 μL CCR5 sgRNA (10 μM), 0.8 μL HiFi Cas9 Nuclease (diluted to 6.2 μM). The RNP reactions were mixed thoroughly and incubated at room temperature for 10 minutes.

Tagmented DNA was cleaved in vitro using Cas9 RNP assembled in the previous step. 10 μL DNA was added into the RNP solution and incubated at 37° C. for 2 hours. The products were purified using Zymo columns following manufacture's instructions and eluted in 11 ul.

The 3′ ends of DNA molecules were then blocked using ddNTP and terminal transferase (TdT, NEB, M0315S) using following procedure: 10 μL tagmented DNA, 5 μL 10× TdT buffer, 2 μL (40U) TdT, 5 μL CoCl2 solution, 1 μL ddNTP (10 mM), and add up to 50 μL. The reactions were incubated at 37° C. for 2 hours, 70° C. for 10 min, and cooled down to 4° C. The DNA was then purified using Zymo column following the manufacturer's instructions and eluted in 11 μL.

PCR using blocked IVC DNA from the previous step was then performed with a common primer annealed to the adapter region, and a target specific primer. PCR was performed using 4-step PCR cycling protocol with tagmented DNA, Primer 1 (Oligo-04A, common to all targets and rearrangement of both sides), Primer 2 (e.g., Oligo-06A for CCR5 #1 left-side rearrangement, or Oligo-10A for CCR5 #1 right-side rearrangement), using SuperFi II Polymerase PCR Master Mix (ThermoFisher, Cat #12368010). PCR mixture was prepared as follows: 25 μL2× PCR Master Mix, 10 μL Tagmented DNA, 1.25 μL Primer 1 (10 μM), 2.5 μL Primer 2 (10 μM), 10 μL Blocker 0 (10 μM), and add H2O up to 50 μL. PCR was performed using the following program: initial denaturing at 98° C. for 30 seconds, 15 cycles of the 4-step denaturing-annealing-amplification (10 seconds at 98° C., 10 seconds at 78° C., 10 second at 60° C., 60 seconds at 72° C.), and final extension at 72° C. for 5 minutes, and hold at 4° C. PCR products were purified using AMpure XP beads (1×) and eluted in 12 μL.

Nested PCR was then performed with a barcode tag. PCR was performed with the PCR products from the previous step, Primer 3 (Oligo-04A, common to all targets and rearrangement of both sides), Primer 4 (e.g., Oligo-20 for CCR5 #1 left-side rearrangement, or Oligo-24 for CCR5 #1 right-side rearrangement) using SuperFi II Polymerase PCR Master Mix (ThermoFisher, Cat #12368010). PCR mixtures were prepared as follows: 25 μL 2× PCR Master Mix, 10 μL PCR product, 1.25 μL Primer 3 (10 μM), 2.5 L Primer 4 (10 μM), 10 μL Blocker 0 (10 μM), and add H2O up to 50 μL. PCR was performed using the following program: initial denaturing at 98° C. for 30 seconds, 15 cycles of the 4-step denaturing-annealing-amplification (10 seconds at 98° C., 10 seconds at 78° C., 10 second at 60° C., seconds at 72° C.), and final extension at 72° C. for 5 minutes, and hold at 4° C. PCR products were purified using AMpure XP beads, and eluted in 12 μL.

Tag PCR was then performed with sequencing primers. PCR was performed using the PCR products from the previous step, Primer 1 (Oligo-04A, common to all targets and rearrangement of both sides), (e.g., any Oligo-13A, or Illumina indexing primer from New England Biolabs, NEB #E7611A) and SuperFi II Polymerase PCR Master Mix (ThermoFisher, Cat #12368010). PCR mixtures were prepared as follows: 25 μL 2× PCR Master Mix, 10 μL PCR product, 5 μL Primer 5 (10 μM), 5 μL Primer 6 (10 μM), 5 μL Blocker 0 (20 μM). PCR was performed using the following program: initial denaturing at 98° C. for 30 seconds, 15 cycles of the 4-step denaturing-annealing-amplification (10 seconds at 98° C., 10 seconds at 78° C., 10 second at 60° C., seconds at 72° C.), and final extension at 72° C. for 5 minutes, and hold at 4° C. PCR products were purified using AMpure XP beads, and eluted in 52 μL.

50 μL of the supernatant was transferred to a new tube for library quantification using the NEBNext library Quant Kit for Illumina (NEB, E7630) following the manufacture's instructions. Libraries were normalized and pooled for sequencing.

Next generation sequencing was then performed using the PCR product from the previous step, and sequencing was performed using an Illumina platform (NextSeq 2000) with 20% PhiX (Illumina, Cat #FC-110-3001).

Paired-end sequencing reads from Illumina sequencing were processed using a customized NGS analysis pipeline. The sequence reads were first checked for overall quality using FastQC followed by consolidating FastQC results using MultiQC. The reads were then subjected to Adapter and primer sequence trimming using Cutadapt (DOI:10.14806/ej.17.1.200) followed by trimming low quality bases using Trimmomatic (Bolger et al, Bioinformatics. 2014; 30 (15): 2114-20). The trimming in the reads was verified by running FastQC and MultQC steps on trimmed reads fastq files to ascertain the removal of adaptors. The resulting reads were aligned with human genome reference sequence (hg38) using BWA-MEM (Li and Durbin, Bioinformatics, 2009; 25:1754-1760) tool with default parameters. The generated Sequence Alignment Map (SAM) file was sorted by coordinate and converted to Binary Alignment Map (BAM) file using PicardTools (http://broadinstitute.github.io/picard). The BAM file was then filtered to remove low-quality reads and to keep the reads with quality score ≥30 using Samtools. The resulting high quality BAM file was then checked using the CollectInsertSizeMatrix and CollectAlignemntSummary Matrics modules from PicardTools followed by FastQC and MultiQC report generation. The reads in the sorted high quality read BAM were grouped based on UMIs using UMI-Tools with paired option and saved as grouped BAM followed by BAM indexing using Samtools. The grouped BAM file was then de-duplicated using UMI-Tools with paired option to remove PCR duplicate reads. The FGSV tool was then used to discover the structural variation pileup by searching for split read mapping and read pairs that map across breakpoints in the BAM file using the FGSV SVPileup module. The AggregateSvPileup module of FGSV then aggregated information across nearby pileups to call structural variants. Only the pileups containing at least one breakpoint on the target were considered as real hits, and as high confidence hits with at least 10 split reads. IGV (Thorvaldsdóttir et al, Brief Bioinform. 2013; 14:178-192) was used to facilitate the identification and analysis of rearrangements at genomic scale.

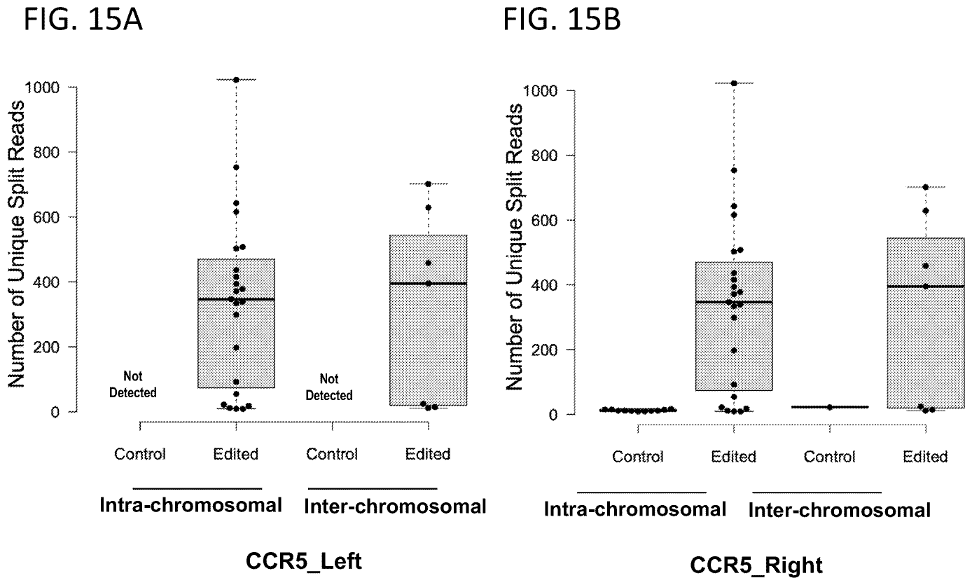

DNA rearrangements detected by Example 8 in control and CCR5 edited cells are shown in Table 17 below. The criteria used to filter out noise is the same with Method I_{A}. As shown in Table 17, 27 and 30 hits of high confidence passed the filters in edited cells, for the left-side and right-side DNA rearrangement events, respectively, while only 0 and 12 hits were captured in control cells for the left-side events, and right-side events, respectively. However, besides more hits, the numbers of the split reads to support these hits were also much higher in edited cells in FIG. 15.

TABLE 17
Summary of DNA rearrangement by Example
8 in control and CCR5 edited cells.

| Samples | Side | Intra Chromosomal | Inter Chromosomal |
|---|---|---|---|
| Control | Left | 0 | 0 |
| CCR5 edited | | 20 | 7 |
| Control | Right | 11 | 1 |
| CCR5 edited | | 23 | 7 |

Note:
DNA rearrangment event with split read number <10 were filtered out. One breakpoint must be located at ON target cutting site +/−25 bp.

Comparison of split reads between control and CCR5 edited cells shown in FIG. 15.

DNA rearrangements identified by Example 8 in CCR5 edited cells are displayed in Table 18 below. The genomic coordinates of the DNA breakpoints between DNA rearrangements, number of unique split-reads based on UMI-tools, extracted reads to show the split reads, and note to further explain the potential mechanism for this DNA rearrangements were included in the table. It is worth noting that the DNA rearrangements between CCR5 and its homologous gene CCR2, inter-chromosomal translocation between CCR5 on chromosome 3 and RNF17/CENPJ on chromosome 13 were also identified by CAST-seq (Turchiano et al, 2021). The presence of fused DNA of chr3/chr13 and acentric chromosome were also validated using PCR (FIGS. 13A-B). Therefore, SAFER detection Method II can efficiently capture intra- and inter-chromosomal rearrangements.

TABLE 18
Summary of DNA rearrangement by Example 8 in CCR5 edited cells.

| | | | | Unique | | |
|---|---|---|---|---|---|---|
| Cat | | On-target | Non-target | Split_ | | |
| egory | Side | breakpoint | breakpoint | reads | Exemplar read | Note |
| Intra | Left | chr3: | chr3: | 1023 | Read name: | The event represents a |
| chromo | | 46373001- | 46372545- | | VH01033: 21: | large deletion caused |
| somal | | 46373007 | 46372561 | | AACCM73M5: 1: 1303: | by CRISPR/Cas9 editing. |
| re- | | | | | 41825:48898:ITGAT | No off-target site was |
| arrange | | | | | GGCGGG | predicted at the non- |
| ment | | | | | Sequence: | target breakpoint site. |
| | | | | | CCTGGGCGACAGAGTGA | Gene: CCR5 |
| | | | | | GACCCTGTCTCACAACA | |
| | | | | | ACAACAACAACAACTCC | |
| | | | | | GCTCTACTCACTGGTGTT | |
| | | | | | CATCTTTGGTTTTGTGGG | |
| | | | | | CAACATGCTGGTCATCC | |
| | | | | | TCATCCTGATAAACTGC | |
| | | | | | AAAAGGCTGAAGAGC | |
| | | | | | (SEQ ID NO: 83) | |
| | | | | | Alignments: | |
| | | | | | chr3: | |
| | | | | | 46,373,003- | |
| | | | | | 46,373,091 (+) | |
| | | | | | chr3: | |
| | | | | | 46,372,500- | |
| | | | | | 46,372,548 | |
| | | | | | (+) underlined | |
| | Left | chr3: | chr3: | 372 | Read name: | This event represents a |
| | | 46373003- | 46486211- | | VH01033: 21: | an intergenic inversion |
| | | 46373003 | 46486214 | | AACCM73M5: 1: 1202: | that is caused by off- |
| | | | | | 27775: 37879: | target editing or |
| | | | | | rACGGCATCCT | homology mediated |
| | | | | | Sequence: | recombination. |
| | | | | | ATGAGGATGACCAGCAT | Predicted off-target |
| | | | | | GTTGCCCACAAAACCAA | site: |
| | | | | | AGATGAACACCAGTGAG | CACCAGCGAGTAG |
| | | | | | TAGAGCGGAGAGCAGA | AGCGGAGGCAGGA |
| | | | | | CAAACAGACAAACAAA | G (SEQ ID NO: 61) |
| | | | | | CAAAACACCTGAAGCCC | chr3: 46357657- |
| | | | | | CTGACTAACAATCAATA | 46357683 and |
| | | | | | GGCAACATCCAGGAAGA | homology sequence in |
| | | | | | TTGTAACCCCATAGTAC | CCR2 and CCR5: |
| | | | | | (SEQ ID NO: 84) | CTGGTGTTCATCTT |
| | | | | | Alignments: | TGGTTTTGTGGGCA |
| | | | | | chr3: | ACATGCTGGTC |
| | | | | | 46,373,003- | (SEQ ID NO: 62) |
| | | | | | 46,373,063 (−) | Gene: CCR2, CCR5 |
| | | | | | chr3: | |
| | | | | | 46,486.210- | |
| | | | | | 46,486.295 | |
| | | | | | (+) underlined | |
| | Right | chr3: | chr3: | 6566 | Read name: | The event represents a |
| | | 46372990- | 46373945- | | VH01033: 30: | large deletion caused |
| | | 46373002 | 46373956 | | AAF5TJWM5: | by CRISPR/Cas9 editing. |
| | | | | | 1: 2203: Sequence: | No off-target site was |
| | | | | | 57636: 20822: | predicted at the non- |
| | | | | | rACCTCGGAAA | target breakpoint site. |
| | | | | | TACATCGGAGCCCTGCC | Gene: CCR5 |
| | | | | | AAAAAATCAATGTGAAG | |
| | | | | | CAAATCGCAGCCCGCCT | |
| | | | | | CTGTGGGCTTGTGACAC | |
| | | | | | GGACTCAAGTGGGCTGG | |
| | | | | | TGACCCAGTCAGAGTTG | |
| | | | | | TGCACATGGCTTAGTTTT | |
| | | | | | CATACACAGCCTGGGCT | |
| | | | | | GGGG (SEQ ID NO: 85) | |
| | | | | | Alignments: | |
| | | | | | chr3: | |
| | | | | | 46,372,946-46,372,995 | |
| | | | | | (+) | |
| | | | | | chr3: | |
| | | | | | 46,373,946-46,374,037 | |
| | | | | | (+) underlined | |
| | Right | chr3: | chr3: | 10651 | Read name: | This event represents |
| | | 46372978- | 46372540- | | VH01033: 30: | an intergenic inversion |
| | | 46373001 | 46372654 | | AAF5TJWM5: 1: 1101: | that is caused by off- |
| | | | | | 32982: 28773: | target editing or |
| | | | | | rCTAATCAAAC | homology mediated |
| | | | | | Sequence: | recombination. |
| | | | | | AGCCTGGGCGACAGAGT | Predicted off-target |
| | | | | | GAGACCCTGTCTCACAA | site: |
| | | | | | CAACAACAACAACAACA | CACCAGCGAGTAG |
| | | | | | AAAAGGCTGAGCTGCAC | AGCGGAGGCAGGA |
| | | | | | CATGCTTGACCCAGTTT | G (SEQ ID NO: 61) |
| | | | | | CTTAACTGGAGGCGGGC | chr3:46357657- |
| | | | | | TGCGATTTGCTTCACATT | 46357683 and |
| | | | | | GATTTTTTGGCAGGGCT | homology sequence in |
| | | | | | CCGATGTA (SEQ ID NO: | CCR2 and CCR5: |
| | | | | | 86) | CTGGTGTTCATCTT |
| | | | | | Alignments: | TGGTTTTGTGGGCA |
| | | | | | chr3: | ACATGCTGGTC |
| | | | | | 46,372,498-46,372,588 | (SEQ ID NO: 62) |
| | | | | | (+) underlined | Gene: CCR2, CCR5 |
| | | | | | chr3: | |
| | | | | | 46,372,946-46,373,000 | |
| | | | | | (−) | |
| | right | chr3: | chr3: | 3805 | Read name: | This event represents an |
| | | 46372986- | 46357615- | | VH01033: 30: | intergenic inversion |
| | | 46373001 | 46357635 | | AAF5TJWM5: 1: 2104: | that is caused by off- |
| | | | | | 20844: 22204: | target editing or |
| | | | | | rGAACATGCTT | homology mediated |
| | | | | | Sequence: | recombination. |
| | | | | | TACATCGGAGCCCTGCC | Predicted off-target |
| | | | | | AAAAAATCAATGTGAAG | site: |
| | | | | | CAAATCGCAGCCCGCCT | CACCAGCGAGTAG |
| | | | | | CCCTGTCATAAATTTGA | AGCGGAGGCAGGA |
| | | | | | CGTGAAGCAAATTGGGG | G (SEQ ID NO: 61) |
| | | | | | CCCAACTCCTGCC (SEQ | chr3:46357657- |
| | | | | | ID NO: 87) | 46357683 and |
| | | | | | Alignments: | homology sequence in |
| | | | | | chr3: | CCR2 and CCR5: |
| | | | | | 46,372,946- | CTGGTGTTCATCTT |
| | | | | | 46,372,999 (+) | TGGTTTTGTGGGCA |
| | | | | | chr3: | ACATGCTGGTC |
| | | | | | 46,357,619-46,357,664 | (SEQ ID NO: 62) |
| | | | | | (+) underlined | Gene: CCR2, CCR5 |
| Inter | Left | chr3: | chr13: | 459 | Read name: | These events represent |
| chromo | | 46372999- | 24886337- | | VH01033: 21: | interchromosomal |
| somal | | 46373012 | 24886353 | | AACCM73M5: 1: 1303: | translocation that is |
| re- | | | | | 21394: 11999: rGCGG | mediated by Off-target |
| arrang | | | | | CGGACG | editing. |
| ement | | | | | Sequence: | On-target site: |
| | | | | | CAGCACAGCCGCCTCAC | chr3:46372995- |
| | | | | | ACGCCCAGCAGCTTGTT | 46373017 (−) |
| | | | | | GGCTGACCTCCGCTCTA | GTGAGTAGAGCGG |
| | | | | | CTCACTGGTGTTCATCTT | AGGCAGGAGG (SEQ |
| | | | | | TGGTTTTGTGGGCAACA | ID NO: 30) |
| | | | | | TGCTGGTCATCCTCATCC | OFF-target site: |
| | | | | | TGATAAACTGCAAAAGG | G()GAGGAGAGCGG |
| | | | | | CTGAAGAGC (SEQ ID | AGGCAGGTGG |
| | | | | | NO: 88) | Chr13: 24886320- |
| | | | | | Alignments: | 24886342 (+) |
| | | | | | chr3: | Gene: CCR5 and |
| | | | | | 46,373,001- | RNF17/CENPJ |
| | | | | | 46,373,090 (+) | |
| | | | | | chr13: | |
| | | | | | 24,886,342-24,886382 | |
| | | | | | (−) underlined | |
| | Right | chr3: | chr13: | 127 | Read name: | |
| | | 24637995- | 24886335- | | VH01033: 30: | |
| | | 46373001 | 24886337 | | AAF5TJWM5: 1: 2303: | |
| | | | | | 30350: 25763: | |
| | | | | | rCTCCCGCCCC | |
| | | | | | Sequence: | |
| | | | | | TACATCGGAGCCCTGCC | |
| | | | | | AAAAAATCAATGTGAAG | |
| | | | | | CAAATCGCAGCCCGCAG | |
| | | | | | GTGGTCAGCCAACAAGC | |
| | | | | | TGCTGGGCGTGTGAGGC | |
| | | | | | GGCTGTGCTGTGCCTGT | |
| | | | | | GAGACAGGAATGGTTCC | |
| | | | | | ATTACTC (SEQ ID | |
| | | | | | NO: 89) | |
| | | | | | Alignments: | |
| | | | | | chr3: | |
| | | | | | 46,372,946- | |
| | | | | | 46,372,995 (+) | |
| | | | | | chr13: | |
| | | | | | 24.886.336-24.886.413 | |
| | | | | | (+) underlined | |

### Example 9: Exemplary Method for Method III

Exemplary oligonucleotides are listed in Table 1.

Additional reagents include Tn5 transposase, e.g., Robust Tn5 Transposase (Cat #EMQZ1422, Creative Biogene), DNA polymerase PCR master mix, e.g., Platinum™ SuperFi II PCR Master Mix (ThermoFisher, Cat #12368010), DNA polymerase with PCR buffer, e.g., Q5 High-Fidelity DNA Polymerase (New England Biolabs, Cat #M0491S), dNTP, e.g., dNTP Mix (10 mM each) (ThermoFisher, Cat #R0194), DNA purification kit: Column kit, e.g., DNA Clean & Concentrator-5 (Zymo Research, Cat #D4004), Magnetic beads, e.g., AMPure XP Reagent (Beckman, Cat #A63880), RNA purification kit, e.g., RNA Clean & Concentrator-5 (Zymo Research, Cat #R1013), Illumina sequencing kit, e.g., NextSeq 1000/2000 P1 Reagents (300 Cycles) (Cat #20050264), MiSeq Reagent Kit v3 (150-cycle) (Cat #MS-102-3001), MiSeq Reagent Kit v2 (300-cycles) (Cat #MS-102-2002), PhiX Control v3 (Illumina, Cat #FC-110-3001), Strand displacement polymerase, e.g., phi29 DNA Polymerase (New England Biolabs, Cat #M0269S), or Bst 3.0 DNA Polymerase (New England Biolabs, Cat #M0374S), DNase I (RNase-free) (New England Biolabs, Cat #M0303S), RNase H, e.g., (New England Biolabs, Cat #M0297S), In vitro transcription kit, e.g., MAXIscript™ T7 Transcription Kit (ThermoFisher, Cat #AM1312), Reverse transcription kit, e.g., SuperScript IV First-Strand Synthesis System (ThermoFisher, Cat #18091050), STE buffer (10 mM Tris-HCl (pH 8.0), 1 mM EDTA, 0.1M NaCl), Magnesium Chloride (MgCl₂) Solution (New England Biolabs, Cat #B9021S).

In the first step, Tn5 transposomes are assembled. Transposon-end containing oligos (e.g., Oligo-01 and Oligo-02 for non-UMI version, or Oligo-26 and Oligo-02 for UMI version) are annealed in STE buffer with the following program: 1-10 minutes at 90-99° C., 40-500 cycles of 1 minutes at 90-99° C. (decrease temperature 0.2-2° C. every cycle until reaching 1-12° C.). Transposome complexes are assembled in TPS buffer following the procedure below, as recommended by the manufacturer (Creative Biogene, Cat #EMQZ1422). The reagents are mixed thoroughly and incubated at 15-40° C. for 5-120 minutes.

| Component | Volume | |
|---|---|---|
| Tn5 transposase | 1-10 | μL |
| Adaptor | 0.5-8 | μL |
| 10x TPS buffer | 2 | μL |

| | |
|---|---|
| Sterile water | add to 20 μL |

The workflow for this example is shown in FIG. 5. The tagmentation reaction is performed in which genomic DNA extracted from gene edited samples or controls (using standard methods) is tagmented with the assembled transposome complex following procedure. An exemplary reaction mixture is shown below:

| Component | Volume | |
|---|---|---|
| Genomic DNA | varies (5-500 ng) | |
| 5x LM buffer | 6 | μL |
| Transposome | 0.1-8 | μL |

| | |
|---|---|
| Sterile water | add to 30 μL |

The reaction components are mixed thoroughly and incubated at 30-65° C. for 5-120 minutes. Transposase is then removed by 60-80° C. for 5-120 minutes in the presence of 0.1-10 mM EDTA. Both ends of the transposed fragments is filled and extended by Q5 High-Fidelity DNA Polymerase (New England Biolabs, Cat #M0491S) at 60-75° C. for 5-600 seconds in the presence of 0.2-4 mM MgCl₂ and 20-1000 μM dNTPs, without the supplement of specific Q5 reaction buffer. Products are purified using column-based purification.

In vitro transcription is then performed using MAXIscript™ T7 Transcription Kit (ThermoFisher, Cat #AM1312) and the tagmented DNA from the previous step, by incubating at 25-45° C. for 1-600 min. The next step is performed immediately. TURBO DNase from the MAXIscript™ T7 Transcription Kit (ThermoFisher, Cat #AM1312) is added to the previous reaction mixture and incubated at 20-45° C. for 1-240 min. RNA product is purified using an RNA purification kit, e.g., RNA Clean & Concentrator-5 (Zymo Research, Cat #R1013).

DNA probe hybridization and following RNase H treatment are then performed in two separate reactions for each target, one reaction for detecting genome rearrangement on the left-side of the target and the other reaction for detecting genome rearrangement on the right-side of the target. Probe 0 (e.g., Oligo-13 for EMX1 left-side rearrangement, Oligo-17 for EMX1 right-side rearrangement, Oligo-21 for CCR5 #1 left-side rearrangement, or Oligo-25 for CCR5 #1 right-side rearrangement)) is added to RNA products purified from the previous step with RNase H Reaction Buffer. Mixture is denatured at 85-99° C. for 1-10 minutes and cool down to 4-28° C. RNase H is added to the reaction and incubated at 16-45° C. for 1-300 minutes. RNase H is then inactivated at 60-90° C. for 5-240 minutes. Product is purified using an RNA purification kit, e.g., RNA Clean & Concentrator-5 (Zymo Research, Cat #R1013).

Reverse transcription is then performed. Briefly, RNase H treated RNA is reverse transcribed to DNA using target specific Primer 2 (e.g., Oligo-05 for EMX1 left-side rearrangement, Oligo-15 for EMX1 right-side rearrangement, Oligo-19 for CCR5 #1 left-side rearrangement, or Oligo-23 for CCR5 #1 right-side rearrangement) and SuperScript IV First-Strand Synthesis System (ThermoFisher, Cat #18091050). First, RNA, dNTPs and primer are mixed and heated at 65° C. for 1-20 minutes, then incubated at 1-12° C. for 10-600 seconds for primer annealing. Next, 5× SSIV buffer, DTT, Ribonuclease inhibitor and SuperScript IV Reverse Transcriptase are added to the mixture, and the combined reaction mixture is incubated at 40-60° C. for 2-120 minutes. The reaction is inactivated by incubating it at 60-90° C. for 5-240 minutes. To remove RNA, 0.1-10 μL of RNase H is added to the reaction mixture, mixed, and incubated at 20-55° C. for 5-120 minutes. Product is purified using column-based purification.

The first-strand cDNA synthesized from the previous step is used in the first PCR with common Primer 1 (Oligo-04, common to all targets and rearrangement of both sides), target specific Primer 2 (e.g., Oligo-05 for EMX1 left-side rearrangement, Oligo-15 for EMX1 right-side rearrangement, Oligo-19 for CCR5 #1 left-side rearrangement, or Oligo-23 for CCR5 #1 right-side rearrangement), and SuperFi II Polymerase PCR Master Mix (ThermoFisher, Cat #12368010), with the following program: initial denaturing at 90-99° C. for 1-5 minute, 20-40 cycles of the 3-step denaturing-annealing-amplification (5-60 seconds at 90-99° C., 5-60 second at 55-75° C., 5-60 seconds at 67-75° C.), and final extension at 67-75° C. for 1-10 minutes, and hold at 4-12° C. PCR products are purified using column-based purification.

Nested PCR is then performed with barcode tags. PCR is performed with PCR product from the previous step, Primer 3 (Oligo-06, common to all targets and rearrangement of both sides) and Primer 4 (e.g., Oligo-07 for EMX1 left-side rearrangement, Oligo-16 for EMX1 right-side rearrangement, Oligo-20 for CCR5 #1 left-side rearrangement, or Oligo-24 for CCR5 #1 right-side rearrangement) using SuperFi II Polymerase PCR Master Mix (ThermoFisher, Cat #12368010), with the following program: initial denaturing at 90-99° C. for 1-5 minute, 4-40 cycles of the 3-step denaturing-annealing-amplification (5-60 seconds at 90-99° C., 5-60 second at 55-75° C., 5-60 seconds at 67-75° C.), and final extension at 67-75° C. for 1-10 minutes, and hold at 4-12° C. PCR products are purified using column-based purification.

Tag PCR is then performed with sequencing primers. PCR is performed with PCR product from the previous step, Primer 5 (e.g., Oligo-08, an example of Illumina indexing primer from New England Bio Labs, NEB #E7603A), and Primer 6 (e.g., Oligo-09, an example of Illumina indexing primer from New England Bio Labs, NEB #E7611A) using SuperFi II Polymerase PCR Master Mix (ThermoFisher, Cat #12368010), with the following program: initial denaturing at 90-99° C. for 1-5 minute, 4-40 cycles of the 3-step denaturing-annealing-amplification (5-60 seconds at 90-99° C., 5-60 second at 55-75° C., 5-60 seconds at 67-75° C.), and final extension at 67-75° C. for 1-10 minutes, and hold at 4-12° C. PCR products are purified using column-based purification.

Next generation sequencing is then performed using the PCR product from the previous step, and sequencing is performed using an Illumina platform (e.g., Illumina MiSeq or NextSeq 2000) with optional >=0-95% PhiX (Illumina, Cat #FC-110-3001).

Paired-end sequencing reads from Illumina sequencing are merged using a paired-end reads merging software, e.g., PEAR (Zhang et al, Bioinformatics. 2014; 30:614-620), FLASH (Magoč and Salzberg, Bioinformatics. 2011; 27:2957-2963), BBMerge (Bushnell et al, PLoS One. 2017; 12: e0185056), or NGmerge (Gaspar, Bioinformatics. 2018; 19:536). The merged reads are then trimmed and filtered using software such as BBmap (https://jgi.doe.gov/data-and-tools/software-tools/bbtools/), samtools (Danecek et al, Gigascience. 2021; 10. doi: 10.1093/gigascience/giab008), or custom scripts to remove Illumina adaptor sequences, low quality reads, reads containing the target flanking sequence on the side of the target site where the blocking oligonucleotide or cleavage reagent binds, and reads that do not contain the target flanking sequence on the side of the target site where the target-specific primer hybridizes. Selected reads are aligned to a human reference genome, e.g., GRCh37 (hg19), GRCh38 (hg38), or Telomere-to-Telomere assembly (e.g., T2T-CHM13v2.0), using an alignment software, e.g., Bowtie 2 (Langmead and Salzberg, Nat Methods. 2012; 9:357-359), BWA (Li and Durbin, Bioinformatics. 2009; 25:1754-1760), or Minimap2 (Li, Bioinformatics. 2018; 34:3094-3100). The aligned BAM file is converted into bed file using BEDTools (Quinlan, Bioinformatics. 2014; 47:11.12.1-34). For methods using UMIs, UMIs are collapsed to remove redundant sequencing reads using software such as Gencore (Chen et al, Bioinformatics. 2019; 20:606), UMI-tools (Smith et al, Genome Res. 2017; 27:491-499), UMI-Reducer (github.com/smangul1/UMI-Reducer), or custom scripts.

Reads with candidate translocation break points within a suitable window flanking the target site (e.g., within 1, 3, 5, 10, 20, 40, 60, 80, 100, 200, or more bases) are identified and counted to quantify the number of rearrangements between the target site and other genomic loci. Statistical tests are then applied to establish a confidence score to each group of rearrangements at each distal rearranged locus (e.g., within a defined window of up 50, 100, 200, 500, 1000, 2000, 3000, or more bases at the distal rearranged locus). Visualization tools such as IGV (Thorvaldsdóttir et al, Brief Bioinform. 2013; 14:178-192) or Circos (Krzywinski et al, Genome Res. 2009; 19:1639-1645) are used to facilitate the identification and analysis of rearrangements at genomic scale.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

INFORMAL SEQUENCE LISTING

| | | SEQ ID |
|---|---|---|
| Name | Sequence | NO |
| Tn5 adapter top | CGTACCGATACGCTCGGACAGATGTGTATAAGAGAC | 1 |
| | AG | |
| Tn5 adapter bottom | CTGTCTCTTATACACATCT | 2 |
| Blocker 0- | +T+C+CTCCAG+CTTCT+GCCGT+T+T+G/3AmMO | 3 |
| EMX1_L | | |
| Primer 1 | GCGTACCGATACGCTCGGAC | 4 |
| Primer 2 | GGTTTGTGGTTGCCCACCCT | 5 |
| Primer 3 | ACACTCTTTCCCTACACGACGCTCTTCCGATCTCGTAC | 6 |
| | CGATACGCTCGGAC | |
| Primer 4 | GTGACTGGAGTTCAGACGTGTGCTCTTCCGATCTCCT | 7 |
| | CCCCATTGGCCTGCTTC | |
| Primer 5 | AATGATACGGCGACCACCGAGATCTACACTATAGCCTA | 8 |
| | CACTCTTTCCCTACACGACGCTCTTCCGATC*T | |
| Primer 6 | CAAGCAGAAGACGGCATACGAGATCGAGTAATGTGAC | 9 |
| | TGGAGTTCAGACGTGTGCTCTTCCGATC*T | |
| Tn5 adapter top U | CGTACCGAUACGCTCGGACUAGATGTGTATAAGAGA | 10 |
| | CAG | |
| Tn5 adapter bottom | CTGTCTCTTATACACATCT/3ddC/ | 11 |
| 3′ blocked | | |
| Tn5 adapter top | TAATACGACTCACTATAGCGTACCGATACGCTCGGAC | 12 |
| T7p | AGATGTGTATAAGAGACAG | |
| Probe 0-EMX1_L | TCCTCCAGCTTCTGCCGTTTG | 13 |
| Blocker 0- | +C+G+AAGCA+GGCCA+ATGGG+G+A+G/3AmMO | 14 |
| EMX1_R | | |
| Primer 2- | TCTCATCTGTGCCCCTCCCT | 15 |
| EMX1_R | | |
| Primer 4- | GTGACTGGAGTTCAGACGTGTGCTCTTCCGATCTGTG | 16 |
| EMX1_R | TGGTTCCAGAACCGGAGG | |
| Probe 0-EMX1_R | CGAAGCAGGCCAATGGGGAG | 17 |
| Blocker 0- | +A+G+GGCTCC+GATGTA+TAATAA+T+T+G/3AmMO/ | 18 |
| CCR5#1_L | | |
| Primer 2- | AGGTAGATGTCAGTCATGCT | 19 |
| CCR5#1_L | | |
| Primer 4- | GTGACTGGAGTTCAGACGTGTGCTCTTCCGATCTGCT | 20 |
| CCR5#1_L | CTTCAGCCTTTTGCAGTTTATCAG | |
| Probe 0- | AGGGCTCCGATGTATAATAATTG | 21 |
| CCR5#1_L | | |
| Blocker 0- | +G+G+CAACAT+GCTGGT+CATCC+T+C+A/3AmMO/ | 22 |
| CCR5#1_R | | |
| Primer 2- | ACAGGGTGGAACAAGATGGAT | 23 |
| CCR5#1_R | | |
| Primer 4- | GTGACTGGAGTTCAGACGTGTGCTCTTCCGATCTTAC | 24 |
| CCR5#1_R | ATCGGAGCCCTGCCAAA | |
| Probe 0- | GGCAACATGCTGGTCATCCTCA | 25 |
| CCR5#1_R | | |
| Tn5 adapter top | CGTACCGATACGCTCGGACNNNNNNNNNNNNAGATG | 26 |
| UMI | TGTATAAGAGACAG | |
| Tn5 adapter top U | CGTACCGAUACGCTCGGACUNNNNNNNNNNNNUAG | 27 |
| UMI | ATGTGTATAAGAGACAG | |
| Tn5 adapter top | TAATACGACTCACTATAGCGTACCGATACGCTCGGAC | 28 |
| T7p UMI | NNNNNNNNNNNNAGATGTGTATAAGAGACAG | |
| EMX1 target | GAGTCCGAGCAGAAGAAGAAGGG | 29 |
| sequence | | |
| CCR5#1 target | GTGAGTAGAGCGGAGGCAGGAGG | 30 |
| sequence | | |
| EMX1_L | CAAACGGCAGAAGCTGGAGGAGG | 31 |
| EMX1_R | CTCCCCATTGGCCTGCTTCGTGG | 32 |
| CCR5_L | TGACATCAATTATTATACATCGG | 33 |
| CCR5_R | TTTTGCAGTTTATCAGGATGAGG | 34 |
| i5_N501_UMI_Tn5- | AATGATACGGCGACCACCGAGATCTACACTAGATCG | 35 |
| A | CNNNNNNNNNNTCGTCGGCAGCGTCAGATGTGTATA | |
| | AGAGACAG | |
| 15_N502_UMI_Tn5- | AATGATACGGCGACCACCGAGATCTACACCTCTCTAT | 36 |
| A | NNNNNNNNNNTCGTCGGCAGCGTCAGATGTGTATAA | |
| | GAGACAG | |
| i5_N503_UMI_Tn5- | AATGATACGGCGACCACCGAGATCTACACTATCCTCT | 37 |
| A | NNNNNNNNNNTCGTCGGCAGCGTCAGATGTGTATAA | |
| | GAGACAG | |
| 15_N504_UMI_Tn5- | AATGATACGGCGACCACCGAGATCTACACAGAGTAG | 38 |
| A | ANNNNNNNNNNTCGTCGGCAGCGTCAGATGTGTATA | |
| | AGAGACAG | |
| 15_N505_UMI_Tn5- | AATGATACGGCGACCACCGAGATCTACACGTAAGGA | 39 |
| A | GNNNNNNNNNNTCGTCGGCAGCGTCAGATGTGTATA | |
| | AGAGACAG | |
| 15_N506_UMI_Tn5- | AATGATACGGCGACCACCGAGATCTACACACTGCAT | 40 |
| A | ANNNNNNNNNNTCGTCGGCAGCGTCAGATGTGTATA | |
| | AGAGACAG | |
| 15_N507_UMI_Tn5- | AATGATACGGCGACCACCGAGATCTACACAAGGAGT | 41 |
| A | ANNNNNNNNNNTCGTCGGCAGCGTCAGATGTGTATA | |
| | AGAGACAG | |
| 15_N508_UMI_Tn5- | AATGATACGGCGACCACCGAGATCTACACCTAAGCC | 42 |
| A | TNNNNNNNNNNTCGTCGGCAGCGTCAGATGTGTATA | |
| | AGAGACAG | |
| 15_N501_UMI_Tn5- | AATGATACGGCGACCACCGAGATC/ideoxy U/ACACTA | 43 |
| A_Uracil | GATCGCNNNNNNNNNN/ideoxyU/CGTCGGCAGCGTCA | |
| | GATGTGTATAAGAGACAG | |
| Tn5-B 3′blocked | CTGTCTCTTATACACATCT/3ddC/ | 44 |
| Primer 1/3/5 | AATGATACGGCGACCACCGAGATCTACAC | 45 |
| Blocker 0- | +A+GG+GC+TC+CG+AT+GT+AT+AA+TA+AT+T+G/ | 46 |
| CCR5#1_L | 3AmMO/ | |
| Blocker 0- | +T+GG+GC+AA+CA+TG+CT+GG+TC+AT+CC+T+C/ | 47 |
| CCR5#1_R | 3AmMO/ | |
| Primer 6_N701 | CAAGCAGAAGACGGCATACGAGATAGCGGAATGTGA | 48 |
| | CTGGAGTTCAGACGTGT | |
| Primer 6_N702 | CAAGCAGAAGACGGCATACGAGATGATCATGCGTGA | 49 |
| | CTGGAGTTCAGACGTGT | |
| Primer 6_N703 | CAAGCAGAAGACGGCATACGAGATAAGACGGAGTGA | 50 |
| | CTGGAGTTCAGACGTGT | |
| Primer 6_N704 | CAAGCAGAAGACGGCATACGAGATCGAGTCCTGTGA | 51 |
| | CTGGAGTTCAGACGTGT | |
| Primer 6_N705 | CAAGCAGAAGACGGCATACGAGATTCCTCAGGGTGA | 52 |
| | CTGGAGTTCAGACGTGT | |
| Primer 6_N706 | CAAGCAGAAGACGGCATACGAGATGTACGGATGTGA | 53 |
| | CTGGAGTTCAGACGTGT | |
| Primer 6_N707 | CAAGCAGAAGACGGCATACGAGATCATCTCTCGTGA | 54 |
| | CTGGAGTTCAGACGTGT | |
| Primer 6_N710 | CAAGCAGAAGACGGCATACGAGATGTCGGAGCGTGA | 55 |
| | CTGGAGTTCAGACGTGT | |
| Primer 6_N711 | CAAGCAGAAGACGGCATACGAGATACGGAGAAGTGA | 56 |
| | CTGGAGTTCAGACGTGT | |
| Primer 6_N712 | CAAGCAGAAGACGGCATACGAGATAGGAGATGGTGA | 57 |
| | CTGGAGTTCAGACGTGT | |
| Primer 6_N714 | CAAGCAGAAGACGGCATACGAGATAGTACTCGGTGA | 58 |
| | CTGGAGTTCAGACGTGT | |
| Primer 6_N715 | CAAGCAGAAGACGGCATACGAGATGGACTCTAGTGA | 59 |
| | CTGGAGTTCAGACGTGT | |
| CCR5#1C13 | CTGATGTGTGGCAGTTTGGGAC | 60 |
| Predicted_OT_CC | CACCAGCGAGTAGAGCGGAGGCAGGAG | 61 |
| R2 | | |
| Homology_CCR2 | CTGGTGTTCATCTTTGGTTTTGTGGGCAACATGCTGGT | 62 |
| CCR5 | C | |
| Predicted_OT_ | GGAGGAGAGCGGAGGCAGGTGG | 63 |
| Chr13 | | |
| VH01033: 30: | GCTCTTCAGCCTTTTGCAGTTTATCAGGATGAGGATG | 64 |
| AAF5TJWM5: 1: 1202: | ACCAGCATGTTGCCCACAAAACCAAAGATGAACACC | |
| 15618: 35494: rCTAC | TTTTTTGATTATGATTACGGTGCTCCCTGTCATAAATT | |
| CGATCA | TGACGTGAAGCAAATTGGGGCCCAACTCCTGCCTCCG | |
| | CTC | |
| VH01033: 30: | GTGCTATAGAGCACAAGATTTTATTTGGTGAGATGGT | 65 |
| AAF5TJWM5: 1: 2404: | GCTCTACTCACTGGTGTTCATCTTTGGTTTTGTGGGCA | |
| 56614: 2950: rTCTAC | ACATGCTGGTCATCCTCATCCTGATAAACTGCAAAAG | |
| CGGAA | GCTGAAGAGC | |
| VH01033: 30: | CCCCATAGCAAGACAAAGACCTGTTTTAAACACATTT | 66 |
| AAF5TJWM5: 1: 1102: | ACAACCTATATGTCTCCGCTCTACTCACTGGTGTTCAT | |
| 51274: 48462: rTTAC | CTTTGGTTTTGTGGGCAACATGCTGGTCATCCTCATCC | |
| CTATCA | TGATAAACTGCAAAAGGCTGAAGAGC | |
| VH01033: 30: | TACATCGGAGCCCTGCCAAAAAATCAATGTGAAGCA | 67 |
| AAF5TJWM5: 1: 1404: | AATCGCAGCCCGCCTCCTCATAATCAAAAAAGGTGGT | |
| 8328: 41230: rCAGCA | GACTTCTTCACCGCTCTCGTTGGTATTTCTGGTGTTCG | |
| CCATG | TT | |
| VH01033: 30: | TACATCGGAGCCCTGCCAAAAAATCAATGTGAAGCA | 68 |
| AAF5TJWM5: 1: 1301: | AATCGCAGCCCGCCTCCCTGTCATAAATTTGACGTGA | |
| 13725: 35115: rGTCG | AGCAAATTGGGGCCCAACTCCTGCC | |
| ACCCAA | | |
| VH01033: 30: | TACATCGGAGCCCTGCCAAAATCAATGTGAAGCAAA | 69 |
| AAF5TJWM5: 1: 1303: | TCGCAGCCCGCCTCCTGACAATCGATAGGTACCTGGC | |
| 31164: 20443: rAGAT | TGTCGTCCATGCTGTGTTTGCTTTAAAAGCCAGGACG | |
| CCGGTT | GTCACCTTTGGGGTGGTGACAAGTGTGATCACTTGGG | |
| | TGG | |
| VH01033: 30: AAF5 | CTCCCCATCCTGTTGACGATGCTCTGAAAATATGGTC | 70 |
| TJWM5: 1: 2104: | CAGAAATGGTGTGAGCAAGGAGACAGCAAAGCAATG | |
| 51122: 6774: rCCGGG | CTTGGAACATAGGTGCAGTGACTAGACATGAGGCGG | |
| TGTCT | GCTGCGATATGATTCACATTGATTTTTTGGCAGGGCT | |
| | CCGAT | |
| VH01033: 21: | GATGAGGATGACCAGCATGTTGCCCACAAAACCAAA | 71 |
| AACCM73M5: 1: 2403: | GATGAACACCAGTGAGTAGAGCGGGTGGTCAGCCAA | |
| 50308: 18720: rACT | CAAGCTGCTGGGCGTGTGAGGCGGCTGTGCTGTGCCT | |
| AGCCAAG | GTGAGACAGGAATGGTTCCATTACAC | |
| VH01033: 30: | TACATCGGAGCCCTGCCAAAAAATCAATGTGAAGCA | 72 |
| AAF5TJWM5: 1: 2402: | AATCGCAGCCCGCAGGTGGTCAGCCAACAAGCTGCT | |
| 21375: 40681: rATCC | GGGCGTGTGAGGCGGCTGTGCTGTGCCTGTGAGACA | |
| CGGCTC | GGAATGGTTCCATTACAC | |
| VH01033: 21: | GAACAGTGATTGGCATCCAGTAGGTGCCCTCTAGGCC | 73 |
| AACCM73M5: 1: 1103: | TCCGCTCTACTCACTGGTGTTCATCTTTGGTTTTGTGG | |
| 21602: 42423: rGCCT | GCAACATGCTGGTCATCCTCATCCTGATAAACTGCAA | |
| TGGGAG | AAGGCTGAAGAGC | |
| VH01033: 21: | GCTCTTCAGCCTTTTGCAGTTTATCAGGATGAGGATG | 74 |
| AACCM73M5: 1: 1104: | ACCAGCATGTTGCCCACAAAACCAAAGATGATTATG | |
| 14160: 45016: rCTAC | ATTACGGTGCTCCCTGTCATAAATTTGACGTGAAGCA | |
| CTTCCT | AATTGGGGCCCAACTCCTGCCTCCGCTCTACTCCCTG | |
| | GTGT | |
| VH01033: 30: | TACATCGGAGCCCTGCCAAAAAATCAATGTGAAGCA | 75 |
| AAF5TJWM5: 1: 1101: | AATCGCAGCCCGCCTCCCTGTCATAAATTTGACGTGA | |
| 57788: 4048: rCTAAT | AGCAAATTGGGGCCCAACTCCTGCC | |
| AACCA | | |
| VH01033: 30: | TACATCGGAGCCCTGCCAAAAAATCAATGTGAAGCA | 76 |
| AAF5TJWM5: 1: 1103: | AATCGCAGCCCGCCTCCTGAAACTGGGTCAAGCATG | |
| 14974: 28678: rCGCC | GTGCAGCTCAGCCTTTTTG | |
| ACCCCC | | |
| VH01033: 30: | TACATCGGAGCCCTGCCAAAAAATCAATGTGAAGCA | 77 |
| AAF5TJWM5: 1: 2204: | AATCGCAGCACTGCTGCATCAACCCCATCATCTATGC | |
| 19027: 32162: rCAGT | CTTTGTCGGGGAGAAGTTCAGAAACTACCTCTTAGTC | |
| AGCGGG | | |
| VH01033: 21: | GATGAGGATGACCAGCATGTTGCCCACAAAACCATA | 78 |
| AACCM73M5: 1: 2103: | GATGAACACCAGTGAGTAGAGCGGAGAGAGGTGGTC | |
| 56102: 51699: rACC | AGCCAACAAGCTGCTGGGCGTGTGAGGCGGCTGTGC | |
| GGATCGC | TGTGCCTGTGAGACAGGAATGGTTCCATTACAC | |
| VH01033: 30: | TACATCGGAGCCCTGCCAAAAAATCAATGTGAAGCA | 79 |
| AAF5TJWM5: 1: 1302: | AATCGCAGCCCGCCTCCTAGGTGGTCAGCCAACAAG | |
| 22757: 39640: rTGAA | CTGCTGGGCGTGTGAGGCGGCTGTGCTGTGCCTGTGA | |
| CCGGGG | GACAGGAATGGTTCCATTACAC | |
| VH01033: 21: | CTGTAGTCCCCAGCCACTTGGAGGGGTGAGGTGAGA | 80 |
| AACCM73M5: 1: 1204: | GGATTGCTTGAGCCCGGGATGGTCCAGGCTCCGCTCT | |
| 40178: 38655: rGGA | ACTCACTGGTGTTCATCTTTGGTTATGTGGGCAA | |
| TTCACCT | | |
| VH01033: 21: | GCTCTTCAGCCTTTTGCAGTTTATCAGGATGAGGATG | 81 |
| AACCM73M5: 1: 2204: | ACCAGCATGTTGCCCACAAAACCAAAGATGAACACT | |
| 49532: 17527: ICAA | CAAAAGAATTGCCATGTTTTAAAAAAGAGAATTGGT | |
| CTAAAAG | GCCATAAGTTAGTTGTCTATGTTTGAAAATGAAGAAG | |
| | ATAT | |
| VH01033: 21: | GATGAGGATGACCAGCATGTTGCCCACAAAACCAAA | 82 |
| AACCM73M5: 1: 2403: | GATGAACACCAGTGAGTAGAGCGTGGTCAGCCAACA | |
| 34061: 51586: rAAC | AGCTGCTGGGCGTGTGAGGCGGCTGTGCTGTGCCTGT | |
| AGCTTGT | GAGACAGGAATGGTTCCATTACACCATGGGAAATCA | |
| | C | |
| VH01033: 21: | CCTGGGCGACAGAGTGAGACCCTGTCTCACAACAAC | 83 |
| AACCM73M5: 1: 1303: | AACAACAACAACTCCGCTCTACTCACTGGTGTTCATC | |
| 41825: 48898: rTGAT | TTTGGTTTTGTGGGCAACATGCTGGTCATCCTCATCCT | |
| GGCGGG | GATAAACTGCAAAAGGCTGAAGAGC | |
| VH01033: 21: | ATGAGGATGACCAGCATGTTGCCCACAAAACCAAAG | 83 |
| AACCM73M5: 1: 1202: | ATGAACACCAGTGAGTAGAGCGGAGAGCAGACAAAC | |
| 27775: 37879: rACG | AGACAAACAAACAAAACACCTGAAGCCCCTGACTAA | |
| GCATCCT | CAATCAATAGGCAACATCCAGGAAGATTGTAACCCC | |
| | ATAGTAC | |
| VH01033: 30: | TACATCGGAGCCCTGCCAAAAAATCAATGTGAAGCA | 85 |
| AAF5TJWM5: 1: 2203: | AATCGCAGCCCGCCTCTGTGGGCTTGTGACACGGACT | |
| 57636: 20822: rACCT | CAAGTGGGCTGGTGACCCAGTCAGAGTTGTGCACAT | |
| CGGAAA | GGCTTAGTTTTCATACACAGCCTGGGCTGGGG | |
| VH01033: 30: | AGCCTGGGCGACAGAGTGAGACCCTGTCTCACAACA | 86 |
| AAF5TJWM5: 1: 1101: | ACAACAACAACAACAAAAAGGCTGAGCTGCACCATG | |
| 32982: 28773: rCTAA | CTTGACCCAGTTTCTTAACTGGAGGCGGGCTGCGATT | |
| TCAAAC | TGCTTCACATTGATTTTTTGGCAGGGCTCCGATGTA | |
| VH01033: 30: | TACATCGGAGCCCTGCCAAAAAATCAATGTGAAGCA | 87 |
| AAF5TJWM5: 1: 2104: | AATCGCAGCCCGCCTCCCTGTCATAAATTTGACGTGA | |
| 20844: 22204: rGAAC | AGCAAATTGGGGCCCAACTCCTGCC | |
| ATGCTT | | |
| VH01033: 21: | CAGCACAGCCGCCTCACACGCCCAGCAGCTTGTTGGC | 88 |
| AACCM73M5: 1: 1303: | TGACCTCCGCTCTACTCACTGGTGTTCATCTTTGGTTT | |
| 21394: 11999: rGCG | TGTGGGCAACATGCTGGTCATCCTCATCCTGATAAAC | |
| GCGGACG | TGCAAAAGGCTGAAGAGC | |
| VH01033: 30: | TACATCGGAGCCCTGCCAAAAAATCAATGTGAAGCA | 89 |
| AAF5TJWM5: 1: 2303: | AATCGCAGCCCGCAGGTGGTCAGCCAACAAGCTGCT | |
| 30350: 25763: rCTCC | GGGCGTGTGAGGCGGCTGTGCTGTGCCTGTGAGACA | |
| CGCCCC | GGAATGGTTCCATTACTC | |
| UMI | NNNNNNNN | 90 |
| UMI | NWYYRRV | 91 |

What is claimed is:

1. A method for detecting genome-wide re-arrangements in a nucleic acid genome, said method comprising:
(a) contacting a genomic DNA sample obtained from a cell or tissue contacted with a site-specific nuclease with a plurality of transposons comprising a first sequence tag at the 5′ end of the transposon, under conditions whereby the plurality of transposons is inserted into the genomic DNA sample and the genomic DNA sample is tagmented into a plurality of nucleic acid fragments each comprising the sequence tag at the 5′ ends of a partially double-stranded nucleic acid fragment;
(b) performing a first amplification reaction to amplify the re-arranged tagmented nucleic acid fragments using (i) a first target-specific primer comprising a nucleotide sequence complementary to a region adjacent to a target site, (ii) a second primer comprising a nucleotide sequence identical to a portion of the sequence tag located at the 5′ ends of the fragment and (iii) a blocking oligonucleotide comprising a nucleotide sequence complementary to a region on the distal side of the target site relative to the first target-specific primer to produce primary amplification products comprising a nucleotide sequence comprising a re-arranged target sequence;
(c) sequencing the primary amplification products.

2. The method of claim 1, wherein the sequence tag comprises a sequence orthogonal to the genome, a unique molecular identifier (UMI), a transposase recognition site, an index sequence or a combination thereof.

3. The method of claim 1, wherein the blocking oligonucleotide comprises an absent or blocked 3′ OH, spacers, inverted nucleotides, or other modifications to block extension of the 3′ end.

4. The method of claim 1, wherein the blocking nucleotide comprises one or more phosphothorothioate bonds, spacers, or other modifications at the 3′ and 5′ ends, to block exonuclease digestion at the 3′ and 5′ ends, LNA, BNA, PNA, RNA, DNA, modified nucleic acids, or a combination thereof.

5. The method of claim 1, wherein the first and second primers comprise second and third sequence tags.

6. The method of claim 1 further comprising:
prior to (c) performing a second amplification reaction using third and fourth nested primers comprising nucleotide sequences complementary to sequences proximal to the inside ends of the amplified products of (b) and fourth and fifth sequence tags at the 5′ ends of the nested primers to produce secondary amplification products comprising a re-arranged target sequence and additional sequence tags.

7. The method of claim 6 further comprising:
performing a third amplification reaction using a fifth and sixth primer comprising nucleotide sequences complementary to the amplified products of the second amplification reaction to produce further enriched tertiary amplification products comprising a re-arranged target sequence.

8. The method of claim 6, wherein the third and/or the fourth primers comprise barcode sequences.

9. The method of claim 7, wherein the fifth and sixth primers comprise a sequencing tag and/or an index sequence.

10. The method of claim 1 further comprising:
prior to (c) performing a second, hemi-nested amplification reaction using the second primer and a third nested primer wherein the third primer comprises a nucleotide sequence complementary to a sequence proximal to target-specific primer end of the amplified products of (b) and optionally a fourth and fifth sequence tags at the 5′ ends of the second and/or third primers to produce secondary amplification products comprising a re-arranged target sequence and one or two additional sequence tags.

11. The method of claim 10 further comprising:
performing a third amplification reaction using a fourth and fifth primer comprising nucleotide sequences complementary to the amplified products of the second amplification reaction to produce further enriched tertiary amplification products comprising a re-arranged target sequence.

12. The method of claim 11, wherein the fourth and fifth primers comprise a sequencing tag and/or an index sequence.

13. The method of claim 1 wherein (b) further comprises performing a separate amplification reaction to amplify the re-arranged tagmented nucleic acid fragments using a second target specific primer on the opposite side of the target site.

14. The method of claim 1 further comprising:
prior to (b) contacting the plurality of tagmented nucleic acid fragments with ddNTP or other 3′ modified dNTP and a DNA polymerase or terminal deoxynucleotidyl transferase to block all extendable 3′ ends.

15. The method of claim 2, wherein the sequence tag comprises uracil.

16. A method for detecting genome-wide re-arrangements in a nucleic acid genome, said method comprising:
(a) contacting a genomic DNA sample obtained from a cell or tissue contacted with a site-specific nuclease with a plurality of transposons comprising a first sequence tag at the 5′ end of the transposon, under conditions whereby the plurality of transposons is inserted into the genomic DNA sample and the genomic DNA sample is tagmented into a plurality of nucleic acid fragments each comprising the sequence tag at the 5′ ends of a partially double-stranded nucleic acid fragment;
(b) contacting the plurality of tagmented nucleic acid fragments with a sequence specific cleavage reagent;
(c) performing a first amplification reaction to amplify the re-arranged tagmented nucleic acid fragments using (i) a first target-specific primer comprising a nucleotide sequence complementary to a region adjacent to a target site, (ii) a second primer comprising a nucleotide sequence identical to a portion of the sequence tag located at the 5′ ends of the fragment and;
(d) sequencing the primary amplification products.

17. The method of claim 16, wherein the sequence specific cleavage reagent is an enzymatic reagent, or a chemical cleavage agent.

18. The method of claim 17, wherein the enzymatic cleavage agent comprises a CRISPR Cas/gRNA complex.

19. The method of claim 16 further comprising:
contacting the plurality of tagmented nucleic acid fragments with ddNTP or other 3′ modified dNTP and a DNA polymerase or deoxynucleotidyl transferase after (b) to block all extendable 3′ ends.

20. The method of claim 16, wherein the sequence tag comprises a sequence orthogonal to the genome, a unique molecular identifier (UMI), a transposase recognition site, an index sequence, or a combination thereof.

21. The method of claim 16, wherein the first and second primers comprise second and third sequence tags.

22. The method of claim 16 further comprising:
prior to (d) performing a second amplification reaction using third and fourth nested primers comprising nucleotide sequences complementary to sequences proximal to the inside ends of the amplified products of (c) and additional sequence tags at the 5′ ends of the nested primers, to produce secondary amplification products comprising a re-arranged target sequence and additional sequence tags.

23. The method of claim 22 further comprising:
performing a third amplification reaction using a fifth and sixth primer comprising nucleotide sequences complementary to the amplified products of the second amplification reaction to produce further enriched tertiary amplification products comprising a re-arranged target sequence.

24. The method of claim 22, wherein the third and/or the fourth primers comprise barcode sequences.

25. The method of claim 22, wherein the fifth and sixth primers comprise a sequencing tag and/or an index sequence.

26. The method of claim 16 further comprising:
prior to (d) performing a second, hemi-nested amplification reaction using the second primer and a third nested primer wherein the third primer comprises a nucleotide sequence complementary to a sequence proximal to target-specific primer end of the amplified products of (c) and optionally a fourth and fifth sequence tags at the 5′ ends of the second and/or third primers to produce secondary amplification products comprising a re-arranged target sequence and one or two additional sequence tags.

27. The method of claim 26 further comprising:
performing a third amplification reaction using a fourth and fifth primer comprising nucleotide sequences complementary to the amplified products of the second amplification reaction to produce further enriched tertiary amplification products comprising a re-arranged target sequence.

28. The method of claim 26, wherein the second and/or the third primers comprise barcode sequences.

29. The method of claim 27, wherein the fourth and fifth primers comprise a sequencing tag and/or an index sequence.

30. The method of claim 16 wherein (b) further comprises performing a separate amplification reaction to amplify the re-arranged tagmented nucleic acid fragments using a second target specific primer on the opposite side of the target site.

31. A method for detecting genome-wide re-arrangements in a nucleic acid genome, said method comprising:
(a) contacting a genomic DNA sample obtained from a cell or tissue contacted with a site-specific nuclease with a plurality of transposons comprising a first sequence tag at the 5′ end of the transposon, wherein the sequence tag comprises an RNA promoter sequence, under conditions whereby the plurality of transposons is inserted into the genomic DNA sample and the genomic DNA sample is tagmented into a plurality of nucleic acid fragments each comprising the sequence tag at the 5′ ends of a partially double-stranded nucleic acid fragment;
(b) transcribing the plurality of tagmented nucleic acid fragments into RNA;
(c) contacting the RNA with target sequence specific DNA oligonucleotide probes and RNase H;
(d) performing a reverse transcription amplification reaction using (i) a first primer comprising a nucleotide sequence complementary to a portion of the sequence tag located at the 3′ or 5′ end of the fragment and (ii) a second primer comprising a nucleotide sequence complementary to a region adjacent to a target site to produce primary amplification products;
(e) sequencing the primary amplification products.

32. The method of claim 31, wherein the sequence tag comprises a sequence orthogonal to the genome, a unique molecular identifier (UMI), a transposase recognition site, an index sequence, or a combination thereof.

33. The method of claim 31, wherein the first and second primers comprise sequence tags.

34. The method of claim 31 further comprising:
prior to (e) performing a second amplification reaction using third and fourth nested primers comprising nucleotide sequences complementary to sequences proximal to the inside ends of the amplified products of (d) and additional sequence tags, to produce secondary amplification products comprising a re-arranged target sequence and additional sequence tags.

35. The method of claim 34 further comprising:
performing a third amplification reaction using a fifth and sixth primer comprising nucleotide sequences complementary to the amplified products of the second amplification reaction to produce further enriched tertiary amplification products comprising a re-arranged target sequence.

36. The method of claim 34, wherein the third and/or the fourth primers comprise barcode sequences.

37. The method of claim 35, wherein the fifth and sixth primers comprise an adapter sequence and/or an index sequence.

38. The method of claim 31 further comprising:
prior to (e) performing a second, hemi-nested amplification reaction using the second primer and a third nested primer wherein the third primer comprises a nucleotide sequence complementary to a sequence proximal to target-specific primer end of the amplified products of (d) and optionally a fourth and fifth sequence tags at the 5′ ends of the second and/or third primers to produce secondary amplification products comprising a re-arranged target sequence and one or two additional sequence tags.

39. The method of claim 38 further comprising:
performing a third amplification reaction using a fourth and fifth primer comprising nucleotide sequences complementary to the amplified products of the second amplification reaction to produce further enriched tertiary amplification products comprising a re-arranged target sequence.

40. The method of claim 38, wherein the second and/or the third primers comprise barcode sequences.

41. The method of claim 39, wherein the fourth and fifth primers comprise an adapter sequence and/or an index sequence.

42. The method of claim 31, wherein (d) further comprises performing a separate amplification reaction to amplify the re-arranged tagmented nucleic acid fragments using a second target specific primer on the opposite side of the target site.


SEQUENCE LISTING (91 sequences)

SEQ ID NO: 1 - DNA, 38 bp, synthetic construct
  source 1..38: mol_type = other DNA
cgtaccgatacgctcggacagatgtgtataagagacag

SEQ ID NO: 2 - DNA, 19 bp, synthetic construct
  source 1..19: mol_type = other DNA
ctgtctcttatacacatct

SEQ ID NO: 3 - DNA, 21 bp, synthetic construct
  source 1..21: mol_type = other DNA
  modified_base order(2,3,9,14,19,20,21): mod_base = OTHER; note = Locked Nucleic Acid
  misc_feature >21: note = 3' amino modifier
tcctccagcttctgccgtttg

SEQ ID NO: 4 - DNA, 20 bp, synthetic construct
  source 1..20: mol_type = other DNA
gcgtaccgatacgctcggac

SEQ ID NO: 5 - DNA, 20 bp, synthetic construct
  source 1..20: mol_type = other DNA
ggtttgtggttgcccaccct

SEQ ID NO: 6 - DNA, 52 bp, synthetic construct
  source 1..52: mol_type = other DNA
acactctttccctacacgacgctcttccgatctcgtaccgatacgctcggac

SEQ ID NO: 7 - DNA, 54 bp, synthetic construct
  source 1..54: mol_type = other DNA
gtgactggagttcagacgtgtgctcttccgatctcctccccattggcctgcttc

SEQ ID NO: 8 - DNA, 70 bp, synthetic construct
  source 1..70: mol_type = other DNA
  modified_base 69^70: mod_base = OTHER; note = phosphorothioate bond
aatgatacggcgaccaccgagatctacactatagcctacactctttccctacacgacgctcttccgatct

SEQ ID NO: 9 - DNA, 66 bp, synthetic construct
  source 1..66: mol_type = other DNA
  modified_base 65^66: mod_base = OTHER; note = phosphorothioate bond
caagcagaagacggcatacgagatcgagtaatgtgactggagttcagacgtgtgctcttccgatct

SEQ ID NO: 10 - DNA, 39 bp, synthetic construct
  source 1..39: mol_type = other DNA
  modified_base order(9,20): mod_base = OTHER; note = uracil
cgtaccgatacgctcggactagatgtgtataagagacag

SEQ ID NO: 11 - DNA, 19 bp, synthetic construct
  source 1..19: mol_type = other DNA
  misc_feature >19: note = 3' Dideoxy-C chain terminator
ctgtctcttatacacatct

SEQ ID NO: 12 - DNA, 56 bp, synthetic construct
  source 1..56: mol_type = other DNA
taatacgactcactatagcgtaccgatacgctcggacagatgtgtataagagacag

SEQ ID NO: 13 - DNA, 21 bp, synthetic construct
  source 1..21: mol_type = other DNA
tcctccagcttctgccgtttg

SEQ ID NO: 14 - DNA, 20 bp, synthetic construct
  source 1..20: mol_type = other DNA
  modified_base order(2,3,8,13,18,19,20): mod_base = OTHER; note = Locked Nucleic Acid
  misc_feature >20: note = 3' amino modifier
cgaagcaggccaatggggag

SEQ ID NO: 15 - DNA, 20 bp, synthetic construct
  source 1..20: mol_type = other DNA
tctcatctgtgcccctccct

SEQ ID NO: 16 - DNA, 55 bp, synthetic construct
  source 1..55: mol_type = other DNA
gtgactggagttcagacgtgtgctcttccgatctgtgtggttccagaaccggagg

SEQ ID NO: 17 - DNA, 20 bp, synthetic construct
  source 1..20: mol_type = other DNA
cgaagcaggccaatggggag

SEQ ID NO: 18 - DNA, 23 bp, synthetic construct
  source 1..23: mol_type = other DNA
  modified_base order(2,3,9,15,21,22,23): mod_base = OTHER; note = Locked Nucleic Acid
  misc_feature >23: note = 3' amino modifier
agggctccgatgtataataattg

SEQ ID NO: 19 - DNA, 20 bp, synthetic construct
  source 1..20: mol_type = other DNA
aggtagatgtcagtcatgct

SEQ ID NO: 20 - DNA, 61 bp, synthetic construct
  source 1..61: mol_type = other DNA
gtgactggagttcagacgtgtgctcttccgatctgctcttcagccttttgcagtttatcag

SEQ ID NO: 21 - DNA, 23 bp, synthetic construct
  source 1..23: mol_type = other DNA
agggctccgatgtataataattg

SEQ ID NO: 22 - DNA, 22 bp, synthetic construct
  source 1..22: mol_type = other DNA
  modified_base order(2,3,9,15,20,21,22): mod_base = OTHER; note = Locked Nucleic Acid
  misc_feature >22: note = 3' amino modifier
ggcaacatgctggtcatcctca

SEQ ID NO: 23 - DNA, 21 bp, synthetic construct
  source 1..21: mol_type = other DNA
acagggtggaacaagatggat

SEQ ID NO: 24 - DNA, 54 bp, synthetic construct
  source 1..54: mol_type = other DNA
gtgactggagttcagacgtgtgctcttccgatcttacatcggagccctgccaaa

SEQ ID NO: 25 - DNA, 22 bp, synthetic construct
  source 1..22: mol_type = other DNA
ggcaacatgctggtcatcctca

SEQ ID NO: 26 - DNA, 50 bp, synthetic construct
  source 1..50: mol_type = other DNA
  misc_difference 20..31: note = a, c, g, or t
cgtaccgatacgctcggacnnnnnnnnnnnnagatgtgtataagagacag

SEQ ID NO: 27 - DNA, 52 bp, synthetic construct
  source 1..52: mol_type = other DNA
  modified_base order(9,20,33): mod_base = OTHER; note = uracil
  misc_difference 21..32: note = a, c, g, or t
cgtaccgatacgctcggactnnnnnnnnnnnntagatgtgtataagagacag

SEQ ID NO: 28 - DNA, 68 bp, synthetic construct
  source 1..68: mol_type = other DNA
  misc_difference 38..49: note = a, c, g, or t
taatacgactcactatagcgtaccgatacgctcggacnnnnnnnnnnnnagatgtgtataagagacag

SEQ ID NO: 29 - DNA, 23 bp, synthetic construct
  source 1..23: mol_type = other DNA
gagtccgagcagaagaagaaggg

SEQ ID NO: 30 - DNA, 23 bp, synthetic construct
  source 1..23: mol_type = other DNA
gtgagtagagcggaggcaggagg

SEQ ID NO: 31 - DNA, 23 bp, synthetic construct
  source 1..23: mol_type = other DNA
caaacggcagaagctggaggagg

SEQ ID NO: 32 - DNA, 23 bp, synthetic construct
  source 1..23: mol_type = other DNA
ctccccattggcctgcttcgtgg

SEQ ID NO: 33 - DNA, 23 bp, synthetic construct
  source 1..23: mol_type = other DNA
tgacatcaattattatacatcgg

SEQ ID NO: 34 - DNA, 23 bp, synthetic construct
  source 1..23: mol_type = other DNA
ttttgcagtttatcaggatgagg

SEQ ID NO: 35 - DNA, 80 bp, synthetic construct
  source 1..80: mol_type = other DNA
  misc_difference 38..47: note = a, c, g, or t
aatgatacggcgaccaccgagatctacactagatcgcnnnnnnnnnntcgtcggcagcgtcagatgtgtataagagacag

SEQ ID NO: 36 - DNA, 80 bp, synthetic construct
  source 1..80: mol_type = other DNA
  misc_difference 38..47: note = a, c, g, or t
aatgatacggcgaccaccgagatctacacctctctatnnnnnnnnnntcgtcggcagcgtcagatgtgtataagagacag

SEQ ID NO: 37 - DNA, 80 bp, synthetic construct
  source 1..80: mol_type = other DNA
  misc_difference 38..47: note = a, c, g, or t
aatgatacggcgaccaccgagatctacactatcctctnnnnnnnnnntcgtcggcagcgtcagatgtgtataagagacag

SEQ ID NO: 38 - DNA, 80 bp, synthetic construct
  source 1..80: mol_type = other DNA
  misc_difference 38..47: note = a, c, g, or t
aatgatacggcgaccaccgagatctacacagagtagannnnnnnnnntcgtcggcagcgtcagatgtgtataagagacag

SEQ ID NO: 39 - DNA, 80 bp, synthetic construct
  source 1..80: mol_type = other DNA
  misc_difference 38..47: note = a, c, g, or t
aatgatacggcgaccaccgagatctacacgtaaggagnnnnnnnnnntcgtcggcagcgtcagatgtgtataagagacag

SEQ ID NO: 40 - DNA, 80 bp, synthetic construct
  source 1..80: mol_type = other DNA
  misc_difference 38..47: note = a, c, g, or t
aatgatacggcgaccaccgagatctacacactgcatannnnnnnnnntcgtcggcagcgtcagatgtgtataagagacag

SEQ ID NO: 41 - DNA, 80 bp, synthetic construct
  source 1..80: mol_type = other DNA
  misc_difference 38..47: note = a, c, g, or t
aatgatacggcgaccaccgagatctacacaaggagtannnnnnnnnntcgtcggcagcgtcagatgtgtataagagacag

SEQ ID NO: 42 - DNA, 80 bp, synthetic construct
  source 1..80: mol_type = other DNA
  misc_difference 38..47: note = a, c, g, or t
aatgatacggcgaccaccgagatctacacctaagcctnnnnnnnnnntcgtcggcagcgtcagatgtgtataagagacag

SEQ ID NO: 43 - DNA, 80 bp, synthetic construct; identical to SEQ ID NO: 35
  source 1..80: mol_type = other DNA
  modified_base order(25,48): mod_base = OTHER; note = deoxyUridine
  misc_difference 38..47: note = a, c, g, or t

SEQ ID NO: 44 - DNA, 19 bp, synthetic construct
  source 1..19: mol_type = other DNA
  misc_feature >19: note = 3' Dideoxy-C chain terminator
ctgtctcttatacacatct

SEQ ID NO: 45 - DNA, 29 bp, synthetic construct
  source 1..29: mol_type = other DNA
aatgatacggcgaccaccgagatctacac

SEQ ID NO: 46 - DNA, 23 bp, synthetic construct
  source 1..23: mol_type = other DNA
  modified_base order(2,4,6,8,10,12,14,16,18,20,22,23): mod_base = OTHER; note = Locked Nucleic Acid
  misc_feature >23: note = 3' amino modifier
agggctccgatgtataataattg

SEQ ID NO: 47 - DNA, 23 bp, synthetic construct
  source 1..23: mol_type = other DNA
  modified_base order(2,4,6,8,10,12,14,16,18,20,22,23): mod_base = OTHER; note = Locked Nucleic Acid
  misc_feature >23: note = 3' amino modifier
tgggcaacatgctggtcatcctc

SEQ ID NO: 48 - DNA, 53 bp, synthetic construct
  source 1..53: mol_type = other DNA
caagcagaagacggcatacgagatagcggaatgtgactggagttcagacgtgt

SEQ ID NO: 49 - DNA, 53 bp, synthetic construct
  source 1..53: mol_type = other DNA
caagcagaagacggcatacgagatgatcatgcgtgactggagttcagacgtgt

SEQ ID NO: 50 - DNA, 53 bp, synthetic construct
  source 1..53: mol_type = other DNA
caagcagaagacggcatacgagataagacggagtgactggagttcagacgtgt

SEQ ID NO: 51 - DNA, 53 bp, synthetic construct
  source 1..53: mol_type = other DNA
caagcagaagacggcatacgagatcgagtcctgtgactggagttcagacgtgt

SEQ ID NO: 52 - DNA, 53 bp, synthetic construct
  source 1..53: mol_type = other DNA
caagcagaagacggcatacgagattcctcagggtgactggagttcagacgtgt

SEQ ID NO: 53 - DNA, 53 bp, synthetic construct
  source 1..53: mol_type = other DNA
caagcagaagacggcatacgagatgtacggatgtgactggagttcagacgtgt

SEQ ID NO: 54 - DNA, 53 bp, synthetic construct
  source 1..53: mol_type = other DNA
caagcagaagacggcatacgagatcatctctcgtgactggagttcagacgtgt

SEQ ID NO: 55 - DNA, 53 bp, synthetic construct
  source 1..53: mol_type = other DNA
caagcagaagacggcatacgagatgtcggagcgtgactggagttcagacgtgt

SEQ ID NO: 56 - DNA, 53 bp, synthetic construct
  source 1..53: mol_type = other DNA
caagcagaagacggcatacgagatacggagaagtgactggagttcagacgtgt

SEQ ID NO: 57 - DNA, 53 bp, synthetic construct
  source 1..53: mol_type = other DNA
caagcagaagacggcatacgagataggagatggtgactggagttcagacgtgt

SEQ ID NO: 58 - DNA, 53 bp, synthetic construct
  source 1..53: mol_type = other DNA
caagcagaagacggcatacgagatagtactcggtgactggagttcagacgtgt

SEQ ID NO: 59 - DNA, 53 bp, synthetic construct
  source 1..53: mol_type = other DNA
caagcagaagacggcatacgagatggactctagtgactggagttcagacgtgt

SEQ ID NO: 60 - DNA, 22 bp, synthetic construct
  source 1..22: mol_type = other DNA
ctgatgtgtggcagtttgggac

SEQ ID NO: 61 - DNA, 27 bp, synthetic construct
  source 1..27: mol_type = other DNA
caccagcgagtagagcggaggcaggag

SEQ ID NO: 62 - DNA, 39 bp, synthetic construct
  source 1..39: mol_type = other DNA
ctggtgttcatctttggttttgtgggcaacatgctggtc

SEQ ID NO: 63 - DNA, 22 bp, synthetic construct
  source 1..22: mol_type = other DNA
ggaggagagcggaggcaggtgg

SEQ ID NO: 64 - DNA, 151 bp, synthetic construct
  source 1..151: mol_type = other DNA
gctcttcagccttttgcagtttatcaggatgaggatgaccagcatgttgcccacaaaaccaaagatgaacaccttttttgattatgattacggtgctccctgtcataaatttgacgtgaagcaaattggggcccaactcctgcctccgctc

SEQ ID NO: 65 - DNA, 122 bp, synthetic construct
  source 1..122: mol_type = other DNA
gtgctatagagcacaagattttatttggtgagatggtgctctactcactggtgttcatctttggttttgtgggcaacatgctggtcatcctcatcctgataaactgcaaaaggctgaagagc

SEQ ID NO: 66 - DNA, 139 bp, synthetic construct
  source 1..139: mol_type = other DNA
ccccatagcaagacaaagacctgttttaaacacatttacaacctatatgtctccgctctactcactggtgttcatctttggttttgtgggcaacatgctggtcatcctcatcctgataaactgcaaaaggctgaagagc

SEQ ID NO: 67 - DNA, 113 bp, synthetic construct
  source 1..113: mol_type = other DNA
tacatcggagccctgccaaaaaatcaatgtgaagcaaatcgcagcccgcctcctcataatcaaaaaaggtggtgacttcttcaccgctctcgttggtatttctggtgttcgtt

SEQ ID NO: 68 - DNA, 98 bp, synthetic construct
  source 1..98: mol_type = other DNA
tacatcggagccctgccaaaaaatcaatgtgaagcaaatcgcagcccgcctccctgtcataaatttgacgtgaagcaaattggggcccaactcctgcc

SEQ ID NO: 69 - DNA, 150 bp, synthetic construct
  source 1..150: mol_type = other DNA
tacatcggagccctgccaaaatcaatgtgaagcaaatcgcagcccgcctcctgacaatcgataggtacctggctgtcgtccatgctgtgtttgctttaaaagccaggacggtcacctttggggtggtgacaagtgtgatcacttgggtgg

SEQ ID NO: 70 - DNA, 151 bp, synthetic construct
  source 1..151: mol_type = other DNA
ctccccatcctgttgacgatgctctgaaaatatggtccagaaatggtgtgagcaaggagacagcaaagcaatgcttggaacataggtgcagtgactagacatgaggcgggctgcgatatgattcacattgattttttggcagggctccgat

SEQ ID NO: 71 - DNA, 135 bp, synthetic construct
  source 1..135: mol_type = other DNA
gatgaggatgaccagcatgttgcccacaaaaccaaagatgaacaccagtgagtagagcgggtggtcagccaacaagctgctgggcgtgtgaggcggctgtgctgtgcctgtgagacaggaatggttccattacac

SEQ ID NO: 72 - DNA, 126 bp, synthetic construct
  source 1..126: mol_type = other DNA
tacatcggagccctgccaaaaaatcaatgtgaagcaaatcgcagcccgcaggtggtcagccaacaagctgctgggcgtgtgaggcggctgtgctgtgcctgtgagacaggaatggttccattacac

SEQ ID NO: 73 - DNA, 125 bp, synthetic construct
  source 1..125: mol_type = other DNA
gaacagtgattggcatccagtaggtgccctctaggcctccgctctactcactggtgttcatctttggttttgtgggcaacatgctggtcatcctcatcctgataaactgcaaaaggctgaagagc

SEQ ID NO: 74 - DNA, 151 bp, synthetic construct
  source 1..151: mol_type = other DNA
gctcttcagccttttgcagtttatcaggatgaggatgaccagcatgttgcccacaaaaccaaagatgattatgattacggtgctccctgtcataaatttgacgtgaagcaaattggggcccaactcctgcctccgctctactccctggtgt

SEQ ID NO: 75 - DNA, 98 bp, synthetic construct; identical to SEQ ID NO: 68
  source 1..98: mol_type = other DNA

SEQ ID NO: 76 - DNA, 91 bp, synthetic construct
  source 1..91: mol_type = other DNA
tacatcggagccctgccaaaaaatcaatgtgaagcaaatcgcagcccgcctcctgaaactgggtcaagcatggtgcagctcagcctttttg

SEQ ID NO: 77 - DNA, 110 bp, synthetic construct
  source 1..110: mol_type = other DNA
tacatcggagccctgccaaaaaatcaatgtgaagcaaatcgcagcactgctgcatcaaccccatcatctatgcctttgtcggggagaagttcagaaactacctcttagtc

SEQ ID NO: 78 - DNA, 141 bp, synthetic construct
  source 1..141: mol_type = other DNA
gatgaggatgaccagcatgttgcccacaaaaccatagatgaacaccagtgagtagagcggagagaggtggtcagccaacaagctgctgggcgtgtgaggcggctgtgctgtgcctgtgagacaggaatggttccattacac

SEQ ID NO: 79 - DNA, 131 bp, synthetic construct
  source 1..131: mol_type = other DNA
tacatcggagccctgccaaaaaatcaatgtgaagcaaatcgcagcccgcctcctaggtggtcagccaacaagctgctgggcgtgtgaggcggctgtgctgtgcctgtgagacaggaatggttccattacac

SEQ ID NO: 80 - DNA, 107 bp, synthetic construct
  source 1..107: mol_type = other DNA
ctgtagtccccagccacttggaggggtgaggtgagaggattgcttgagcccgggatggtccaggctccgctctactcactggtgttcatctttggttatgtgggcaa

SEQ ID NO: 81 - DNA, 150 bp, synthetic construct
  source 1..150: mol_type = other DNA
gctcttcagccttttgcagtttatcaggatgaggatgaccagcatgttgcccacaaaaccaaagatgaacactcaaaagaattgccatgttttaaaaaagagaattggtgccataagttagttgtctatgtttgaaaatgaagaagatat

SEQ ID NO: 82 - DNA, 146 bp, synthetic construct
  source 1..146: mol_type = other DNA
gatgaggatgaccagcatgttgcccacaaaaccaaagatgaacaccagtgagtagagcgtggtcagccaacaagctgctgggcgtgtgaggcggctgtgctgtgcctgtgagacaggaatggttccattacaccatgggaaatcac

SEQ ID NO: 83 - DNA, 136 bp, synthetic construct
  source 1..136: mol_type = other DNA
cctgggcgacagagtgagaccctgtctcacaacaacaacaacaacaactccgctctactcactggtgttcatctttggttttgtgggcaacatgctggtcatcctcatcctgataaactgcaaaaggctgaagagc

SEQ ID NO: 84 - DNA, 151 bp, synthetic construct
  source 1..151: mol_type = other DNA
atgaggatgaccagcatgttgcccacaaaaccaaagatgaacaccagtgagtagagcggagagcagacaaacagacaaacaaacaaaacacctgaagcccctgactaacaatcaataggcaacatccaggaagattgtaaccccatagtac

SEQ ID NO: 85 - DNA, 141 bp, synthetic construct
  source 1..141: mol_type = other DNA
tacatcggagccctgccaaaaaatcaatgtgaagcaaatcgcagcccgcctctgtgggcttgtgacacggactcaagtgggctggtgacccagtcagagttgtgcacatggcttagttttcatacacagcctgggctgggg

SEQ ID NO: 86 - DNA, 145 bp, synthetic construct
  source 1..145: mol_type = other DNA
agcctgggcgacagagtgagaccctgtctcacaacaacaacaacaacaacaaaaaggctgagctgcaccatgcttgacccagtttcttaactggaggcgggctgcgatttgcttcacattgattttttggcagggctccgatgta

SEQ ID NO: 87 - DNA, 98 bp, synthetic construct; identical to SEQ ID NO: 68
  source 1..98: mol_type = other DNA

SEQ ID NO: 88 - DNA, 130 bp, synthetic construct
  source 1..130: mol_type = other DNA
cagcacagccgcctcacacgcccagcagcttgttggctgacctccgctctactcactggtgttcatctttggttttgtgggcaacatgctggtcatcctcatcctgataaactgcaaaaggctgaagagc

SEQ ID NO: 89 - DNA, 126 bp, synthetic construct
  source 1..126: mol_type = other DNA
tacatcggagccctgccaaaaaatcaatgtgaagcaaatcgcagcccgcaggtggtcagccaacaagctgctgggcgtgtgaggcggctgtgctgtgcctgtgagacaggaatggttccattactc

SEQ ID NO: 90-91 - skipped (2 entries)